(19) United States
(12) Patent Application Publication
(10) Pub. No.: US 2026/0367071 A1
(43) Pub. Date: Nov. 19, 2026

(54) COMPOSITIONS AND METHODS FOR EVALUATING MICROTUBULE DYNAMICS

(71) Applicant: The Regents of the University of Michigan, Ann Arbor, MI (US)

(72) Inventors: Takashi Hotta, Ann Arbor, MI (US); Ryoma Ohi, Ann Arbor, MI (US)

(21) Appl. No.: 19/100,186

(22) PCT Filed: Aug. 1, 2023
(86) PCT No.: PCT/US2023/071417

§ 371 Date: Jan. 31, 2025

Related U.S. Application Data
(60) Provisional application No. 63/394,201, filed on Aug. 1, 2022.

(51) Int. Cl. G01N 33/50; C07K 14/47; G01N 33/58

(52) U.S. Cl. CPC G01N 33/5026; C07K 14/4702; G01N 33/582; G01N 2333/4703

(57) ABSTRACT

The present disclosure relates to labeling of proteins for evaluating changes in microtubule structure. Included are compositions and methods that permit detection of shortening and/or lengthening of microtubules for research, diagnostic, and therapeutic purposes.

The present application claims priority to U.S. Provisional Patent Application Ser. No. 63/394,201, filed Aug. 1, 2022, the disclosure of which is herein incorporated by reference in its entirety.

### SEQUENCE LISTING

The text of the computer readable sequence listing filed herewith, titled “40680-601_SEQUENCE_LISTING”, created Aug. 1, 2023, having a file size of 124,467 bytes, is hereby incorporated by reference in its entirety.

### FIELD

The present disclosure relates to labeling of proteins for evaluating changes in microtubule dynamics. Included are compositions and methods that permit detection of shortening and/or lengthening of microtubules for research, diagnostic, and therapeutic purposes.

### BACKGROUND

Microtubules are dynamic polymers assembled from the protein tubulin. Microtubules exhibit dynamic instability, a phenomenon where microtubules grow and shorten, stochastically switching between these phases of assembly. Dynamic instability is controlled in part by post-translational modifications (PTMs), a set of chemical and biochemical alterations to tubulin subunits that have been incorporated into the microtubule lattice. The dynamic properties of microtubules make them useful for carrying out many cellular processes, including cell division, intracellular trafficking, and polarity establishment.

One of the best characterized microtubule PTMs, tyrosination-detyrosination (Y/ΔY) of α-tubulin, is an important regulatory signal for mitosis, neuronal physiology, and muscle mechanotransduction. Consequently, abnormal tyrosination levels are associated with cell transformation and tumor aggressiveness, heart failure, and cardiomyopathies. Although several proteins have been identified that can discriminate between Y- and ΔY microtubules, the underlying mechanisms and the full repertoire of MAPs that can read the Y/ΔY code remained unknown, and a complete understanding of the cycle is still lacking. Accordingly, there is a need to identify Y- and ΔY-microtubules to understand, monitor, and modulate the nature of microtubule dynamics as well as to identify interventions that can regulate such processes.

### SUMMARY

The present disclosure relates to labeling of proteins for evaluating changes in microtubule dynamics. Included are compositions and methods that permit detection of shortening and/or lengthening of microtubules for research, diagnostic, and therapeutic purposes.

In some embodiments, provided herein are methods for evaluating microtubules. For example, in some embodiments, provided herein are methods for evaluating dynamic changes in microtubule structure (e.g., changes in length). In particular, methods are provided that permit the detection of microtubule shortening. In some embodiments, the methods employ an EML protein (e.g., labeled EML protein). In some embodiments, the EML protein is an EML2 protein. In some embodiments, the EML protein is an EML2-S protein. In some embodiments, the EML protein is an EML2-L protein. In some embodiments, the method comprises the step of contacting a sample comprising microtubules with a labeled EML protein, and detecting a shortening of microtube length. The microtubules that are analyzed may be present in any environment, including both in vitro (e.g., in test tube, in culture, etc.) and in vivo environments. In some embodiments, the microtubules are analyzed in a live cell (e.g., in vitro or in vivo).

In some embodiments, the contacting comprises exposing a sample (e.g., a sample comprising one or more cells) to exogenous labeled EML protein such that the labeled protein comes into contact with microtubules within the sample. In some embodiments, the labeled protein is expressed or generated in a cell containing the microtubules to be analyzed or in proximity to a cell containing the microtubules to be analyzed. For example, in some embodiments, the contacting comprises providing an expression vector to a cell that encodes for an EML protein. In some embodiments, the EML protein is fused to a peptide sequence that generates a labeled EML protein when expressed (e.g., an EML-GFP fusion).

Labeled proteins used in the compositions and methods may be labeled with any suitable, detectable label. Such labels include, but are not limited to fluorescent labels (i.e., fluorophores), luminescent labels, stable isotopes, and mass tags. Fluorescent dyes are particularly beneficial when analyzing dynamic changes in microtubule structure. Fluorophores include, but are not limited to, organic dyes (e.g., fluorescein, rhodamine, Texas red, ALEXA dyes, etc.), biological fluorophores (e.g., green fluorescent protein (GFP), R-phycoerythrin), and quantum dots. Proteins can be labeled chemically or biologically (e.g., enzymatically). In some embodiments, chemical methods of protein labeling involve the covalent attachment of the label to amino acids using a label conjugated to chemical groups that react with specific amino acids. In some embodiments, a protein tag is attached (e.g., recombinantly) to the protein of interest. The protein tag may itself be detectable or may facilitate detection via attachment of a label to the protein tag or through affinity binding (e.g., to an antibody, surface, etc.). In some embodiments, the protein tag comprises one or more of: ALFA-TAG, AviTag, C-tag, Calmodulin-tag, iCAPTAG, polyglutamate tag, polyarginine tag, E-tag, FLAG-TAG, HA-tag, His-tag, Myc, tag, NE-tag, NE-tag, Rho1D4-tag, S-tag, SBP-tag, SOFTAG 1, Spot-tag, Strep-tag, T7-tag, TC tag, Ty tag, V5 tag, VSV-tag, Xpress tag, SPYTAG, BCCP, Glutathione-S-transferase tag, GFP-tag, HALOTAG, mCherry, SNAP-TAG, CLIP-TAG, HUH-tag, maltose binding protein-tag, Nus-tag, thioredoxin-tag, PA-tag, Fc-tag, CRDSAT-tag, or HiBiT-tag. In some embodiments, a fluorescent dye associates with the protein tag to label the protein of interest.

In some embodiments, the methods utilize a second protein that identifies lengthening of microtubules. As such, both shortening and lengthening can be monitored. In some such embodiments, the method comprises contacted microtubules with an agent that identifies lengthening of microtubules. Such agents include tip tracking proteins such as EB1 (aka MAPRE1, microtubule associated protein PR/EB family member 1), EB3 (aka MAPRE3, microtubule-associated protein PR/EB family member 3), or CLIP-170 (aka Cytoplasmic linker protein CLIP-170) that track a growing microtubule end. In some embodiments, the EML protein is labeled with a first label and the second protein is labeled with a second, different label. The labels may be selected such that dynamic changes in microtubule lengthening and shortening can be readily distinguished from on other. For example, a first label may be selected to generate a first detectable color (e.g., green) and a second label may be selected to generate a second, distinguishable, color (e.g., red).

The methods may be employed for any desired use. In some embodiments, the methods are used for research to study microtubule function and/or the role microtubules play in biological processes, including at the molecular, cellular, tissue, or organism levels. In some embodiments, the methods are used for diagnostic purposes, to diagnose a disease or condition. In some embodiments, the methods are used for therapeutic purposes: for example, to identify, test, or monitor therapeutic agents.

In some embodiments, provided herein are methods for analyzing an agent, comprising: exposing a sample comprising microtubules to the agent and to a labeled EML protein, and determining an ability of the agent to alter microtubule length. In some embodiments, the agent is a drug (e.g., small molecule drug, peptide, protein, nucleic acid, or the like). In some embodiments, the agent is a candidate drug that is tested for efficacy or safety. In some embodiments, the agent is a known therapeutic drug. In some embodiments, the testing is conducted to determine an impact of the drug on microtubule structure or function to address a disease, condition, or metabolic or cellular status that is the result of microtubule structure or function. In some embodiments, the testing is conducted to determine an impact of the drug on microtubule structure or function that results as a side-effect of the drug.

Also provided herein are compositions. For example, in some embodiments, the composition comprises one or more components useful, necessary, or sufficient for practicing one or more of the methods described herein. Compositions include, but are not limited to, systems, kits, reagents, reactions mixtures, and the like. For example, in some embodiments, the compositions comprise an EML protein (e.g., EML2). In some embodiments, the compositions further comprise one or more detectable labels. In some embodiments, the label is attached (e.g., covalently attached) to the EML protein. In some embodiments, the compositions comprise a second protein (e.g., EB1, EB3, CLIP-170). In some embodiments, the second protein comprises a label. In some embodiments, the second protein comprise a different label than a label on the EML protein. In some embodiments, the second protein comprises the same label as the label on the EML protein. In some embodiments, the compositions comprise one or more expression vectors encoding an EML protein (e.g., EML2) and/or a second protein (e.g., EB1, EB3, CLIP-170). In some embodiments, a single vector encodes both the EML protein and the second protein. In some embodiments, the vector further encodes one or more detectable labels (e.g., fused to the EML protein and/or second protein). For example, an all-in-one packaging of microtubule end trackers using an internal ribosome entry site may have both EB3 with mCherry labeling and EML2-L with mNeonGreen labeling in a single vector (see e.g., FIG. 18; SEQ ID NO:7).

In some embodiments, the composition is a kit. In some embodiments, the kit provides one or more reagents or other components (e.g., instructions, storage or reaction vessels, software, positive and/or negative controls, cells, instrumentation (e.g., detection instruments)) that find use in the methods described herein. Typically, a kit provides packaging that permits storage and/or shipment of the kit components, for example from a location of manufacture to a location of use. Reagents provided in the kit include, but are not limited to, one or more or each of an EML protein (e.g., EML2), a second protein (e.g., EB1, EB3, CLIP-170), detectable labels, storage buffers, reaction buffers, an expression vector, and cell culture components.

In some embodiments, the composition is a reaction mixture. In some embodiments, the reaction mixture comprises an EML protein (e.g., EML2), a label (e.g., bound to EML2), and a sample comprising microtubules (e.g., a sample comprising one or more cells having microtubules). In some embodiments, a labeled EML2 protein is in contact with a microtubule within a live cell. In some embodiments, the microtubule is decreasing in length. In some embodiments, a second protein (e.g., EB1, EB3, CLIP-170) is also in contact with a microtubule within the live cell. In some embodiments, the second protein comprises a second, different label.

In some embodiments, the composition is a cell that is altered from its natural state to include a heterologous EML protein (e.g., EML2). In some embodiments, the cell further comprises a second protein (e.g., EB1, EB3, CLIP-170). In some embodiments, the cell comprises an expression vector that encodes an EML protein (e.g., EML2) and/or a second protein (e.g., EB1, EB3, CLIP-170). In some embodiments, the cell comprises a genomic or other native sequence modified to express a heterologous EML protein (e.g., EML2) and/or a second protein (e.g., EB1, EB3, CLIP-170). In some such embodiments, the EML protein and/or second protein may be labeled.

In some embodiments, an instrument system and/or software is provided that allows a user to visualize shortening of microtubules via a labeled EML protein (e.g., labeled EML2 protein) and/or a labeled second protein (e.g., EB1, EB3, CLIP-170). In some embodiments, the instrument comprises one or more detectors (e.g., comprising a lens component and a camera component) for detecting labels at a cellular and/or molecular scale. In some embodiments, the instrument comprises a display for displaying an image or representation of microtubules that are undergoing a structural change. In some embodiments, the display shows two or more different colors or contrasts that represent and differentiate a microtubule that is growing from one that is shrinking. In some embodiments, software is provided to translate data collected from a detector into information that is displayed to the user. In some embodiments, a numerical is provided to the user. For example, in some embodiments, the numerical display provides information that reveals the number of, degree of, relative percent of, or other metric of microtubules that are shortening and/or lengthening. In some embodiments, the software generates a result that is conveyed to the user. The result may be a diagnostic or therapeutic outcome result (e.g., a yes/no answer or other qualitative or quantitative answer that reveals whether a cell, tissue, or subject has a particular status (e.g., disease or condition) or whether an agent has a particular impact).

### DEFINITIONS

As used herein, the term “EML protein” (aka EMAP Like; Echinoderm Microtubule Associate Protein Like) refers to a family of proteins with microtubule binding activity, including EML1, 2, 3, 4, 5, and 6. “EML2 protein” (aka EMAP Like 2; EMAP-2; EMAP2; ELP70; Echinoderm MT-Associated Protein (EMAP)-Like Protein 70; Microtubule-Associated Protein Like Echinoderm EMAP; Echinoderm Microtubule Associated Protein Like 2; Echinoderm Microtubule-Associated Protein-Like 2; EMAPL2) refers to a microtubule binding protein within the EML protein family. Human EML2, in its natural form, is found on chromosome 19q13.32 (full-length, wild-type sequence at GenBank AAD19904.1; SEQ ID NO:1). Additional EML2 proteins include those from cow (XM_003587274.2), dog (XM_003432569.2), mouse (NM_001162996.1), rat (NM_138921.1), and opossum. Unless specified otherwise, the term “EML2” includes functional fragments and variants of an EML2 protein. With respect to the present disclosure, functional fragments and variants are those that maintain the ability to bind to microtubules to facilitate detection of microtubule shortening, even if one or more other properties associated with a corresponding natural EML2 protein is lost.

As used herein, the term “EB protein” encompasses a group of conserved microtubule plus-end tracking proteins, their homologs, orthologs, and variants. There are three main EB proteins in the mammalian species, EB1, EB2, and EB3. The proteins encoded by the MAPRE family are encompassed within the “EB proteins.” For example, the amino acid sequences for human and rodent EB proteins and known variants can be found at the NCBT worldwide website (ncbi.nlm.nih.gov).

The term “heterologous expression,” as used herein, refers to the protein expression of a gene, a nucleic acid or a cDNA, which is foreign to the organism in which the expression occurs. A “heterologous protein” is a non-native protein that is present in a cell, whether expressed in the cell or exogenously added to the cell. A heterologous protein may be a synthetic protein or may be a natural protein from one source (e.g., a first cell) that is added to another source (e.g., a second cell).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine.

### DESCRIPTION OF FIGURES

FIGS. 1A-H shows quantitative proteomics identifying candidate readers of Y/ΔY-microtubules. FIG. 1A shows schematic of the Y/ΔY reader screen. FIG. 1B shows immunoblot analysis for tubulin Y/ΔY state in the microtubule pellet fractions prepared from wild-type and HeLa cells over-expressing VASH1-SVBP. FIG. 1C shows SDS-PAGE analysis of microtubule pellet fractions. Arrowhead indicates tubulin. FIG. 1D shows volcano plot depicting proteins co-sedimented with Y- or ΔY-microtubules shown in magenta or green background, respectively. X axis represents log 2 relative abundance of each protein in VASH1 OE over wild-type samples (VASH/WT). Y axis shows −log 10 p-value. Blue dots highlight CAP-Gly proteins. Magenta and green dots represent candidate Y- and ΔY-readers, respectively. FIGS. 1E-G show immunoblot analysis of CAP-Gly proteins (FIG. 1E), Y-reader candidate (FIG. 1F) and ΔY-reader candidates (FIG. 1G) in microtubule pellet fractions. Relative intensity of the bands of VASH1 OE over WT samples (normalized against corresponding total α-tubulin band intensity) is shown next to each blot. FIG. 1H shows comparison of TMT- and immunoblot-based quantitation of relative protein abundance in microtubule pellet fractions. Background color indicates Y-readers (magenta) and ΔY-readers (green) based on the TMT analysis.

FIGS. 2A-H show that EML2-S is a Y-αCTT reader. FIG. 2A shows domain organization of human EML proteins (Coiled-coil (CC), basic and disordered region; Hydrophobic Echinoderm-MAP like protein motif (HELP)). FIG. 2B shows volcano plot of TMT analysis highlighting EML1, 2, 3 and 4. FIG. 2C shows immunoblot microtubule pellet fractions prepared from lysates of HeLa cells overexpressing PA-tagged EML1, 2-L, 2-S, 3 and 4 treated without (Y-conditions) or with (ΔY-conditions) CPA. FIGS. 2D-E show immunofluorescence staining of PA-EML2-L and PA-EML2-S in interphase HeLa cells (FIG. 2D) or cells undergoing cytokinesis (FIG. 2E). In FIG. 2D, boxed regions in the PA-EML2 channel are enlarged, and line profiles are analyzed along the yellow lines in the merge channels. In FIG. 2E, line profiles are analyzed on the white lines placed along midbody microtubules as shown in the inserted images on the line profiles. ΔY-α-tubulin is not shown in the merged images in FIG. 2E. Scale bars, 20 μm (whole cell images in FIG. 2D) and 5 μm (enlarged images in FIG. 2D and FIG. 2F) FIG. 2F shows box plots of relative abundance of PA-EML2 normalized against tubulin in the midbody microtubules over the entire cell area. The box indicates 75th, 50th and 25th percentile. Whiskers and an outlier (shown by a dot) are plotted by the Tukey method. FIG. 2G shows microtubule co-pelleting assay using purified His-EML2-S and Y- or ΔY-HeLa microtubules. P, pellet; S, supernatant. The ratios of His-EML2-S between pellet and supernatant fractions are quantified and shown on the Coomassie-gel. FIG. 2H shows quantification of His-EML2-S in the pellet fractions in FIG. 2G. After baseline (No MTs) subtraction, fractions (%) of His-EML2-S bound to microtubules are plotted with SD (n=3).

FIGS. 3A-G show the structural study of the Y-CTT recognition motif of the EML2-S TAPE domain. FIG. 3A shows the electrostatic potential of EML2-S. Potential contours are shown for +5/−5 kT/e in blue and red respectively. FIG. 3B shows molecular details of the positive electrostatic patch in the N-terminal s-propeller. Blue is basic residues; green is aromatic, and white is hydrophobic. Residue numbers are for EML2-S. FIG. 3C shows schematic of EML2-S residues targeted for mutagenesis of the R-patch and hydrophobic clamp. Residues in the R-patch and the hydrophobic clamp are shown in blue and green, respectively. FIG. 3D shows immunofluorescent staining of HeLa cells overexpressing EML2-S mutant proteins. Scale bars, 20 μm (whole cell images) and 5 μm (enlarged images). FIG. 3E shows box plots of a colocalization metrics, threshold overlap score (TOS) between EML2-S mutants and microtubules in HeLa cells. The box indicates 75th, 50th and 25th percentile. Whiskers and outliers (shown by dots) are plotted by the Tukey method. FIG. 3F shows In vitro microtubule co-pelleting assay using His-EML2-S_{R69E} mutant and Y/ΔY-HeLa microtubules. EML2-S band intensity was quantified and shown on the Coomassie gel. FIG. 3G shows model of EML2-S:Y-CTT binding. A fast but unstable electrostatic interaction between the glutamate residues in the CTT and the R-patch occurs first, and binding is then stabilized by hydrophobic interaction with α-tubulin's C-terminal tyrosine.

FIGS. 4A-G shows that EML2 increases microtubule lifetimes by increasing rescue and slowing shrinkage. FIG. 4A shows time lapse images and kymograph of mNG-EML2-L enriched at shrinking microtubule ends in HeLa cells. Arrowheads mark the position of shrinking microtubule ends. Vertical bar, 30 s; horizontal bar, 2 μm. FIG. 4B shows time lapse images and kymograph of mNG-EML2-L and EB3-mCherry co-expressed in HeLa cells. Arrowheads mark the position of microtubule ends. Rescue [R] and catastrophe [C] are indicated. Vertical bar, 30 s; horizontal bars, 2 μm. FIG. 4C shows In vitro microtubule dynamics assay with lysates prepared from COS7 cells expressing mNG-EML2-L. Cyan, microtubule seeds; magenta, dynamic microtubules; green, mNG-EML2-L. Vertical bars, 1 min; horizontal bars, 2 μm. Arrowheads indicate enrichment of EML2-L at a shrinking microtubule end. FIG. 4D shows quantification of the microtubule shrinkage rate. n, number of shrinkage events. FIG. 4E shows immunofluorescent staining of PA-EML2-L and S in nocodazole-treated HeLa cells. Boxed regions were enlarged and shown with line profiles analyzed along each microtubule. Arrowheads indicate EML2 enriched at microtubule ends. Magenta, PA-tagged EML2 (L/S); green, α-tubulin; blue, DNA. Bars, 5 μm (whole cell images) and 1 μm (enlarged images). FIG. 4F shows In vitro microtubule dynamics assay using a constant concentration of brain tubulin (7 μM) with or without purified His-EML2-S. Seeds are shown in magenta and dynamic microtubules in green. Vertical bars, 3 min; horizontal bars, 3 μm. FIG. 4G shows measurements of dynamics parameters. Mean+/−SD (from 3-5 independent experiments).

FIG. 5 shows a comparison of the levels of α-tubulin detyrosination in HeLa WT and VASH1/SVBP overexpressing cell lines. (A,B) show immunoblot analysis for tubulin Y/ΔY state in lysates (A) and microtubule pellet fractions (B) prepared from WT HeLa and HeLa cells overexpressing VASH1-SVBP (VASH1 OE). After the Taxol-induced microtubule assembly, ΔY-tubulin decreased to an undetectable level in the VASH1 OE conditions.

FIGS. 6A-B show uncropped immunoblot images for candidate readers. Molecular weight indicated for each protein is based on the canonical sequence reported on Uniport. Bands shown with asterisks are more or less different from expected positions.

FIGS. 7A-E show GST-CTT pulldown experiment for identification of CTT binding Y- and ΔY-readers. FIG. 7A shows GST-CTT constructs (SEQ ID NOS:8-10). FIG. 7B shows purified GST and GST-CTT proteins are analyzed on a gel. An arrowhead indicates the position of GST and GST-CTT proteins. FIG. 7C shows proteins pulled down with GST, GST-CTT or GST-CTT^{ΔY} in buffer (control) or CHL-1 cell lysates were analyzed on a gradient gel. Asterisks indicate proteins detected specifically in the GST-CTT^{Y} pulldown. FIG. 7D shows venn diagram of MS analysis. CTT binding proteins were defined as proteins detected in GST-CTT (Y/ΔY) pulldown but are absent in the control GST pulldown. Among them, GST-CTT^{Y}- and GST-CTT^{ΔY}-specific proteins were referred to as Y-CTT and ΔY-CTTreaders, respectively. FIG. 7E shows CTT binding proteins identified by LC-MS/MS. MAPs that were identified with minimum 2 unique peptides are shown in the table.

FIGS. 8A-C show detection of EML2 isoforms in fractions co-sedimented with Y- and ΔY-microtubules. FIG. 8A shows EML2 peptides identified in the MS analysis. Peptides derived from longer isoforms (2 and 3) are depicted with blue bars whereas peptides that are common to any isoforms are shown in orange. Relative abundance of these peptides are averages and shown. FIG. 8B shows comparison between western blot based- and MS-based calculations of relative abundance of EML2 isoforms. Densitometric analysis was normalized against corresponding total α-tubulin. FIG. 8C shows EML2 peptides identified in the TMT analysis (SEQ ID NOS: 11-42).

FIG. 9 shows alternative method of Y/ΔY microtubule preparations using carboxypeptidase A (CPA)-mediated in vitro detyrosination. HeLa lysate was treated with CPA prior to the microtubule assembly. Microtubule pellet fractions in untreated (Ctrl) or CPA-treated (CPA) lysates were analyzed with immunoblot. Y/ΔY binary conditions that were comparable to the WT/VASH1 OE method, were generated. EML2-S was not detected in the microtubule pellet prepared from CPA-treated lysate.

FIG. 10 shows live imaging of HeLa cells transiently expressing EGFP (N-/C-terminal) or SNAP-tagged EML2-L/S. HeLa cells were transfected with corresponding constructs and imaged. EML2-S did not exhibit microtubule localization when fused to bulky tags. Scale bars, 10 μm.

FIG. 11 shows immunoblot analysis for lysates prepared from HeLa cells transiently expressing PA-EML2-L or S. Lysates were prepared from HeLa cells transfected with PA-EML2 constructs and analyzed with immunoblot analysis. A total of 15 μg of proteins was loaded in each lane. PA-EML2 overexpression did not alter the Y/ΔY state in cells.

FIGS. 12A-B shows purification of His-EML2-S from insect cells and Y-/ΔY-tubulin from HeLa cells. FIG. 12A shows purification of His-EML2-S. His-EML2-S purified by 2 steps: Ni-NTA affinity chromatography (left) followed by size exclusion chromatography (right). Arrowheads indicate the position of His-EML2-S on SDS-PAGE gels. A representative elution profile is shown in the middle with an arrow indicating the peak fractions corresponding to His-EML2-S. FIG. 12B shows purification of Y- and ΔY-tubulin from HeLa cells using TOG affinity chromatography. A workflow (left) and images of a gel and blots for representative preps (right) are shown. Prior to the affinity purification step, HeLa lysates were treated with or without CPA for ΔY or Y-tubulin preps, respectively.

FIGS. 13A-B show transient expression of PA-EML2-S mutants in HeLa cells. FIG. 13A shows immunoblot analysis of lysates prepared from HeLa cells transiently expressing PA-EML2-S mutants. A total of 15 μg of proteins was loaded in each lane. Comparable levels of PA-EML2-S proteins were detected. The Y/ΔY state was not affected by overexpression of PA-EML2 mutants. FIG. 13B shows quantification of average intensities of EML2-S mutants and microtubules in the immunostained HeLa cells. Fluorescent intensities of PA-EML2-S mutants as well as microtubules were comparable among different mutants. The box and line indicate 75th and 25th percentile, and median, respectively. Whiskers and outliers (shown by dots) are plotted by the Tukey method.

FIG. 14 shows additional microtubule dynamics parameters measured in the in vitro assay using purified His-EML2-S protein. (A) shows alternate rescue frequency described in number of events per microtubule length (events/μm). (B) shows cumulative probability of microtubule nucleation. His-EML2-S shows minimum impact on the microtubule nucleation. (C) shows representative TIRF images of microtubules at 15 min and measurements of microtubule length. Magenta, seeds; green, elongated microtubules. Scale bar, 6 μm.

FIGS. 15A-B show sequence alignments of EML1-4 TAPE domain. FIG. 15A shows conserved residues in the R-patch (blue) and the hydrophobic clamp (green/white) were mapped in the ribbon diagram (upper) and indicated in the aligned sequence (lower). FIG. 15B shows multiple sequence alignment was performed using ClustalX 2.1.

FIG. 16 shows an exemplary variant EML2-L protein containing GCN4 domain and images comparing microtubule detection with and without the variant sequence.

FIGS. 17A-B show an exemplary variant EML2-L protein (EML2-L AAAA) and images comparing microtubule detection with and without the variant sequence.

FIG. 18 shows a cloning strategy and an expression vector providing an all-in-one vector containing both an EML2-L protein fused to a mNeonGreen label and an EB3 protein fused to an mCherry label.

### DETAILED DESCRIPTION

### Microtubules

Microtubules are formed by heterodimers of two globular proteins called α- and β-tubulin, which assemble into rod-shaped filaments. Microtubules form cytoplasmic networks and serve as frameworks of important organelles, including the mitotic spindle, centrioles, cilia and bundles inside neurites. Microtubules are involved in a vast array of cellular functions, including cell shape, motility and division, intracellular signaling and transport, cell differentiation, and generation of specific organelles. They control intracellular trafficking of proteins, organelles and vesicles, and separate chromosomes during mitosis, and are fundamental for cell proliferation, secretory processes and vascularization. In addition to microtubule-only functions, actin-tubulin interactions control wound healing, T-cell immune response and growth of neuronal cones.

Microtubules are highly dynamic, in which the ends of individual polymers transition between growing and shortening in vivo and in vitro. These microtubule dynamics are mediated by interactions between microtubules and a cohort of molecular motors and microtubule-associated proteins (MAPs) through post-translational modifications (PTMs). One of the best characterized microtubule PTMs is tyrosination-detyrosination cycle at the α-tubulin C-terminal site. During detyrosination, a genetically encoded C-terminal tyrosine residue is enzymatically removed by vasohibin-SVBP complexes or MATCAP. Detyrosination can be reversed by tubulin tyrosine ligase (TTL), creating a Y/ΔY cycle. Newly polymerized microtubules are mainly tyrosinated while stable microtubules are detyrosinated. In proliferating cells, microtubules are globally dynamic and predominantly tyrosinated, while in neurons, microtubules are generally stable and detyrosinated outside major neuronal structures such as growth cones and dendritic spines which remain highly dynamic and tyrosinated

The detyrosination-tyrosination cycle of α-tubulin and its dysregulation have been linked to several cellular, physiological, and pathological processes. Examples of pathologies involving the TCP include neurological disorders, cardiovascular diseases, cancer, and others.

### Neurological Disorders

Microtubules play crucial role in maintaining axonal integrity and forming the tracks for axonal transport and axonal transport defects are a direct cause of neurodegenerative disease. Reversible detyrosination of α-tubulin is crucial to microtubule dynamics and functions, such as axonal transport, and in dendritic spines. Modifications of microtubule dynamics have been observed in several neurodegenerative diseases such as Alzheimer's disease, Parkinson's disease, Amyotrophic Lateral Sclerosis, and tauopathies like Progressive Supranuclear Palsy. Studies also suggest that microtubule dynamics in the adult brain function is required for learning and memory and may be compromised in degenerative diseases. For example, studies show that TTL expression is decreased, whereas detyrosinated tubulin is increased in Alzheimer's disease patient brains.

### Cardiovascular

Proper levels of detyrosinated microtubules in cardiomyocytes provide the necessary mechanical resistance and stiffness for functional contractility. Abnormally high levels of detyrosinated microtubules impair the contractility of cardiomyocytes.

Additionally, myocardial infarction, the main cause of ischaemic heart disease (IHD) and chronic heart failure, is a serious ischaemic syndrome in which the blood supply to the heart is blocked, thus causing substantial myocardial cell death and loss of function in the remaining viable cells. Microtubule (MT) detyrosination, which is associated with DESMIN at force-generating sarcomeres, is upregulated in the failing hearts of patients with ischaemic cardiomyopathy and hypertrophic cardiomyopathies, and suppression of microtubule detyrosination improves contractility in failing cardiomyocytes. VASH1 or VASH2, coupled with a small vasohibin-binding protein (SVBP), forms a Tubulin Carboxy Peptidase (TCP) that is capable of tubulin detyrosination. MATCAP, a newly identified TCP, may also participate in tubulin detyrosination in cardiomyocytes. Depletion of VASH1 speeds contraction and relaxation in failing human cardiomyocytes. Structural and biophysical studies have suggested that VASH interacts with the C-terminal tail of α-tubulin.

### Cancer

Misregulation of tyrosination/detyrosination cycle of tubulin, frequently observed during cancer progression, is associated with increased tumor aggressiveness. This is because the carboxyl-terminus of α-tubulin is involved in the control of cell cycle progression and in the association/dissociation of motor proteins during cell division.

Considering the key role of microtubules for various cellular processes, it is not surprising that microtubule-targeting compounds such as paclitaxel (Taxol) are among most widely used drugs in anti-cancer therapy. The chemotherapeutic drug Taxol is known to interact within a specific site on β-tubulin and as such stabilizes microtubules and impacts primarily dividing cells. However, microtubule-targeting drugs suffer from several drawbacks such as high toxicity and eventual development of drug-resistant cancer cells. For that reason, it is important to continue developing compounds that impact microtubule functions.

In addition to the above, disorders involving microtubule detyrosination include muscular dystrophies, vascular disorders, retinal degeneration, infertility and/or ciliopathies.

Experiments conducted during the development of the invention identified EML proteins as capable of associating with and detecting shortening microtubules. EML proteins, or variants or fragments thereof, find use in the detection and analysis of microtubule shortening. Such proteins may be combined with agents that identify microtubule lengthening (e.g., EB1, EB3, CLIP-170 proteins) to provide the first comprehensive system for analyzing the dynamic changes in microtubule structure.

### EML Proteins

In some embodiments, an EML proteins is used to analyze microtubule structure and dynamic changes to microtubule structure. Echinoderm microtubule-associated protein like (EML) is a family of MAPs, with 6 currently identified members (i.e., EML1-6) found in mammals. For example, the wild-type EML2 sequences from several mammalian species are provided below.

Human
(SEQ ID NO: 1)
MSSFGAGKTKEVIFSVEDGSVKMFLRGRPVPMMIPDELAPTYSLDTRSELPSCRLKLEW
VYGYRGRDCRANLYLLPTGEIVYFVASVAVLYSVEEQRQRHYLGHNDDIKCLAIHPDM
VTIATGQVAGTTKEGKPLPPHVRIWDSVSLSTLHVLGLGVFDRAVCCVGFSKSNGGNLL
CAVDESNDHMLSVWDWAKETKVVDVKCSNEAVLVATFHPTDPTVLITCGKSHIYFWTL
EGGSLSKRQGLFEKHEKPKYVLCVTFLEGGDVVTGDSGGNLYVWGKGGNRITQAVLG
AHDGGVFGLCALRDGTLVSGGGRDRRVVLWGSDYSKLQEVEVPEDFGPVRTVAEGHG
DTLYVGTTRNSILQGSVHTGFSLLVQGHVEELWGLATHPSRAQFVTCGQDKLVHLWSS
DSHQPLWSRIIEDPARSAGFHPSGSVLAVGTVTGRWLLLDTETHDLVAIHTDGNEQISVV
SFSPDGAYLAVGSHDNLVYVYTVDQGGRKVSRLGKCSGHSSFITHLDWAQDSSCFVTN
SGDYEILYWDPATCKQITSADAVRNMEWATATCVLGFGVFGIWSEGADGTDINAVARS
HDGKLLASADDFGKVHLFSYPCCQPRALSHKYGGHSSHVTNVAFLWDDSMALTTGGK
DTSVLQWRVV
Dog
(SEQ ID NO: 2)
MSESGGDFLPLGALFLAHLPASQAAADDNLSGTSGMEVDDRVSALEQRLQLQEDELAV
LKAALADALRRLRACEEQGAALRARGTPKGRAPPRLGTTASVCQLLKGLPTRTPLNGA
GPPRRVGGYATSPSSPKKEASSGRSARRYLSPERLASVRREDPRSRTTSSSSNCSAKKEG
KTKEVIFSLEEGSVKMFLRGRPVPMLIPDEMAPTYSLDTRSELPPSRLKLDWVYGYRGR
DCRANLYLLPTGEIVYFVASVAVLYSVEEQRQRHYLGHNDDIKCLAVHPDMVTIATGQ
VAGTTKEGKPLPPHVRIWDSVSLSTLHVLGLGVFDRAVCCVGFSKSNGGNLLCAVDES
NDHMLSVWDWAKEAKVVDGKKHEKPKYVLCVTFLEGGDVVTGDSGGNLYVWGKGG
NRITQAVLGAHDGGVFGLCALRDGTLVSGGGRDRRVVLWGSDYSKLQEVEVPEDFGP
VRTVAEGRGDTLYVGTTRNSILQGSVHTGFSLLVQGHVEELWGLATHPSRAQFVTCGQ
DKLVHLWSAESHQPLWSRIIEDPARSAGFHPSGSVLAVGTVTGRWLLLDTETHDLVAIH
TDGNEQISVVSFSPDGAYLTVGSHDNLVYVYTVDQGGRKVSRLGKCSGHSSFITHLDW
AQDSSCFVTNSGDYEILYWDPATCKQITSADAVRNVEWATATCVLGFGVFGIWSEGAD
GTDINAVARSHDGKLLASADDFGKVHLFSYPCCQPRALSHKYGGHSSHVTNVAFLWDD
SMALTTGGKDTSVLQWRVV
Mouse
(SEQ ID NO: 3)
MSSFGIGKTKEVIFSMEEGSVKMFLRGRPVPMLIPDELAPTYSLDTRSELPSSRLKLDWV
YGYRGRDCRANLYLLPTGEVVYFVASVAVLYSVEEQRQRHYLGHNDDIKCLAVHPDM
VTIATGQVAGTTKEGKPLPPHVRVWDSVSLSTLHVLGLGVFDRAVCCVAFSKSNGGNL
LCAVDESNDHVLSVWDWAKESKVVDSKCSNEAVLVATFHPTDPSLLITCGKSHIYFWS
LEGGSLSKRQGLFEKHEKPKYVLCVTFLEGGDVVTGDSGGNLYVWGKGGNRITQEVQG
AHDGGVFALCALRDGTLVSGGGRDRRVVLWGSDYSKVQEVEVPEDFGPVRTVAEGRG
DTLYVGTTRNSILLGSVHTGFSLLVQGHVEELWGLATHPSRAQFVTCGQDKLVHLWSSE
THQPVWSRSIEDPARSAGFHPSGSVLAVGTVTGRWLLLDTETHDLVAIHTDGNEQISVV
SFSPDGAYLAVGSHDNLVYVYTVDQGGRKVSRLGKCSGHSSFITHLDWAQDSTCFVTN
SGDYEILYWDPVTCKQITSADTVRNVEWATATCVLGFGVFGIWPEGADGTDINAVARS
HDGKLLVSADDFGKVHLFSYPCCQPRALSHKYGGHSSHVTNVAFLWDDSMALTTGGK
DTSVLQWRVA
Rat
(SEQ ID NO: 4)
MSSFGTGKTKEVIFSMEEGSVKMFLRGRPVPMLIPDELAPTYSLDTRSELPSSRLKLDWV
YGYRGRDCRANLYLLPTGEVVYFVASVAVLYSVEEQRQRHYLGHNDDIKCLAVHPDM
VTIATGQVAGTTKEGKPLPPHVRVWDSVSLSTLHVLGLGVFDRAVCCVAFSKSNGGNL
LCAVDESNDHVLSVWDWAKESKVVDSKCSNEAVLVATFHPTDPNLLITCGKSHIYFWS
LEGGNLSKRQGLFEKHEKPKYVLCVTFLEGGDVVTGDSGGNLYVWGKGGNRITQEVL
GAHDGGVFALCALRDGTLVSGGGRDRRVVLWGSDYSKVQEVEVPEDFGPVRTVAEGR
GDTLYVGTTRNSILLGSVHTGFSLLVQGHVEELWGLATHPSRAQFVSCGQDKLVHLWS
SETHQPVWSRSIEDPARSAGFHPSGSVLAVGTVTGRWLLLDTDTRDLVAIHTDGNEQIS
VVSFSPDGAYLAVGSHDNLVYVYTVDQGGRKVSRLGKCSGHSSFITHLDWAQDSTCFV
TNSGDYEILYWDAATCKQITSADTVRNVQWATATCVLGFGVFGIWPEGADGTDINAVA
RSHDGNLLVSADDFGKVHLFSYPCCQPRALSHKYGGHSSHVTNVAFLWDDSMVLTTG
GKDTSVLQWRVA

EML proteins may be produced by purification from natural sources, chemically, or, preferably, recombinantly. In some embodiments, the EML protein is produced recombinantly with one or more additional sequences incorporated on the N-terminal end, the C-terminal end, or internally, to facilitate expression, purification, and/or detection of the EML protein. In some embodiments, the additional sequences comprise one or more protein tags. The additional sequences may be attached to the EML protein via a linker. In some embodiments, the nucleic acid sequence that encodes the EML protein is modified (e.g., codon modified) to enhance expression in a recombinant host cell (e.g., bacterial cell, mammalian cell, plant cell, insect cell, etc.).

In some embodiments, the protein is provided as a monomer. In some embodiments, the protein is provided as a multimer (e.g., a dimer, trimer).

### Other Proteins

As discussed above, in some embodiments, EML proteins are used in combination with proteins that detect microtubule lengthening. Such proteins include EB1, EB3, and CLIP-170. In some embodiments, an expression vector encoding both the EML protein and one or more of the other proteins is provided.

The EML proteins also find use in combination with any other protein or agent that binds to and regulates microtubule structure or function. The use of EML proteins allows for an analysis of the impact of such other proteins or agents on microtubule shortening. For example, the other protein or agent may be a microtubule stabilizer. For example, the other protein or agent may be davunetide (also known as NAP, AL-108) and related compounds that affect microtubule stabilization through interaction with microtubule severing proteins via expression of tau. In some embodiments, the other protein or agent may be epothilone D (also known as BMS-241027). This small molecule microtubule-stabilizing drug prevents the pathogenic mechanism in Alzheimer's disease resulting from the phosphorylated tau. In some embodiments, the other protein or agents is TPI 287. Developed by Cortice Biosciences, this drug falls in the taxane class. The mechanism of action involves microtubule stabilization along with affecting levels of phosphorylated tau. In some embodiments, the other protein or agent is LUCIDITY (TauRx), a tau aggregation inhibitor (TAI).

### Variants and Fragments

The peptides and proteins of this invention can be obtained by various means, such as by chemical synthesis or recombinant production.

Any of the proteins described herein may comprise one or more (e.g., 1, 2, 3, 4, 5, 6, 7, 8, 9, 10, 15, 20, 25, 50, 100, 150, 200, etc.) amino acid substitutions. An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH2 can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

In some embodiments, a protein comprises one or more amino acid substitutions and has an amino acid sequence having at least 70% identity (e.g., at least 75%, at least 80%, at least 85%, at least 90%, at least 93%, at least 95%, at least 98%, or at least 99% identity) to an amino acid sequence of SEQ ID NO:1. In some embodiments, substitutions are in positions other than the positions represented by R69, R314, R316 and/or R341 as described in Example 7 or the equivalent positions in homologous proteins.

In some embodiments, the protein comprises a fragment of a wild-type protein. In some embodiments, the fragment comprises a portion of a protein that retains the properties of binding to dynamically changing microtubules (e.g., binds the CTT of α-tubulin). For example, in some embodiments, the fragment differs from a natural protein by removal of amino acids form the C-terminal or N-terminal regions, or from an interior region. In some embodiments, where the protein is an EML protein, the fragment comprises a TAPE domain. In some embodiments, where the protein is an EML protein, the fragment comprises an N-terminal β-propeller.

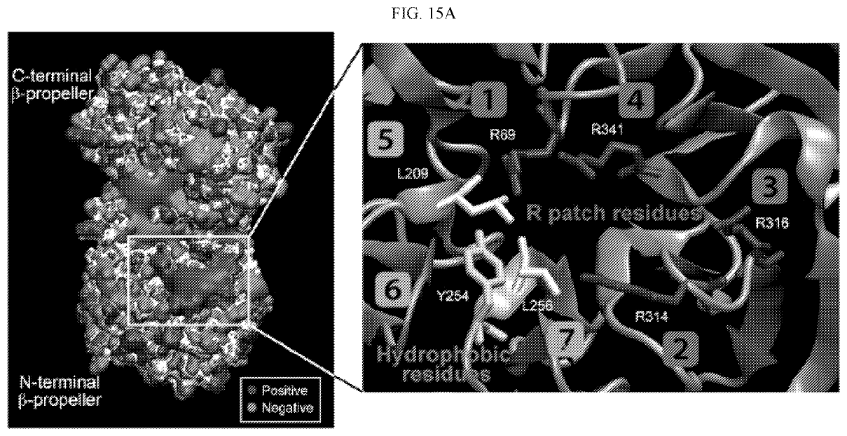

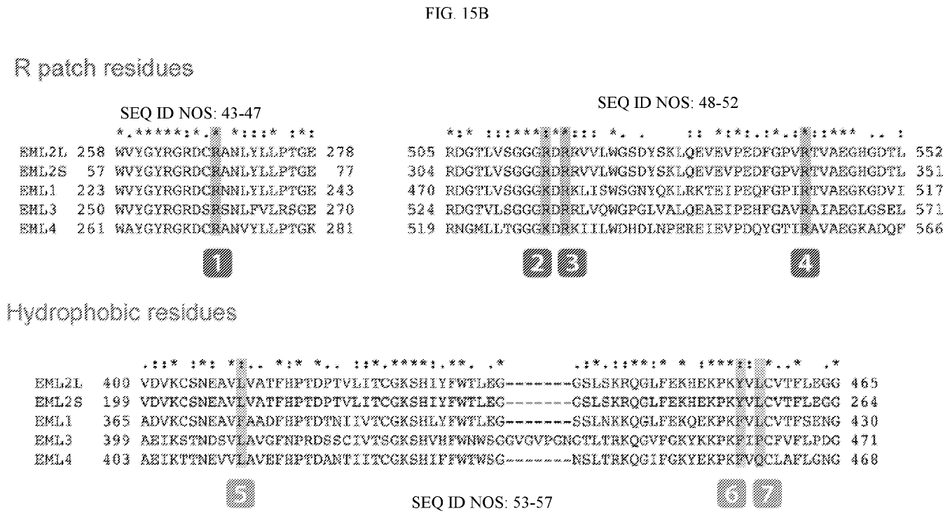

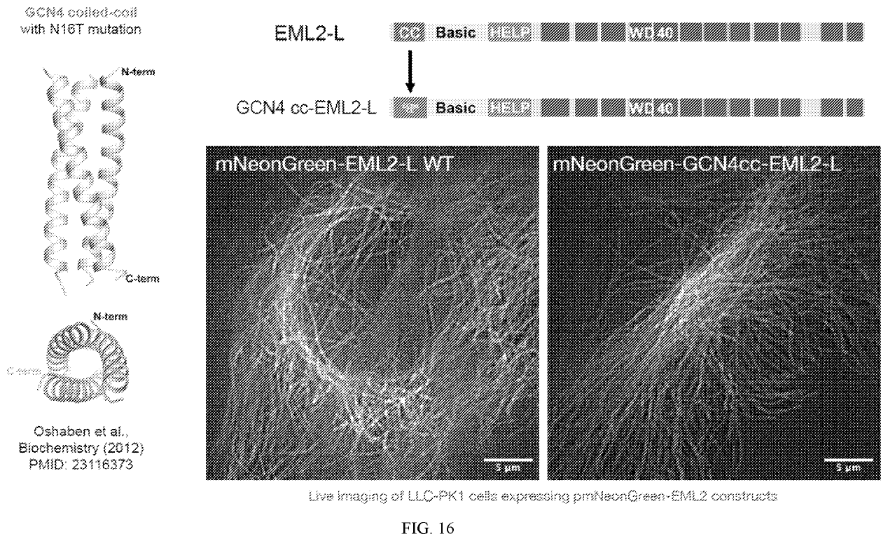

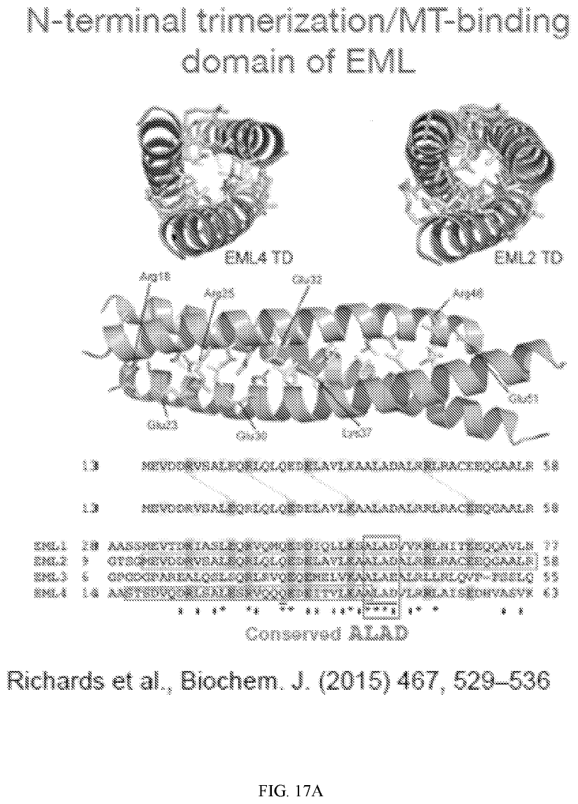

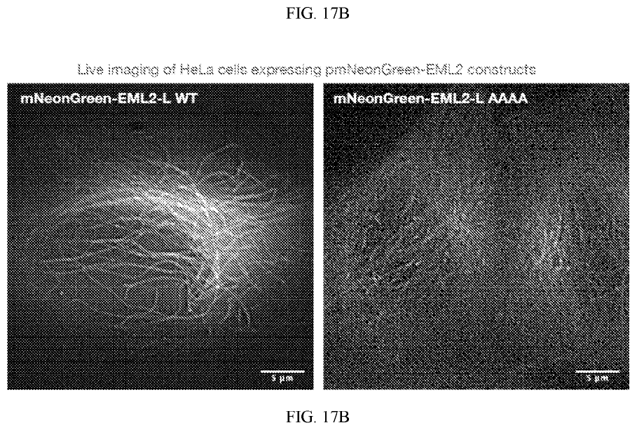

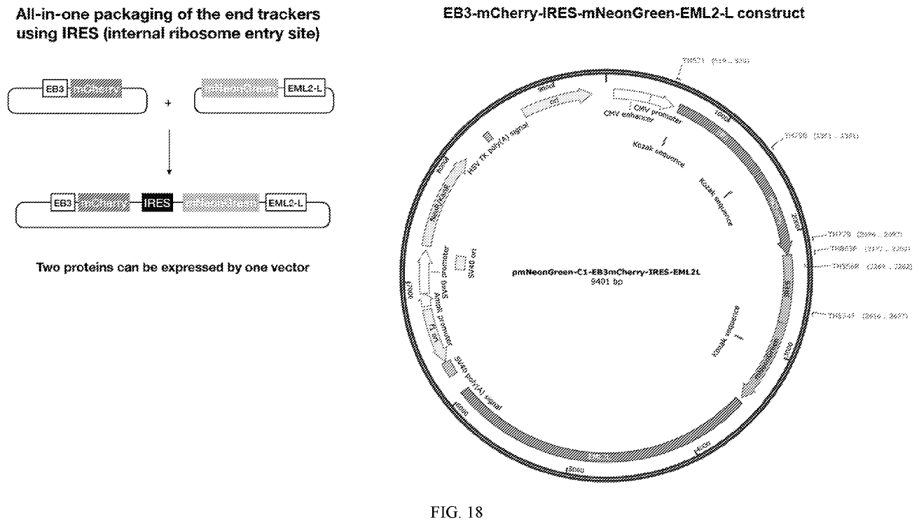

Engineered versions of EML proteins may be used for tracking the microtubules length. For example, proteins with artificial sequences may have leucine zipper trimerization domain of GCN4 replacing EML2-L's N-terminal trimerization/MT binding domain (SEQ ID NO:5). This version of EML2-L tracks shrinking MT plus ends just as wild-type EML2-L does, but does not exhibit other enzymatic activities present in native EML2 that interrupts accurate tracking of microtubules' lengths (see e.g., FIG. 16). Another example of an MT end marker variant is mNG-EML2-L AAAA mutant (SEQ ID NO:6). This version of EML2-L enriches at the microtubule ends and binds less to the lattice, enabling the more accurate tracking of microtubules' lengths (see e.g., FIG. 17).

Variant 1 (GNC4cc-EML2-L)
(SEQ ID NO: 5)
TAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCC
ATTGACGCAAATGGGCGGTAGGCGTGTACGGTGGGAGGTCTATATAAGCAGAGCTG
GTTTAGTGAACCGTCAGATCCGCTAGCGCTACCGGTCGCCACCATGGTGAGCAAGG
GCGAGGAGGATAACATGGCCTCTCTCCCAGCGACACATGAGTTACACATCTTTGGCT
CCATCAACGGTGTGGACTTTGACATGGTGGGTCAGGGCACCGGCAATCCAAATGAT
GGTTATGAGGAGTTAAACCTGAAGTCCACCAAGGGTGACCTCCAGTTCTCCCCCTGG
ATTCTGGTCCCTCATATCGGGTATGGCTTCCATCAGTACCTGCCCTACCCTGACGGG
ATGTCGCCTTTCCAGGCCGCCATGGTAGATGGCTCCGGcTACCAAGTCCATCGCACA
ATGCAGTTTGAAGATGGTGCCTCCCTTACTGTTAACTACCGCTACACCTACGAGGGA
AGCCACATCAAAGGAGAGGCCCAGGTGAAGGGGACTGGTTTCCCTGCTGACGGTCC
TGTGATGACCAACTCGCTGACCGCTGCGGACTGGTGCAGGTCGAAGAAGACTTACC
CCAACGACAAAACCATCATCAGTACCTTTAAGTGGAGTTACACCACTGGAAATGGC
AAGCGCTACCGGAGCACTGCGCGGACCACCTACACCTTTGCCAAGCCAATGGCGGC
TAACTATCTGAAGAACCAGCCGATGTACGTGTTCCGTAAGACGGAGCTCAAGCACTC
CAAGACCGAGCTCAACTTCAAGGAGTGGCAAAAGGCCTTTACCGATGTGATGGGCA
TGGACGAGCTGTACAAGTCCGGACTCAGATCTCGAGCTCAAGCTTCGATGCTGGAAC
GAAGGGCGTTGCTATGGCAACGGGAGGCAGGGCCTGGTTGGGGGGACCGGGCCCGG
GCTGGGACCGGGGGGGCCGGCGGTGGCTGTGGTGGGGCGATGGCGGAGCGCGGCC
CGGCCTTCTGCGGCCTGTACGACACGTCCTCGCTGCTGCGATACTGCAACGATGACA
ATTTGTCGGGCACGAGCGGTATGGAAGTGGACGACCGCGTGTCGGCGCTGGAGCAG
CGGCTGCAGTTACAGGAAGACGAGCTGGCGGTCCTAAAGGCGGCGCTGGCGGATGC
TCTGCGTCGCCTGCGGGCATGCGAAGAACAGGGAGCGGCGCTACGCGCGCGGGGCA
CCCCCAAGGGCCGGGCGCCTCCGCGCTTAGGCACCACTGCCTCGGTGTGTCAGCTCT
TGAAAGGCCTTCCCACCAGGACGCCCCTTAATGGCTCGGGACCCCCGCGGCGCGTG
GGTGGCTATGCCACGTCCCCATCCTCTCCCAAGAAGGAGGCGACCTCCGGGCGCAG
CAGTGTCCGCCGCTACTTGTCACCAGAGCGCCTCGCCTCGGTGCGCCGTGAGGACCC
CCGCAGCCGGACCACATCCTCCAGCAGCAACTGTAGCGCCAAAAAGGAAGGCAAAA
CCAAAGAAGTTATCTTCAGTGTGGAGGATGGCTCCGTGAAAATGTTCCTGAGGGGCC
GCCCTGTGCCCATGATGATCCCAGACGAGCTGGCACCCACCTACAGCCTGGACACA
CGCTCGGAGCTGCCTTCTTGCCGGCTCAAGCTGGAGTGGGTCTATGGCTACCGTGGC
CGAGACTGCCGGGCCAACCTTTATTTGCTGCCCACCGGGGAGATAGTGTACTTTGTG
GCCTCCGTAGCCGTGCTATACAGCGTGGAGGAGCAGAGGCAGCGACACTACCTGGG
ACACAACGATGACATCAAATGCTTGGCCATCCACCCAGATATGGTCACCATCGCCAC
GGGACAGGTGGCGGGAACCACTAAGGAAGGGAAGCCGCTGCCGCCCCACGTGCGC
ATCTGGGACTCAGTTTCCCTCTCCACCTTACACGTGCTGGGCTTGGGGGTGTTTGACA
GAGCCGTGTGCTGTGTGGGCTTCTCCAAATCTAATGGAGGCAACCTGCTGTGTGCAG
TGGATGAATCCAATGATCACATGCTCTCGGTGTGGGACTGGGCCAAGGAGACCAAG
GTGGTGGATGTCAAGTGCTCCAATGAGGCTGTATTGGTGGCCACCTTCCACCCCACG
GACCCCACTGTGCTTATCACCTGCGGGAAATCTCACATCTACTTCTGGACCTTGGAG
GGGGGCAGCTTGAGCAAGCGGCAAGGCCTCTTTGAGAAACATGAGAAACCGAAGTA
TGTGCTGTGTGTGACCTTTTTGGAAGGTGGCGACGTGGTCACGGGGGACTCTGGGGG
GAACCTCTATGTTTGGGGCAAAGGTGGGAACCGTATCACACAGGCGGTGCTGGGCG
CCCACGACGGCGGCGTGTTTGGGCTCTGCGCCCTGCGGGACGGGACGCTGGTGTCTG
GAGGGGGCCGTGATCGGCGGGTGGTCCTCTGGGGTTCTGACTACAGCAAGCTGCAG
GAAGTGGAGGTCCCTGAGGACTTTGGCCCTGTGCGCACCGTGGCAGAGGGCCACGG
AGACACACTGTACGTGGGGACCACCCGCAATTCCATCCTGCAGGGCTCCGTGCACA
CAGGCTTCTCACTGCTGGTCCAGGGCCATGTGGAAGAGCTGTGGGGCCTGGCCACA
CACCCCAGTCGGGCCCAGTTTGTGACCTGCGGGCAGGATAAGCTGGTGCATCTATGG
AGCTCAGATTCCCACCAGCCCCTGTGGAGCAGGATCATCGAGGACCCTGCCCGCTCA
GCCGGCTTCCACCCCAGTGGCTCTGTCCTGGCTGTGGGTACAGTGACTGGCAGATGG
CTGCTGCTGGACACGGAGACCCATGACCTGGTGGCTATCCACACAGACGGCAATGA
ACAGATCTCAGTGGTCAGCTTCTCCCCAGACGGGGCGTACCTGGCCGTGGGCTCCCA
CGACAACTTGGTGTACGTGTACACGGTGGACCAGGGCGGCCGCAAGGTCAGCCGCC
TGGGCAAGTGCTCGGGCCATTCCAGTTTTATCACCCACCTGGATTGGGCCCAGGACA
GCAGCTGCTTTGTCACCAACTCCGGGGACTATGAGATTCTGTACTGGGACCCGGCTA
CCTGTAAGCAGATCACCAGTGCGGATGCTGTGAGGAACATGGAATGGGCCACAGCT
ACTTGTGTCCTAGGGTTTGGGGTGTTTGGGATCTGGTCTGAGGGGGCGGACGGCACT
GATATCAACGCTGTGGCCCGCTCTCATGATGGGAAGTTGCTGGCTTCAGCTGATGAC
TTTGGCAAAGTTCACCTGTTTAGCTACCCCTGCTGTCAGCCTCGAGCCCTCAGCCAC
AAGTACGGTGGACACAGCAGCCATGTGACAAATGTGGCCTTCTTGTGGGATGACAG
CATGGCCCTGACCACAGGGGGCAAGGACACCAGTGTGCTACAGTGGCGGGTGGTCT
GACTGCAGTCGACGGTACCGCGGGCCCGGGATCCACCGGATCTAGATAACTGATCA
TAATCAGCCATACCACATTTGTAGAGGTTTTACTTGCTTTAAAAAACCTCCCACACCT
CCCCCTGAACCTGAAACATAAAATGAATGCAATTGTTGTTGTTAACTTGTTTATTGC
AGCTTATAATGGTTACAAATAAAGCAATAGCATCACAAATTTCACAAATAAAGCATT
TTTTTCACTGCATTCTAGTTGTGGTTTGTCCAAACTCATCAATGTATCTTAACGCGTA
AATTGTAAGCGTTAATATTTTGTTAAAATTCGCGTTAAATTTTTGTTAAATCAGCTCA
TTTTTTAACCAATAGGCCGAAATCGGCAAAATCCCTTATAAATCAAAAGAATAGACC
GAGATAGGGTTGAGTGTTGTTCCAGTTTGGAACAAGAGTCCACTATTAAAGAACGTG
GACTCCAACGTCAAAGGGCGAAAAACCGTCTATCAGGGCGATGGCCCACTACGTGA
ACCATCACCCTAATCAAGTTTTTTGGGGTCGAGGTGCCGTAAAGCACTAAATCGGAA
CCCTAAAGGGAGCCCCCGATTTAGAGCTTGACGGGGAAAGCCGGCGAACGTGGCGA
GAAAGGAAGGGAAGAAAGCGAAAGGAGCGGGCGCTAGGGCGCTGGCAAGTGTAGC
GGTCACGCTGCGCGTAACCACCACACCCGCCGCGCTTAATGCGCCGCTACAGGGCG
CGTCAGGTGGCACTTTTCGGGGAAATGTGCGCGGAACCCCTATTTGTTTATTTTTCTA
AATACATTCAAATATGTATCCGCTCATGAGACAATAACCCTGATAAATGCTTCAATA
ATATTGAAAAAGGAAGAGTCCTGAGGCGGAAAGAACCAGCTGTGGAATGTGTGTCA
GTTAGGGTGTGGAAAGTCCCCAGGCTCCCCAGCAGGCAGAAGTATGCAAAGCATGC
ATCTCAATTAGTCAGCAACCAGGTGTGGAAAGTCCCCAGGCTCCCCAGCAGGCAGA
AGTATGCAAAGCATGCATCTCAATTAGTCAGCAACCATAGTCCCGCCCCTAACTCCG
CCCATCCCGCCCCTAACTCCGCCCAGTTCCGCCCATTCTCCGCCCCATGGCTGACTA
ATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAGATCGATCAAGAGACAGG
ATGAGGATCGTTTCGCATGATTGAACAAGATGGATTGCACGCAGGTTCTCCGGCCGC
TTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCG
ACCTGTCCGGTGCCCTGAATGAACTGCAAGACGAGGCAGCGCGGCTATCGTGGCTG
GCCACGACGGGCGTTCCTTGCGCAGCTGTGCTCGACGTTGTCACTGAAGCGGGAAG
GGACTGGCTGCTATTGGGCGAAGTGCCGGGGCAGGATCTCCTGTCATCTCACCTTGC
TCCTGCCGAGAAAGTATCCATCATGGCTGATGCAATGCGGCGGCTGCATACGCTTGA
TCCGGCTACCTGCCCATTCGACCACCAAGCGAAACATCGCATCGAGCGAGCACGTA
CTCGGATGGAAGCCGGTCTTGTCGATCAGGATGATCTGGACGAAGAGCATCAGGGG
CTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCG
CTTTTCTGGATTCATCGACTGTGGCCGGCTGGGTGTGGCGGACCGCTATCAGGACAT
AGCGTTGGCTACCCGTGATATTGCTGAAGAGCTTGGCGGCGAATGGGCTGACCGCTT
CCTCGTGCTTTACGGTATCGCCGCTCCCGATTCGCAGCGCATCGCCTTCTATCGCCTT
CTTGACGAGTTCTTCTGAGCGGGACTCTGGGGTTCGAAATGACCGACCAAGCGACGC
CCAACCTGCCATCACGAGATTTCGATTCCACCGCCGCCTTCTATGAAAGGTTGGGCT
TCGGAATCGTTTTCCGGGACGCCGGCTGGATGATCCTCCAGCGCGGGGATCTCATGC
TGGAGTTCTTCGCCCACCCTAGGGGGAGGCTAACTGAAACACGGAAGGAGACAATA
CCGGAAGGAACCCGCGCTATGACGGCAATAAAAAGACAGAATAAAACGCACGGTG
TTGGGTCGTTTGTTCATAAACGCGGGGTTCGGTCCCAGGGCTGGCACTCTGTCGATA
CCCCACCGAGACCCCATTGGGGCCAATACGCCCGCGTTTCTTCCTTTTCCCCACCCC
ACCCCCCAAGTTCGGGTGAAGGCCCAGGGCTCGCAGCCAACGTCGGGGCGGCAGGC
CCTGCCATAGCCTCAGGTTACTCATATATACTTTAGATTGATTTAAAACTTCATTTTT
AATTTAAAAGGATCTAGGTGAAGATCCTTTTTGATAATCTCATGACCAAAATCCCTT
AACGTGAGTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCGAAGGTAAC
TGGCTTCAGCAGAGCGCAGATACCAAATACTGTCCTTCTAGTGTAGCCGTAGTTAGG
CCACCACTTCAAGAACTCTGTAGCACCGCCTACATACCTCGCTCTGCTAATCCTGTT
ACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGA
GAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTT
TATAGTCCTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGGTTCCTG
GCCTTTTGCTGGCCTTTTGCTCACATGTTCTTTCCTGCGTTATCCCCTGATTCTGTGGA
TAACCGTATTACCGCCATGCAT
Variant 2 (EML2-L AAAA)
(SEQ ID NO: 6)
TAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCC
ATTGACGCAAATGGGCGGTAGGCGTGTACGGTGGGAGGTCTATATAAGCAGAGCTG
GTTTAGTGAACCGTCAGATCCGCTAGCGCTACCGGTCGCCACCATGGTGAGCAAGG
GCGAGGAGGATAACATGGCCTCTCTCCCAGCGACACATGAGTTACACATCTTTGGCT
CCATCAACGGTGTGGACTTTGACATGGTGGGTCAGGGCACCGGCAATCCAAATGAT
GGTTATGAGGAGTTAAACCTGAAGTCCACCAAGGGTGACCTCCAGTTCTCCCCCTGG
ATTCTGGTCCCTCATATCGGGTATGGCTTCCATCAGTACCTGCCCTACCCTGACGGG
ATGTCGCCTTTCCAGGCCGCCATGGTAGATGGCTCCGGcTACCAAGTCCATCGCACA
ATGCAGTTTGAAGATGGTGCCTCCCTTACTGTTAACTACCGCTACACCTACGAGGGA
AGCCACATCAAAGGAGAGGCCCAGGTGAAGGGGACTGGTTTCCCTGCTGACGGTCC
TGTGATGACCAACTCGCTGACCGCTGCGGACTGGTGCAGGTCGAAGAAGACTTACC
CCAACGACAAAACCATCATCAGTACCTTTAAGTGGAGTTACACCACTGGAAATGGC
AAGCGCTACCGGAGCACTGCGCGGACCACCTACACCTTTGCCAAGCCAATGGCGGC
TAACTATCTGAAGAACCAGCCGATGTACGTGTTCCGTAAGACGGAGCTCAAGCACTC
CAAGACCGAGCTCAACTTCAAGGAGTGGCAAAAGGCCTTTACCGATGTGATGGGCA
TGGACGAGCTGTACAAGTCCGGACTCAGATCTCGAGCTCAAGCTTCGATGCTGGAAC
GAAGGGCGTTGCTATGGCAACGGGAGGCAGGGCCTGGTTGGGGGGACCGGGCCCGG
GCTGGGACCGGGGGGGCCGGCGGTGGCTGTGGTGGGGCGATGGCGGAGCGCGGCC
CGGCCTTCTGCGGCCTGTACGACACGTCCTCGCTGCTGCGATACTGCAACGATGACA
ATTTGTCGGGCACGAGCGGTATGGAAGTGGACGACCGCGTGTCGGCGCTGGAGCAG
CGGCTGCAGTTACAGGAAGACGAGCTGGCGGTCCTAAAGGCGGCGgcGGCGGcTGCT
CTGCGTCGCCTGCGGGCATGCGAAGAACAGGGAGCGGCGCTACGCGCGCGGGGCAC
CCCCAAGGGCCGGGCGCCTCCGCGCTTAGGCACCACTGCCTCGGTGTGTCAGCTCTT
GAAAGGCCTTCCCACCAGGACGCCCCTTAATGGCTCGGGACCCCCGCGGCGCGTGG
GTGGCTATGCCACGTCCCCATCCTCTCCCAAGAAGGAGGCGACCTCCGGGCGCAGC
AGTGTCCGCCGCTACTTGTCACCAGAGCGCCTCGCCTCGGTGCGCCGTGAGGACCCC
CGCAGCCGGACCACATCCTCCAGCAGCAACTGTAGCGCCAAAAAGGAAGGCAAAAC
CAAAGAAGTTATCTTCAGTGTGGAGGATGGCTCCGTGAAAATGTTCCTGAGGGGCC
GCCCTGTGCCCATGATGATCCCAGACGAGCTGGCACCCACCTACAGCCTGGACACA
CGCTCGGAGCTGCCTTCTTGCCGGCTCAAGCTGGAGTGGGTCTATGGCTACCGTGGC
CGAGACTGCCGGGCCAACCTTTATTTGCTGCCCACCGGGGAGATAGTGTACTTTGTG
GCCTCCGTAGCCGTGCTATACAGCGTGGAGGAGCAGAGGCAGCGACACTACCTGGG
ACACAACGATGACATCAAATGCTTGGCCATCCACCCAGATATGGTCACCATCGCCAC
GGGACAGGTGGCGGGAACCACTAAGGAAGGGAAGCCGCTGCCGCCCCACGTGCGC
ATCTGGGACTCAGTTTCCCTCTCCACCTTACACGTGCTGGGCTTGGGGGTGTTTGACA
GAGCCGTGTGCTGTGTGGGCTTCTCCAAATCTAATGGAGGCAACCTGCTGTGTGCAG
TGGATGAATCCAATGATCACATGCTCTCGGTGTGGGACTGGGCCAAGGAGACCAAG
GTGGTGGATGTCAAGTGCTCCAATGAGGCTGTATTGGTGGCCACCTTCCACCCCACG
GACCCCACTGTGCTTATCACCTGCGGGAAATCTCACATCTACTTCTGGACCTTGGAG
GGGGGCAGCTTGAGCAAGCGGCAAGGCCTCTTTGAGAAACATGAGAAACCGAAGTA
TGTGCTGTGTGTGACCTTTTTGGAAGGTGGCGACGTGGTCACGGGGGACTCTGGGGG
GAACCTCTATGTTTGGGGCAAAGGTGGGAACCGTATCACACAGGCGGTGCTGGGCG
CCCACGACGGCGGCGTGTTTGGGCTCTGCGCCCTGCGGGACGGGACGCTGGTGTCTG
GAGGGGGCCGTGATCGGCGGGTGGTCCTCTGGGGTTCTGACTACAGCAAGCTGCAG
GAAGTGGAGGTCCCTGAGGACTTTGGCCCTGTGCGCACCGTGGCAGAGGGCCACGG
AGACACACTGTACGTGGGGACCACCCGCAATTCCATCCTGCAGGGCTCCGTGCACA
CAGGCTTCTCACTGCTGGTCCAGGGCCATGTGGAAGAGCTGTGGGGCCTGGCCACA
CACCCCAGTCGGGCCCAGTTTGTGACCTGCGGGCAGGATAAGCTGGTGCATCTATGG
AGCTCAGATTCCCACCAGCCCCTGTGGAGCAGGATCATCGAGGACCCTGCCCGCTCA
GCCGGCTTCCACCCCAGTGGCTCTGTCCTGGCTGTGGGTACAGTGACTGGCAGATGG
CTGCTGCTGGACACGGAGACCCATGACCTGGTGGCTATCCACACAGACGGCAATGA
ACAGATCTCAGTGGTCAGCTTCTCCCCAGACGGGGCGTACCTGGCCGTGGGCTCCCA
CGACAACTTGGTGTACGTGTACACGGTGGACCAGGGCGGCCGCAAGGTCAGCCGCC
TGGGCAAGTGCTCGGGCCATTCCAGTTTTATCACCCACCTGGATTGGGCCCAGGACA
GCAGCTGCTTTGTCACCAACTCCGGGGACTATGAGATTCTGTACTGGGACCCGGCTA
CCTGTAAGCAGATCACCAGTGCGGATGCTGTGAGGAACATGGAATGGGCCACAGCT
ACTTGTGTCCTAGGGTTTGGGGTGTTTGGGATCTGGTCTGAGGGGGCGGACGGCACT
GATATCAACGCTGTGGCCCGCTCTCATGATGGGAAGTTGCTGGCTTCAGCTGATGAC
TTTGGCAAAGTTCACCTGTTTAGCTACCCCTGCTGTCAGCCTCGAGCCCTCAGCCAC
AAGTACGGTGGACACAGCAGCCATGTGACAAATGTGGCCTTCTTGTGGGATGACAG
CATGGCCCTGACCACAGGGGGCAAGGACACCAGTGTGCTACAGTGGCGGGTGGTCT
GACTGCAGTCGACGGTACCGCGGGCCCGGGATCCACCGGATCTAGATAACTGATCA
TAATCAGCCATACCACATTTGTAGAGGTTTTACTTGCTTTAAAAAACCTCCCACACCT
CCCCCTGAACCTGAAACATAAAATGAATGCAATTGTTGTTGTTAACTTGTTTATTGC
AGCTTATAATGGTTACAAATAAAGCAATAGCATCACAAATTTCACAAATAAAGCATT
TTTTTCACTGCATTCTAGTTGTGGTTTGTCCAAACTCATCAATGTATCTTAACGCGTA
AATTGTAAGCGTTAATATTTTGTTAAAATTCGCGTTAAATTTTTGTTAAATCAGCTCA
TTTTTTAACCAATAGGCCGAAATCGGCAAAATCCCTTATAAATCAAAAGAATAGACC
GAGATAGGGTTGAGTGTTGTTCCAGTTTGGAACAAGAGTCCACTATTAAAGAACGTG
GACTCCAACGTCAAAGGGCGAAAAACCGTCTATCAGGGCGATGGCCCACTACGTGA
ACCATCACCCTAATCAAGTTTTTTGGGGTCGAGGTGCCGTAAAGCACTAAATCGGAA
CCCTAAAGGGAGCCCCCGATTTAGAGCTTGACGGGGAAAGCCGGCGAACGTGGCGA
GAAAGGAAGGGAAGAAAGCGAAAGGAGCGGGCGCTAGGGCGCTGGCAAGTGTAGC
GGTCACGCTGCGCGTAACCACCACACCCGCCGCGCTTAATGCGCCGCTACAGGGCG
CGTCAGGTGGCACTTTTCGGGGAAATGTGCGCGGAACCCCTATTTGTTTATTTTTCTA
AATACATTCAAATATGTATCCGCTCATGAGACAATAACCCTGATAAATGCTTCAATA
ATATTGAAAAAGGAAGAGTCCTGAGGCGGAAAGAACCAGCTGTGGAATGTGTGTCA
GTTAGGGTGTGGAAAGTCCCCAGGCTCCCCAGCAGGCAGAAGTATGCAAAGCATGC
ATCTCAATTAGTCAGCAACCAGGTGTGGAAAGTCCCCAGGCTCCCCAGCAGGCAGA
AGTATGCAAAGCATGCATCTCAATTAGTCAGCAACCATAGTCCCGCCCCTAACTCCG
CCCATCCCGCCCCTAACTCCGCCCAGTTCCGCCCATTCTCCGCCCCATGGCTGACTA
ATTTTTTTTATTTATGCAGAGGCCGAGGCCGCCTCGGCCTCTGAGCTATTCCAGAAGT
AGTGAGGAGGCTTTTTTGGAGGCCTAGGCTTTTGCAAAGATCGATCAAGAGACAGG
ATGAGGATCGTTTCGCATGATTGAACAAGATGGATTGCACGCAGGTTCTCCGGCCGC
TTGGGTGGAGAGGCTATTCGGCTATGACTGGGCACAACAGACAATCGGCTGCTCTG
ATGCCGCCGTGTTCCGGCTGTCAGCGCAGGGGCGCCCGGTTCTTTTTGTCAAGACCG
ACCTGTCCGGTGCCCTGAATGAACTGCAAGACGAGGCAGCGCGGCTATCGTGGCTG
GCCACGACGGGCGTTCCTTGCGCAGCTGTGCTCGACGTTGTCACTGAAGCGGGAAG
GGACTGGCTGCTATTGGGCGAAGTGCCGGGGCAGGATCTCCTGTCATCTCACCTTGC
TCCTGCCGAGAAAGTATCCATCATGGCTGATGCAATGCGGCGGCTGCATACGCTTGA
TCCGGCTACCTGCCCATTCGACCACCAAGCGAAACATCGCATCGAGCGAGCACGTA
CTCGGATGGAAGCCGGTCTTGTCGATCAGGATGATCTGGACGAAGAGCATCAGGGG
CTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCG
CTTTTCTGGATTCATCGACTGTGGCCGGCTGGGTGTGGCGGACCGCTATCAGGACAT
AGCGTTGGCTACCCGTGATATTGCTGAAGAGCTTGGCGGCGAATGGGCTGACCGCTT
CCTCGTGCTTTACGGTATCGCCGCTCCCGATTCGCAGCGCATCGCCTTCTATCGCCTT
CTTGACGAGTTCTTCTGAGCGGGACTCTGGGGTTCGAAATGACCGACCAAGCGACGC
CCAACCTGCCATCACGAGATTTCGATTCCACCGCCGCCTTCTATGAAAGGTTGGGCT
TCGGAATCGTTTTCCGGGACGCCGGCTGGATGATCCTCCAGCGCGGGGATCTCATGC
TGGAGTTCTTCGCCCACCCTAGGGGGAGGCTAACTGAAACACGGAAGGAGACAATA
CCGGAAGGAACCCGCGCTATGACGGCAATAAAAAGACAGAATAAAACGCACGGTG
TTGGGTCGTTTGTTCATAAACGCGGGGTTCGGTCCCAGGGCTGGCACTCTGTCGATA
CCCCACCGAGACCCCATTGGGGCCAATACGCCCGCGTTTCTTCCTTTTCCCCACCCC
ACCCCCCAAGTTCGGGTGAAGGCCCAGGGCTCGCAGCCAACGTCGGGGCGGCAGGC
CCTGCCATAGCCTCAGGTTACTCATATATACTTTAGATTGATTTAAAACTTCATTTTT
AATTTAAAAGGATCTAGGTGAAGATCCTTTTTGATAATCTCATGACCAAAATCCCTT
AACGTGAGTTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACCGCT
ACCAGCGGTGGTTTGTTTGCCGGATCAAGAGCTACCAACTCTTTTTCCGAAGGTAAC
TGGCTTCAGCAGAGCGCAGATACCAAATACTGTCCTTCTAGTGTAGCCGTAGTTAGG
CCACCACTTCAAGAACTCTGTAGCACCGCCTACATACCTCGCTCTGCTAATCCTGTT
ACCAGTGGCTGCTGCCAGTGGCGATAAGTCGTGTCTTACCGGGTTGGACTCAAGACG
ATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGA
GAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTT
TATAGTCCTGTCGGGTTTCGCCACCTCTGACTTGAGCGTCGATTTTTGTGATGCTCGT
CAGGGGGGCGGAGCCTATGGAAAAACGCCAGCAACGCGGCCTTTTTACGGTTCCTG
GCCTTTTGCTGGCCTTTTGCTCACATGTTCTTTCCTGCGTTATCCCCTGATTCTGTGGA
TAACCGTATTACCGCCATGCAT

### EXAMPLES

### Example 1: Materials and Methods

### Cell Culture

Cell lines (HeLa, CHL-1, COS7) were cultured in DMEM medium (Thermo Fisher Scientific, Cat #11965118) supplemented with 10% fetal bovine serum (FBS, Atlanta Biologicals, Cat #S11150) and penicillin-streptomycin (Thermo Fisher Scientific, Cat #15140122). Knock-in HeLa lines for EMLs 1, 2-L, 2-S, 3 and 4 and VASH1-SVBP (5) were cultured with 1 μg/ml puromycin (Sigma-Aldrich, Cat #P8833). Cells were maintained in the presence of 5% CO₂ at 37 °C. Expression of transgene(s) in knock-in cells was initiated by the addition of 2 μg/ml doxycycline (Thermo Fisher Scientific, Cat #BP26531). To evaluate the expression level and the size of the expressed proteins by immunoblot analysis, induced or transfected (for conditions, see below) cells were lysed with lysis buffer (6 mM Na₂HPO₄, 4 mM NaH₂PO₄, 2 mM EDTA, 150 mM NaCl, 1% NP40 and protease inhibitors) with a brief sonication followed by clarification.

### Plasmid Construction

Oligonucleotides and plasmids used in this study are summarized in Tables 1 and 2 (below), respectively. All PCR and mutagenesis were performed with Prime STAR Max DNA polymerase (Takara bio. Cat #R045B) Unless otherwise noted, Gibson assembly (NEB) was used to insert DNA fragments into vectors. cDNAs were obtained from Horizon Discovery: EML1 (Clone ID 5533599), EML2 (Clone ID 5177401; partial sequence), EML3 (Clone ID 3915493) and EML4 (Clone ID 9021713). A 36-nucleotide sequence corresponding to the PA tag (5′-GGCGTTGCCATGCCAGGTGCCGAAGATGATGTGGTG-3′ (SEQ ID NO:2)) was inserted right after the start codon of pEGFP-C1 and pEGFP-N1 resulting pPA-EGFP-C1 and pPA-EGFP-N1. EGFP of these vectors were modified to the monomeric EGFP with A207K mutation using an oligonucleotide TH460.

To clone EML2-L, EML2 cDNA was PCR-amplified with primers TH607 and TH608 and inserted into pPA-EGFP-C1 that had been linearized with EcoRI (pPA-EGFP-C1-EML2-partial). Since the inserted EML2 sequence lacks C-terminal 155-amino acid (aa) of EML2-L (isoform 3 (850 aa) on uniport database), a corresponding 465-nucleotide fragment was synthesized by gBlocks (Integrated DNA Technologies). gBlocks fragment was PCR amplified with primers TH610 and TH611 and inserted into EcoRI-treated pPA-EGFP-C1-EML2-partial. To match the database sequence of EML2 isoform 3, a single amino acid substitution of EML2L cDNA was corrected (L222V) using an oligonucleotide TH609. This vector, pPA-EGFP-C1-EML2-L, was further used as template to amplify EML2-S (uniport isoform 1 (649 aa)) using TH640 and TH611, and the amplified fragment was cloned into EcoRI-treated pPA-EGFP-C1 to assemble pPA-EGFPC1-EML2-S. These constructs were used as templates to make C-terminally-GFP-tagged EML2 constructs (pPA-EGFP-N1) with primers TH647 and TH648 (EML2-L) or TH649 and TH648 (EML2-S), or N-terminally-SNAP-tagged constructs (BamHI site of pSNAPf; NEB, Cat #N9183S) using primers TH657 and TH658 (for both EML2-L and S).

To generate pPA-EML2-L and S (N-terminal PA-tag), EGFP was removed from pPA-EGFPC1-EML2-L and S. pPA-EGFP-C1-EML2-L and S were PCR-amplified with phosphorylated primers TH661 and TH662 (EML2-L) or TH664 and TH665 (EML2-S) followed by ligation using T4 DNA ligase (NEB, Cat #M0202S). Site-directed mutagenesis was performed against pPAEML2-S for R69E, R69A/R341A (2RA). R69A/R314A/R316A/R341A (4RA) and L209R/Y254D (LR/YD) using primers TH748 (R69E), TH745 and TH746 (2RA), TH747 (additional 2 sites for 4RA) and TH750 and TH753 (LR/YD), respectively.

cDNAs of EML1, 3 and 4 were amplified using TH672 and TH702 (EML1), TH674 and TH675 (EML3) and TH676 and TH677 (EML4), and inserted into pPA-EGFP-C1 that had been PCR-amplified with TH670 and TH671 (EGFP was excluded) to generate pPA-EML1, 3 and 4 (N-terminal PA-tag). To make inducible HeLa cell lines for PA-tagged EMLs, corresponding pPAEML constructs were used as templates to generate PA-EML fragments with the following primers: TH641 and TH703 (PA-EML1), TH641 and TH642 (PA-EML2-L and S), TH641 and TH704 (PA-EML3) and TH641 and TH705 (PA-EML4) PA-EML fragments were then inserted into the pEM791 vector that had been digested with AgeI and BsrGI. The resulting pEM791-PAEML vectors were used to establish knock-in HeLa cell lines expressing PA-EMLs in a doxycycline inducible manner using recombination mediated cassette exchange (23).

To assemble pmNeonGreen-EML2-L, EML1, EML3 and EML4 (N-terminal mNeonGreentag), inserts were amplified with TH607 and TH611 (EML2-L), TH780 and TH781 (EML1), TH782 and TH783 (EML3) and TH784 and TH785 (EML4), and assembled into EcoRI-treated pmNeonGreen-C1. pmCherry-N1-EB3 was described previously (24).

pFastBac-EML2-S was assembled from an EML2-S fragment amplified with TH656 and TH655 and BamHI-digested pFastBac-HT A (Thermo Fisher Scientific). To introduce the R69E mutation, TH748 was used.

To generate GST-CTT(Y), GST-CTT(ΔY) constructs, a modified version of pGEX-KG vector where the thrombin site had been replaced with TEV protease recognition site, was used (pGEXKGT). The vector was PCR-amplified with phosphorylated primers TH511 and TH512 (Y) and TH511 and TH513 (ΔY) and ligated.

### Microtubule Co-Sedimentation Assay Using Lysates

Microtubule co-sedimentation was performed based on a published method (25). For the Y-conditions, WT HeLa cells were used For the ΔY conditions, VASH1-SVBP knock-in HeLa cells were cultured in the presence of 2 μg/ml doxycycline for 4 days. Cells were harvested with trypsin, rinsed in DPBS (Thermo Fisher Scientific, Cat #14190144) twice and suspended in ice-cold BRB80 (80 mM PIPES, 1 mM EGTA, 1 mM MgCl₂ pH 6.8) supplemented with protease inhibitor cocktail (Complete mini EDTA free; Roche, Cat #04693159001) and 1 mM DTT (Sigma-Aldrich, Cat #D9779). After sonication for 10 sec×4 times, lysates were cleared by centrifugation at 100,000×g for 1 hour at 4 °C. To induce microtubule assembly, 10 μM Taxol (Sigma-Aldrich, Cat #T7191) and 1 mM GTP (Sigma-Aldrich, Cat #G8877) were added and incubated at 37 °C for 25 min. The assembly mix was layered on a pre-warmed sucrose cushion (5% sucrose in BRB80 with 10 μM Taxol and 1 mM GTP) and centrifuged at 80,000×g for 30 min at 37 °C. Microtubule pellets were rinsed with BRB80-Taxol/GTP twice, resuspended in BRB80 supplemented with 8 M urea and incubated on ice for 10 min. Protein concentration was measured by Bradford Protein Assay (Bio-Rad, Cat #5000006). The assay was repeated three times on different days and a total of 6 samples (each 25 μg of tubulin+MAPs) were subjected to a TMT analysis. For CPA-mediated tubulin detyrosination in the WT HeLa lysate, 1/400 the lysate volume of CPA (Sigma-Aldrich, Cat #C9268) was added to the assembly mix and incubated on ice for 3 min prior to the microtubule assembly incubation.

### GST-CTT Pulldown

pGEX-KGT-αCTT constructs were transformed into BL21 (DE3). IPTG-induced gene expression occurred for 4 h at 25 °C. Bacteria were pelleted, rinsed in PBS once and resuspended in lysis buffer (PBS, 1 mM MgCl₂, 0.5% Triton-X100, 1 mM ATP, 1 mM DTT, 1 mM PMSF, 0.1 mg/ml lysozyme (Sigma-Aldrich, Cat #L6876) and Benzonase nuclease (Sigma-Aldrich, Cat #E1014). Bacterial lysates were sonicated and centrifuged (100,000×g, 30 min, 4 °C). Cleared lysates were loaded onto Glutathione sepharose 4B columns (GE Healthcare, Cat #17075601) that had been pre-equilibrated with the lysis buffer. Columns were washed with 10 column volumes each of wash buffer 1 (PBS, 1 mM MgCl₂, 1 mM ATP, 1 mM DTT, and 0.2 mM PMSF) and wash buffer 2 (PBS, 1 mM DTT, and 0.2 mM PMSF), and GST-CTT proteins were eluted with elution buffer (PBS with 10 mM reduced glutathione) Glutathione was removed by repeated ultrafiltration using Amicon Ultra-4 10K (Merck Millipore, Cat #UFC801024D) and protein concentration in the final fractions were measured with Bradford protein assay.

The pulldown assay was performed as follows. Glutathione Sepharose 4B resin (24 μl for each reaction) was rinsed in PBS 5 times prior to the addition of 370 μg of GST, GST-CTT (Y and ΔY). GST-resin conjugates were incubated on ice for 1 h and excess proteins were removed by 3 times of wash with the pulldown buffer (50 mM Tri-HCl, pH 8.0, 50 mM NaCl). CHL-1 cell lysates were prepared in lysis buffer (pulldown buffer supplemented with protease inhibitor cocktail (Complete mini EDTA free), 0.2 mM PMSF and 1 mM DTT). After sonication, lysates were cleared by centrifugation (56,000×g, 20 min, 4 °C). Lysates (corresponding to cells grown on two 15-cm dishes) were added to each GST-resin conjugate and gently mixed for 30 min at 4 °C. For the negative control, lysates were replaced with lysis buffer Resins were washed with the lysis buffer twice followed by the pulldown buffer three times. Bound proteins were eluted with GST proteins with 60 mM reduced glutathione in the pulldown buffer and analyzed on a precast gel.

For the identification of bound proteins with MS analysis, the above elution step was modified to avoid introducing overwhelming amounts of GST and GST-CTT proteins to the MS. After rinsing the resins with the pulldown buffer, resins were resuspended in pulldown buffer supplemented with 1 mM EDTA and 1 mM DTT. His-tagged TEV protease was added and incubated overnight at 4 °C. After quick spin, the supernatants were transferred to new tubes and mixed with Ni-NTA resins that had been equilibrated with the pulldown buffer. After 2 h of incubation at 4 °C, supernatants were recovered and subjected to the MS analysis.

### Mass Spectrometry

For the 6 samples generated in the microtubule co-sedimentation assay, each 25 μg protein sample was digested and labeled with TMT 6-plex isobaric tags following the manufacturer's protocol (Thermo Fisher). Samples were first diluted to ~1 M urea, reduced with 5 mM DTT for 30 min at 45 °C, then alkylated with 15 mM 2-chloracetamide for 30 min at room temperature in the dark. Sequencing grade modified trypsin (Promega) was added at a 1:25 enzyme:protein for overnight (~16 hr) digestion at 37 °C with constant mixing. Digestion was stopped by acidification, and peptides desalted using SepPak C18 cartridges according to the manufacturer's protocol (Waters, Cat #WAT023501). Samples were completely dried using a vacufuge and reconstituted in 100 μl of 0.1M TEAB. TMT 6-plex reagents were dissolved in 41 μl anhydrous acetonitrile, to which each digest was added and incubated at room temperature for 1 h. Reactions were quenched by adding 8 μl of 5% hydroxylamine for 15 min. Labeled samples were mixed together and dried via vacufuge. Offline high pH reversed-phase fractionation of the combined sample into 8 fractions was performed according to the manufacturer's protocol (Thermo Fisher, Cat #84868). Fractions were dried and reconstituted in 9 μl of 0.1% formic acid/2% acetonitrile in preparation for LCMS/MS analysis. For each fraction, a 2 μl aliquot was analyzed by LC-MS/MS using a RSLC Ultimate 3000 nano-UPLC (Dionex) with a 50 cm, 75 μm i.d. C18 column (Thermo Fisher Cat #ES903) and an Orbitrap Fusion (Thermo Fisher). A 3-hour gradient at 300 nl/min using 0.1% formic acid/acetonitrile (2-22% acetonitrile in 150 min; 22-32% acetonitrile in 40 min; 20 min wash at 90% followed by 50 min re-equilibration) was used, and eluent was introduced in the mass spectrometer via an EasySpray source (Thermo Fisher). The mass spectrometer was set to collect MS1 scans (Orbitrap; 120K resolution; AGC target 2×105; max IT 100 ms) followed by Top Speed MS2 scans (0.5 m/z isolation width, collision induced dissociation; ion trap; NCE 35; AGC 5×103; max IT 100 ms). For multinotch-MS3, the top 10 precursors from each MS2 were fragmented by HCD followed by Orbitrap analysis (NCE 55; 60K resolution; AGC 5×104; max IT 120 ms, 100-500 m/z scan range). Proteome Discoverer (v2.3; Thermo Fisher) was used for data analysis. Spectral files were searched against the SwissProt human protein database (20353 protein sequences downloaded Jun. 20, 2019) using the following search parameters: MS1 and MS2 tolerance were set to 10 ppm and 0.6 Da, respectively; carbamidomethylation of cysteines and TMT labeling of lysine and N-termini of peptides were considered static modifications; oxidation of methionine and deamidation of asparagine and glutamine were considered variable. Identified proteins and peptides were filtered to retain only those passing a 1% FDR threshold. Quantification was performed using high-quality MS3 spectra (average signal-to-noise ratio>6 and <30% isolation interference). For the analysis of EML2 isoforms, a separate search was conducted using the same SwissProt human database with EML2 isoforms 1, 2, and 3 added (UniProt identifiers O95834-1, O95834-2, and O95834-3). Abundances of peptides unique to isoforms 2 and 3 were compared between VASH and WT samples.

For the supernatants from the GST-CTT pulldown, cysteines were reduced then alkylated with 10 mM DTT at 45 °C for 30 min and 65 mM 2-chloroacetamide for 30 min at room temperature in the dark, respectively. Sequencing grade modified trypsin (Promega) was added at a 1:50 enzyme:protein for overnight digestion at 37 °C with constant mixing. Digestion was stopped by acidification, and peptides desalted using SepPak C18 cartridges according to the manufacturer's protocol (Waters, Cat #WAT023501). Eluate was dried using vacufuge, then peptides were reconstituted in 8 μl of 0.1% formic acid/2% acetonitrile solution in preparation for LC-MS/MS analysis. For each sample, 2 μl of the peptide solution were resolved on a 50 cm, 75 μm i.d. C18 column (Thermo Fisher, Cat #ES903) using a 0.1% formic acid/2% acetonitrile (Buffer A) and 0.1% formic acid/95% acetonitrile (Buffer B) gradient at 300 nl/min over a period of 180 min (2-22% buffer B in 110 min, 22-40% in 25 min, 40-90% in 5 min followed by holding at 90% buffer B for 5 min and requilibration with Buffer A for 25 min). Eluent was directly introduced into Orbitrap Fusion tribrid mass spectrometer (Thermo Fisher) using an EasySpray source. MS1 scans were acquired at 120K resolution (AGC target 2×105; max IT 100 ms). HCD MS/MS spectra were acquired using the Top speed method following each MS1 scan (NCE ~32%; AGC target 5×104; max IT 50 ms, 15K resolution) with 0.8 m/z isolation width. Spectra were searched against a SwissProt human database (28476 reviewed entries; downloaded on Aug. 29, 2018) appended with GST-CTT-Tyr protein using Proteome Discoverer (v2.1, Thermo Fisher). Search parameters included MS1 mass tolerance of 10 ppm and fragment tolerance of 0.2 Da; two missed cleavages were allowed; carbamidomethylation of cysteine was set as a fixed modification, and oxidation of methionine, deamidation of asparagine and glutamine were set as variable modifications. False discovery rates (FDRs) were estimated with Percolator, and peptides and proteins were filtered to 1% FDR.

### Immunofluorescence Staining

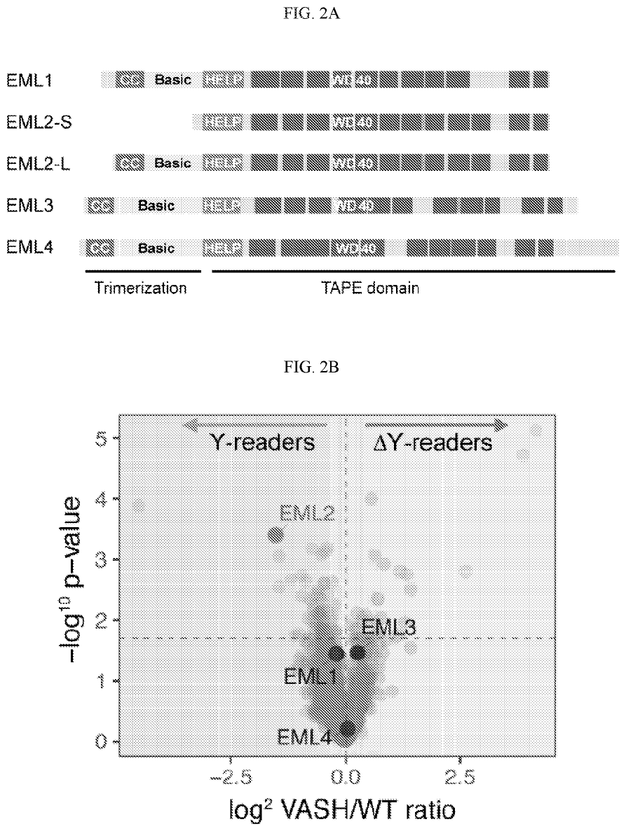

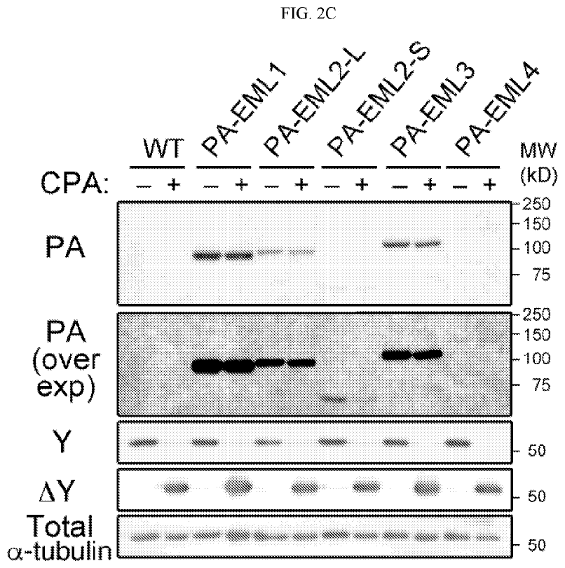

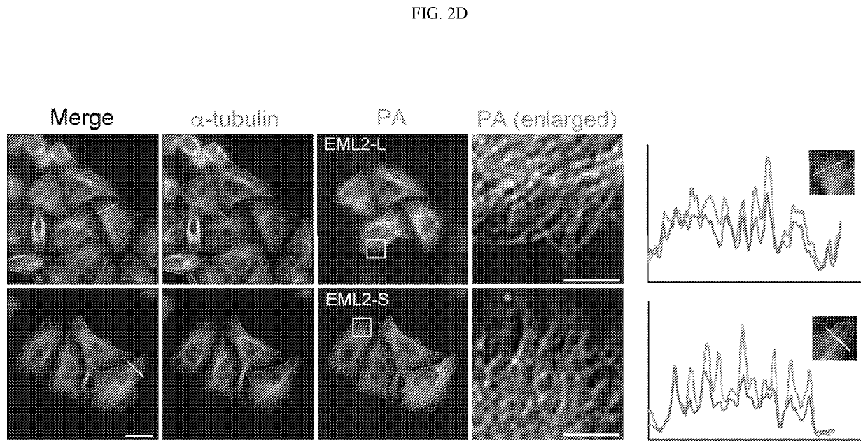

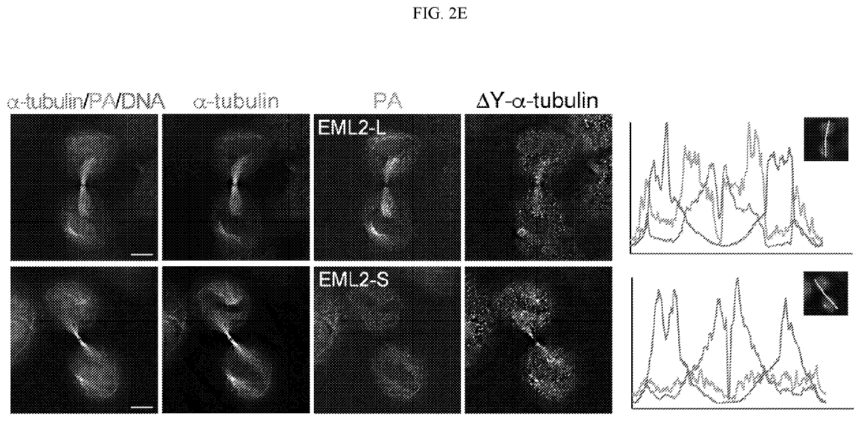

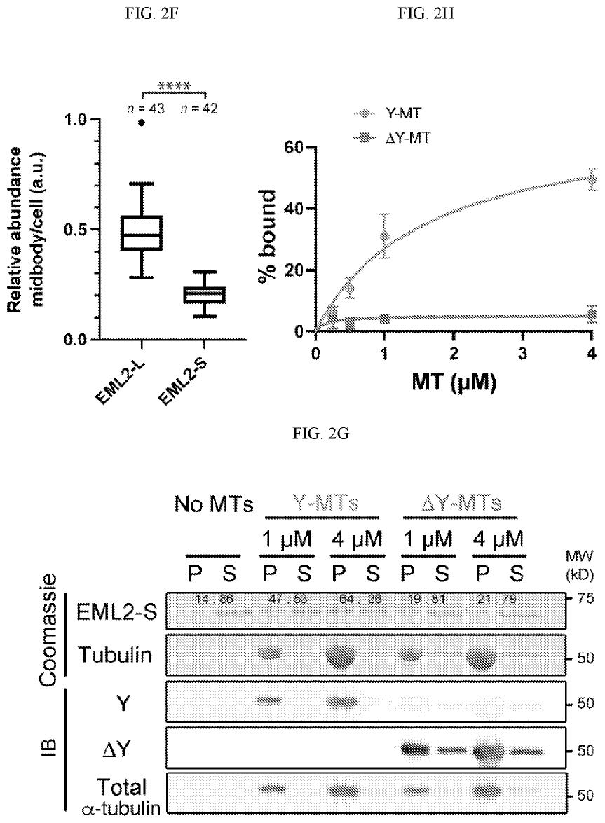

HeLa cells were seeded on coverslips 24 h prior to transfection using lipofectamine 2000 (Invitrogen, Cat #11668030). Each 1 g of plasmid was used per well on a 6-well plate. After 3 h of incubation with plasmid-transfection reagent in Opti-MEM (Thermo Fisher Scientific, Cat #31985-070), cells were rinsed and cultured in DMEM containing FBS and antibiotics overnight and subjected to staining. For nocodazole treatment, coverslips were soaked in culture media containing 664 nM nocodazole (Sigma-Aldrich, Cat #M1404) for 16 sec prior to the fixation Staining was performed as described (5). Antibodies were applied in the following order: Anti-PA tag antibody (clone NZ-1, Fujifilm Wako Pure Chemical, Cat #016-25861, diluted at 1:500, 45 min), anti-rat Alexa Fluor 594 (Thermo Fisher, Cat #A11007, diluted at 1:2,000, 35 min) and anti α-tubulin DM1α conjugated with FITC (Sigma-Aldrich, Cat #F2168, diluted at 1:500, 30 min). When DY-tubulin was co-stained, anti-detyrosinated α-tubulin antibody (Clone RM444; RevMAb Biosciences, Cat #31-1335-00, final concentration 1 μg/ml, 45 min) and then anti-rabbit Alexa Fluor 647 (Thermo Fisher, Cat #A21245, diluted at 1:2,000, 35 min) were applied between Rat Alexa Fluor 594 and DM1α-FITC staining. Images were obtained with a DeltaVision microscope equipped with an Olympus Plan Apo N 60×/1.42 oil immersion lens and deconvolved. Images of single optical sections were presented. Line profile was analyzed with Fiji/ImageJ (line width, 5 pixels across interphase microtubules in FIG. 2D; 15 pixels along midbody microtubules in FIG. 2E). To quantitatively evaluate the accumulation of PA-EML2-L or PA-EML2-S in the midbody microtubules, first, the cell boundary was manually traced, and midbody regions were determined by the 0.5 percentile thresholding of the blurred microtubule images with Gaussian Filter (Sigma=6 pixels). Then the PA intensities were measured and normalized against α-tubulin intensities in both regions. The relative PA intensity in the midbody divided by the relative PA intensity in the cell was calculated as an indicator of the accumulation of PA-EML2 in the midbody. For colocalization analysis between microtubules and PA-EML2-S, first, the cell boundary was manually defined, then fluorescent signals inside the cells were measured and averaged to confirm that the expression level of EML2-S and microtubule density do not differ among mutant constructs tested. The colocalization indicator, threshold overlap score (TOS), with the top 25 percentile was analyzed using the ImageJ plugin EzColocalization (26). Statistical analyses were performed with GraphPad Prism 9 using Wilcoxon rank sum exact test. Data were obtained for numbers indicated in each graph from at least three independent experiments.

### Live Cell Imaging

HeLa cells were plated on glass bottom dishes coated with poly-L-lysine (MatTek, Cat #P35GC-1.5-14-C) 24 h prior to transfection. Each 0.1-1 μg of plasmid per dish was used for transfection with Lipofectamine 2000. Cells were subject to imaging the next day. SNAP-tag was labeled with a SNAP-tag substrate (SNAP-Cell Oregon Green, NEB, Cat #S9104S) according to the manufacturer's instructions. Prior to imaging, culture medium was switched to L-15 medium (Thermo Fisher Scientific, Cat #21083027) containing 10% FBS, penicillin-streptomycin. Images were captured at 37 °C using a DeltaVision Elite microscope equipped with an 60× objective, a 1.59× magnifier, a Photometrics Prime 95B sCMOS camera, and an environmental chamber (GE Healthcare). Time lapse images for NeonGreen-tagged EMLs and EB3-mCherry were acquired at each 50 msec exposure every 1 sec for a total of 5-15 minutes. Deconvolved single plane images were used for the kymograph analysis using Multi Kymograph plug-in of Fiji/ImageJ and generation of movies.

### Immunoblot Analysis

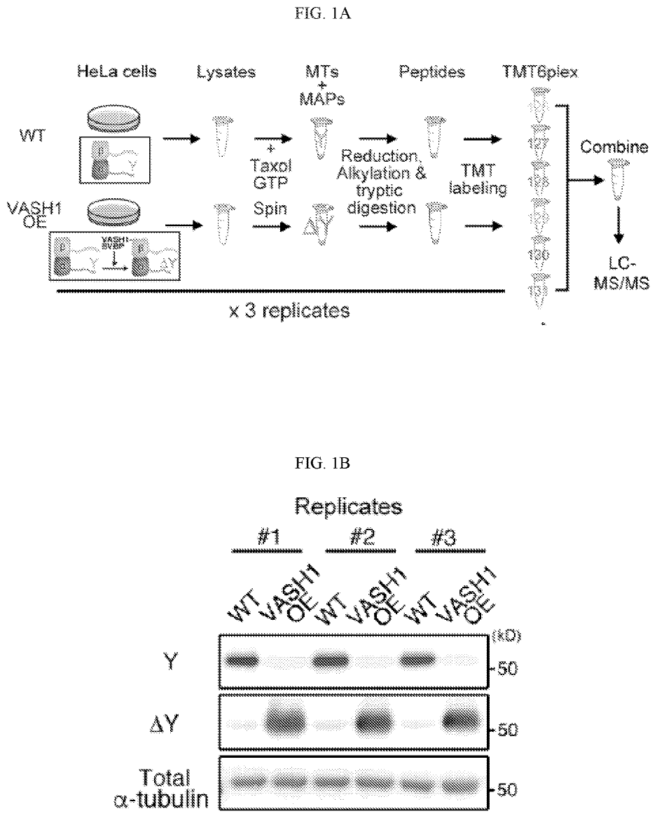

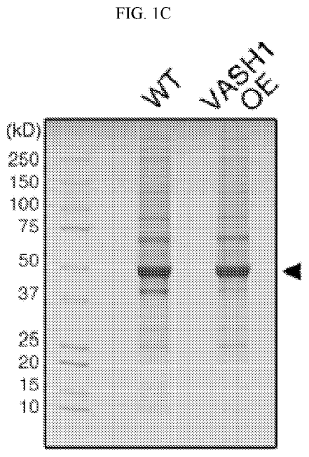

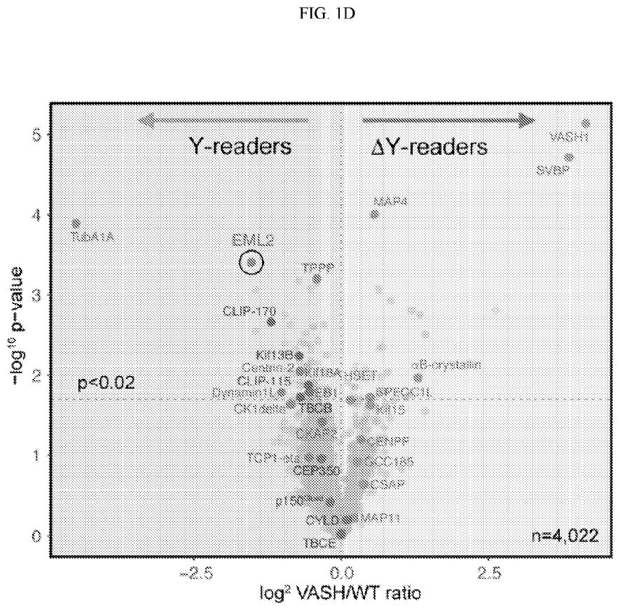

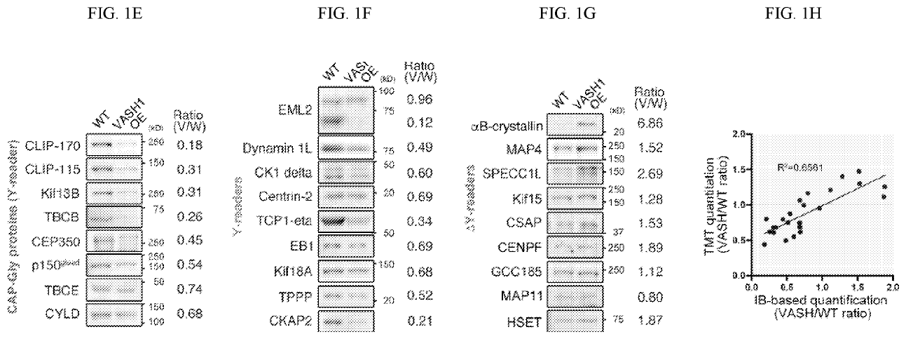

Proteins were separated on 10% acrylamide gels except for gels in FIGS. 1C and S3C being done with precast gradient gels (Thermo Fisher, Cat #XP04205BOX). Proteins were transferred onto nitrocellulose membranes and briefly stained with Ponceau S (Sigma-Aldrich, Cat #P7170). Blocking was performed with 5% skim milk solution in PBS supplemented with 0.05% tween 20 (PBST) for 1 hour at room temperature. Primary antibody reaction was performed at 4 °C overnight. After washing with PBST for 10 min×3 times, secondary antibodies were applied and incubated for 1 hour at room temperature. After another three times of wash, membranes were imaged with Azure c600 imager (Azure Biosystems). Antibodies used are listed in Table S3. Quantification was performed with the gel analyzer function of Fiji/ImageJ. First, band intensities of each reader in the microtubule pellet fractions (WT and VASH1 OE) were measured and normalized against corresponding total α-tubulin (DM1α), and then relative abundance was calculated (VASH1 OE over WT). To compare the IB- and TMT-based quantification methods (FIG. 1H), a TMT score averaged from three independent preparations were compared with a relative abundance calculated for a representative blot for each reader candidate shown in FIG. S2 (except for αB-crystallin and SPECC1L).

### Purification of Tubulin for TIRF Microscopy Assays

Tubulin was purified from bovine brains as previously described (27) with the modification of using Fractogel EMD SO3-(M) resin (Millipore-Sigma) instead of phosphocellulose. Tubulin was labeled using CF640R NHS-Ester (Biotium) and tetramethylrhodamine (TAMRA, Invitrogen) as described (28). An additional cycle of polymerization/depolymerization was performed before use. Protein concentrations were determined using a DS-11 FX spectrophotometer (DeNovix, Inc.).

Purification of Tubulin from HeLa Cells

Tubulin was purified from HeLa Kyoto cells using TOG affinity chromatography as described previously (5, 29, 30). To obtain detyrosinated HeLa tubulin, cleared HeLa lysate was supplemented with CPA (1/100 the cell pellet volume) and incubated on ice for 20 min before being loaded on a TOG column. Concentration of tubulin was calculated by measuring A280 on NanoDrop (Thermo Fisher) and using an extinction coefficient of tubulin (115,000 M⁻¹cm⁻¹).

Purification of EML2-S Protein from Insect Cells

Production of recombinant His-EML2-S protein was performed based on the Bac-to-bac Baculovirus expression system (Thermo Fisher). Baculoviral amplification was carried out in Sf9 cells cultured in Grace's insect medium (Thermo Fisher, Cat #11605-094) supplemented with 10%/FBS, 0.1% Pluronic F-68 (Thermo Fisher, Cat #24040-032) and antibiotics. P3 virus was then infected to High Five cells maintained in Insect-XPRESS medium (Lonza, Cat #12-730Q) with antibiotics. Three days after the addition of P3 virus, cells were harvested and stored at −80 °C until use.

Protein was first purified with Ni-NTA affinity chromatography and optionally further subjected to the polishing step with size exclusion chromatography. Insect cells were thawed, suspended in lysis buffer (PNI buffer (50 mM Sodium phosphate pH 7.4, 500 mM NaCl, 20 mM imidazole) supplemented with 0.2 mM PMSF (Sigma-Aldrich, Cat #78830), and protease inhibitor cocktail (SIGMAFAST, Sigma-Aldrich, Cat #S8820)) and sonicated. After centrifugation at 18,5000×g for 20 min at 4 °C), lysate was mixed with Ni-NTA agarose resin that had been preequilibrated with lysis buffer and incubated for 1 hour at 4 °C with gentle agitation. Agarose resin was washed in wash buffer 1 (PNI supplemented with 1 mM MgCl₂, 1 mM ATP and protease inhibitor) and wash buffer 2 (PNI with protease inhibitor) three times for each. Protein was eluted by PNI buffer whose imidazole concentration was elevated to 200 mM. For His-EML2-S proteins intended to the use in the in vitro microtubule co-sedimentation assay, imidazole was removed by PD-10 desalting columns (GE Healthcare, Cat #17085101) and protein was concentrated with Amicon Ultra-4 10K (Merck Millipore, Cat #UFC801024D). For further polishing of the protein for the in vitro dynamics assay, Ni-NTA eluate was loaded onto HiLoad Superdex 200 size exclusion column equilibrated with 10 mM Hepes pH 7.5, 300 mM KCl and 1 mM DTT. A peak fraction corresponding to the expected molecular weight of His-EML2-S (73.8 kD) was pooled and concentrated. Protein concentration was measured by Bradford protein assay using BSA as a standard. Purified protein was aliquoted, snap frozen and stored at −80 °C until use.

### Microtubule Co-Pelleting Assay Using Purified Proteins

HeLa tubulin (Y and ΔY) and His-EML2-S were thawed and centrifuged at 100,000×g for 10 min at 4 °C to remove any protein aggregates. Microtubule assembly was initiated by adding 1 mM GTP to cleared tubulin solution (5-10 mg/ml) and incubated at 37 °C for 15 min. Taxol was added to the reaction at final concentrations of 0.1, 1.0 and 10 μM successively with 10 min intervals. After 15 min of final incubation, microtubules were pelleted by centrifugation at 25,000×g at 25 °C for 15 min and resuspended in BRB80 supplemented with 10 M Taxol (BRB80-Taxol). To measure microtubule concentration, a small volume of microtubule solution was diluted into icecold BRB80 supplemented with 50 mM KCl and 1M CaCl₂, and incubated on ice to completely disassemble microtubules. Tubulin concentration was calculated from the A280 measurements and the extinction coefficient of tubulin (115,000 M₋₁cm₋₁). Binding of EML2-S to microtubules was carried out by incubating 500 nM His-EML2-S protein and 0.25, 0.5, 1.0 and 4 μM of Y- or ΔY microtubules in BRB80-Taxol for 20 min at room temperature. After centrifugation at 25,000×g at 25 °C for 20 min, supernatants and pellets were separated, and SDS-PAGE sample buffer was added to each fraction. For each reaction, a total of 814 ng or 136 ng of His-EML2-S protein (pellet+supernatant) were separated on SDS-PAGE gels for Coomassie staining or immunoblot analysis, respectively. Gels were stained with colloidal Coomassie, destained with water and scanned with a flatbed scanner (Cannon). Blots were images with Azure c600 (Azure Biosystems). Densitometric analysis was performed using the gel analyzer function of Fiji/ImageJ. Data analysis and non-linear fitting were performed on GraphPad Prism 9.

### Homology Modeling and Protein Electrostatics

EML2-S and EML1 have 69% sequence identity and 91% sequence homology, with no insertions or deletions in the β-propeller domains. This high degree of homology makes structural modeling relatively simple and using the x-ray structure of EML1 (pdb 4ic8) (12) we created a homology using basic threading of the EML2-S sequence into the EML1 structure. Missing atoms and hydrogens were added using VMD (31). The electrostatic potential was calculated using APBS (32). Calculations were performed at 50 mM NaCl using the linearized Poisson-Boltzmann equation, single Debye-Huckel boundary conditions, a protein dielectric of 2.0, and CHARMM partial charges (33). The potential was saved with ~-0.5 A grid spacing and visualized using VMD.

In Vitro Microtubule Dynamics Assay Using Purified his-EML2-S Protein

To visualize dynamic microtubules, we reconstituted microtubule growth off of GMPCPP double stabilized microtubule ‘seeds’ (17). Cover glass was cleaned in acetone, sonicated in 50% methanol, sonicated in 0.5 M KOH, exposed to air plasma (Plasma Etch) for 3 min, then silanized by soaking in 0.2% Dichlorodimethylsilane (DDS) in n-Heptane for 2 hours. 5 μl flow channels were constructed using two pieces of silanized cover glasses (22×22 mm and 18×18 mm) held together with double-sided tape and mounted into custom-machined cover slip holders. GMPCPP seeds were prepared by polymerizing a 1:4 molar ratio of TAMRA labeled:unlabeled tubulin in the presence of guanosine-5′-[(α, β)-methyleno]triphosphate (GMPCPP, Jena Biosciences) in two cycles, as described (17). Channels were first incubated with anti-TAMRA antibodies (Invitrogen) and then blocked with 5% Pluronic F-127. Flow channels were washed 3× with BRB80 before incubating with GMPCPP seeds. On each day of experiments tubes of unlabeled and CF642R labelled tubulin was thawed and mixed at a 1:17 molar ratio and then sub-aliquoted and refrozen in liquid nitrogen. For consistency in microtubule growth dynamics, one sub-aliquot of tubulin was used for each experiment. Microtubule growth from GMPCPP seeds was achieved by incubating flow channels with tubulin in imaging buffer: BRB80, 1 mM GTP, 0.1 mg/mL BSA, 0.01% Methylcellulose, 10 mM dithiothreitol, 250 nM glucose oxidase, 64 nM catalase, and 40 mM D-glucose, with the addition of purified His-EML2-S.

Data was acquired with a customized Zeiss Axio Observer 7 equipped with a Laser TIRF III, 405/488/561/638 nm lasers, and an Alpha Plan-Apo 100×/1.46 Oil DIC M27 Objective with objective heater 25.5/33 S1 set to 35 °C. Images were recorded on a Prime 95B CMOS camera (Photometrics) with a pixel size of 110 nm. Image acquisition was controlled using ZEN 2.3 (Zeiss). Images were acquired at 3 sec intervals.

All images were processed and analyzed using Fiji/ImageJ. If needed, prior to analysis images were corrected for stage drift using a drift correct script (Hadim). Microtubule dynamics were analyzed using kymographs. Growth and shrinkage rates were measured by manually drawing lines on kymographs and measuring the slope of growth or shrinkage. Catastrophe frequency was calculated by counting the total number of catastrophe events over the total time of all microtubule growth within a channel. Rescue frequency was calculated by counting the number of rescue events per total time or distance of microtubule disassembly.

All functions were fitted and graphed with OriginPro2020 (OriginLab) or Python 3 (available at python.org) using a JupyterLab Notebook. Mean and standard deviation were calculated using Excel. Images were linearly adjusted in brightness and contrast using Photoshop (Adobe). All final figures were assembled using Illustrator (Adobe).

### In Vitro Microtubule Dynamics Assay Using Cell Lysates

To prepare extracts for microtubule dynamics assays, COS-7 cells 16 h post-transfection were harvested and centrifuged at low-speed at 4 °C. The cell pellet was washed with PBS and resuspended in ice-cold BRB80 buffer freshly supplemented with 1 mM ATP, 1 mM PMSF and protease inhibitors cocktail. The cells were lysed by sonication (Fisher Scientific, Sonic Dismembrator Model 500) using 10% power, 4×10 sec on ice. After centrifugation for 10 min at 20,000 g at 4 °C, aliquots of the supernatant were snap frozen in liquid nitrogen and stored at −80 °C until further use.

The concentration of mNG-EML2-L in the COS-7 lysates was measured by a dot-blot, in which the same volumes of COS-7 lysates and a series of diluted known concentration of His-EML2-S protein were spotted onto a nitrocellulose membrane that was air-dried for 1 h and immunoblotted with a primary antibody to EML2 (Proteintech, Cat #13529-1-AP) overnight at 4 °C and secondary antibody 680 nm-anti rabbit (Jackson ImmunoResearch Laboratories Inc.) at room temperature for 1 h. The fluorescence intensity of the spots on the nitrocellulose membrane was detected by Azure c600 and quantified based on the standard curve of known concentration of EML2 protein using Fiji/ImageJ (NIH).

For the microtubule dynamics assay, a flow cell (~10 μl volume) was assembled by attaching a clean #1.5 coverslip (Fisher Scientific) to a glass slide (Fisher Scientific) with two strips of double-sided tape. Microtubule seeds containing 10% biotin-labeled tubulin and 10% Xrhodamine-labeled (Cytoskeleton Inc.) were generated by polymerization in the presence of GMPCPP (Jena Bioscience, Cat #NU-405S) and then immobilized on the coverslip by incubating the flow chamber sequentially with the following solutions: (1) 1 mg/ml BSA-biotin (Sigma. Cat #A8549), (2) blocking buffer (1 mg/ml BSA in BRB80), (3) 0.5 mg/ml NeutrAvidin (ThermoFisher, Cat #31000), (4) blocking buffer, (5) GMPCPP-stabilized microtubule seeds, (6) blocking buffer. Microtubule growth was then initiated by flowing in 10 μM tubulin containing 12.5% Hilyte647-labeled tubulin (Cytoskeleton Inc.) together with BRB80 buffer (control) or cell lysate expressing mNG-EML2-L protein in reaction buffer [BRB80 supplemented with 1 mM GTP, 3 mM MgCl₂, 1 mM DTT, 1 mg/ml casein, 0.1% methylcellulose (Sigma) and oxygen-scavenging system (12.5 mM glucose, 0.05 mg/ml catalase, 0.25 mg/ml glucose oxidase)]. The flow cells were sealed with molten paraffin wax and imaged by TIRF microscopy. The temperature was set at 35 °C in a temperature-controlled chamber (Tokai Hit). Time-lapse images were acquired in 488 nm, 561 nm and 640 nm channels at a rate of every 2 s for 5 min. To determine the shrinking rate of microtubule plus ends, maximum intensity projections were generated and kymographs (width=3 pixels) were prepared using Fiji/ImageJ2 and displayed with time on the y axis and distance on the x axis. Only microtubule shrinking event with a slope over a three-pixel length were analyzed.

### Example 2

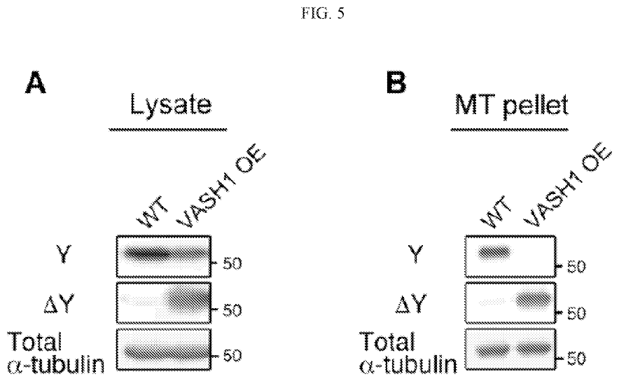

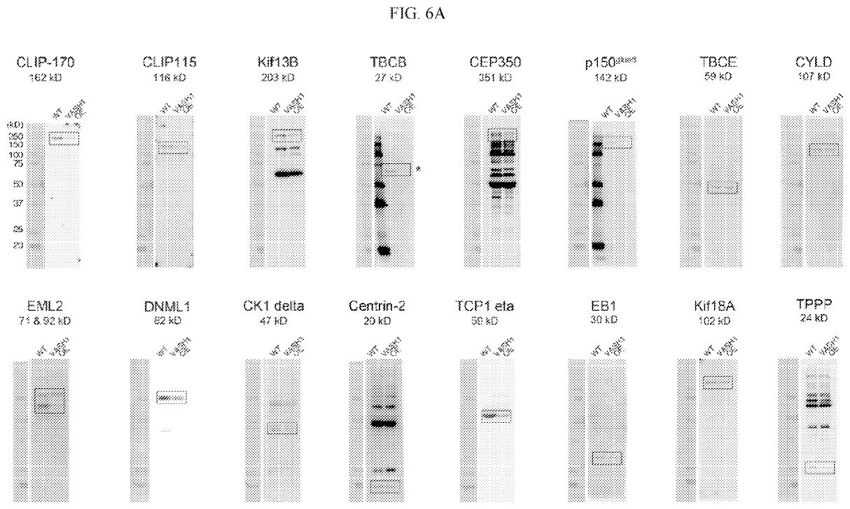

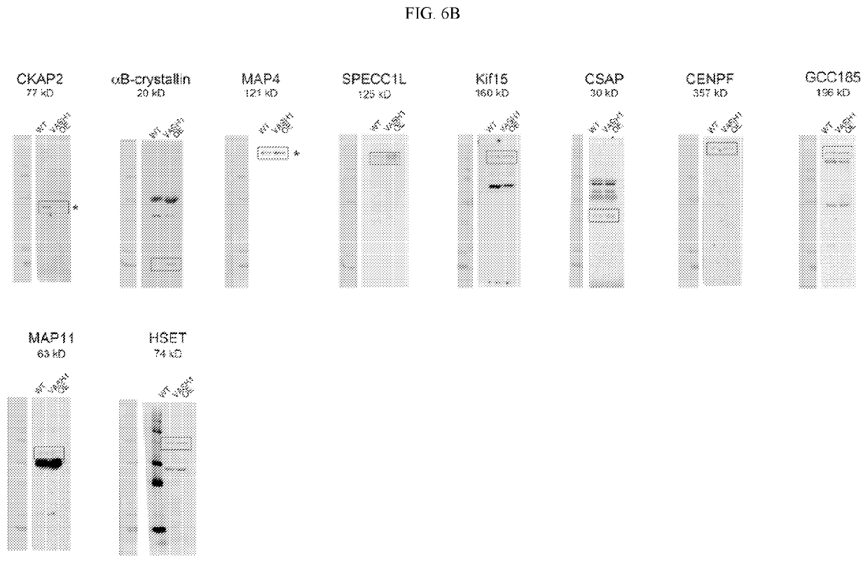

To isolate MAPs that bind to microtubules in a Y/ΔY-sensitive manner, we prepared MAPs from wild-type HeLa cells and HeLa cells engineered to overexpress VASH1-SVBP. As α-tubulin is largely tyrosinated in HeLa cells (5), we utilized a stable cell line overexpressing VASH1/SVBP (VASH OE) for stoichiometric detyrosination (FIG. 1B and FIG. 5), thereby generating a binary screening platform to identify proteins that associate with Y- versus ΔY-microtubules (FIG. 1A). Microtubules were assembled in HeLa or VASH1 OE cell extracts, sedimented by centrifugation, and the proteins in the pellet (FIG. 1C) were tandem mass-tagged (TMT) (6) and subjected to mass spectrometry (MS). The microtubule interactomes in Y versus ΔY cell extracts demonstrated that CAP-Gly-containing proteins were enriched in the Y-microtubule pellet (FIG. 1D, blue dots), a result that was also validated by immunoblotting (FIG. 1E and FIG. S2). Additional proteins enriched in Y-microtubule pellets include CKAP2, TCP1-eta, and a splice variant of echinoderm microtubule-associated protein like 2 (EML2-S) (FIG. 1, D and F; and FIG. 6). Our screening pipeline also identified candidate readers of ΔY-microtubules including CSAP, SPECC1L, and αB-crystallin (FIG. 1G and FIG. 6). We observed good agreement between enrichment scores obtained by TMT analysis and quantitative immunoblotting (FIG. 1H), suggesting that our pipeline to identify readers of Y- versus ΔY-microtubules is sensitive and robust.

### Example 3

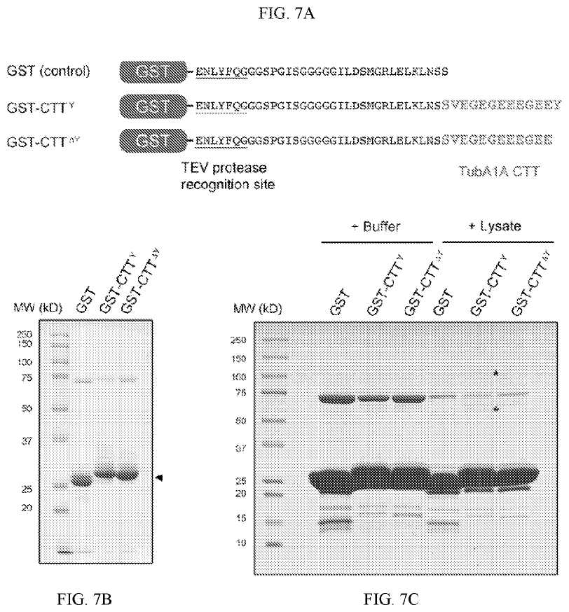

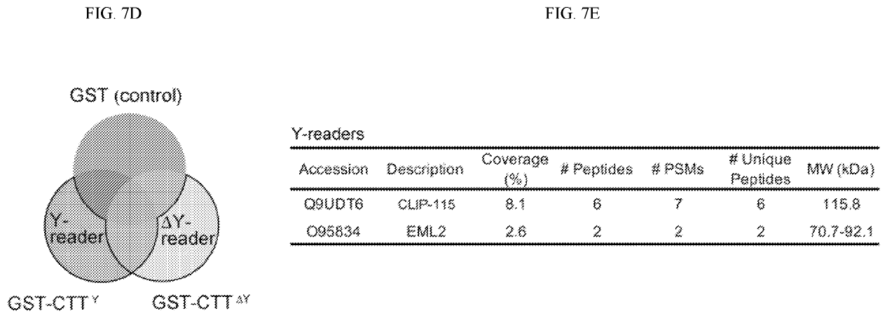

We used an orthogonal approach to identify proteins that preferentially bind Y- versus ΔY microtubules, via a mechanism that only requires the CTT. Sequences corresponding to the full length or ΔY α-tubulin CTT of TubA1A were fused to GST (FIG. 7A) and the resulting fusion proteins immobilized on glutathione agarose. These resins were used as affinity reagents to capture interacting proteins from CHL-1 cell lysates; CHL-1 cells express high levels of ΔY-tubulin (3), suggesting that these cells may contain readers of both Y- and ΔY-microtubules. Again, mass spectrometry revealed a CAP-Gly protein (CLIP-115) to be Y-microtubule-specific, consistent with previous findings showing that CAP-Gly domains can interact with tubulin solely through the α-tubulin CTT (7). Although we were not able to identify MAPs that specifically associate with the ΔY-α-tubulin CTT, we again detected EML2 as a protein that preferentially binds Y-α-tubulin CTT (FIG. 7).

Echinoderm microtubule-associated protein (EMAP) is the founding member of a large family of MAPs and associates with microtubules during interphase and mitosis in urchin embryos (8). Mammals express 6 EML proteins (9), with EML1, 2, 3, and 4 sharing similar domain organization (FIG. 2A) comprising N-terminal coiled-coil and basic regions that promote trimerization and microtubule binding (10, 11), respectively, and a C-terminal array of WD (tryptophan-aspartate) repeats. Structural studies of EML1 show that the WD repeats fold into 13 individual β-sheets that form the blades of 2 β-propeller structures (12). The first β-propeller is assembled from 7 contiguous WD repeats, but the second β-propeller is atypical in that its 12th blade is partially formed from a hydrophobic EML protein (HELP) motif that is located upstream of sequences that form the first D-propeller (FIG. 2A). Together, the two-β-propeller architecture is referred to as a TAPE (tandem atypical propeller in EML) domain. In humans, EML1 and EML2 appear to be expressed ubiquitously, and for all EMLs, there is clear evidence of differential splicing (13).

### Example 4

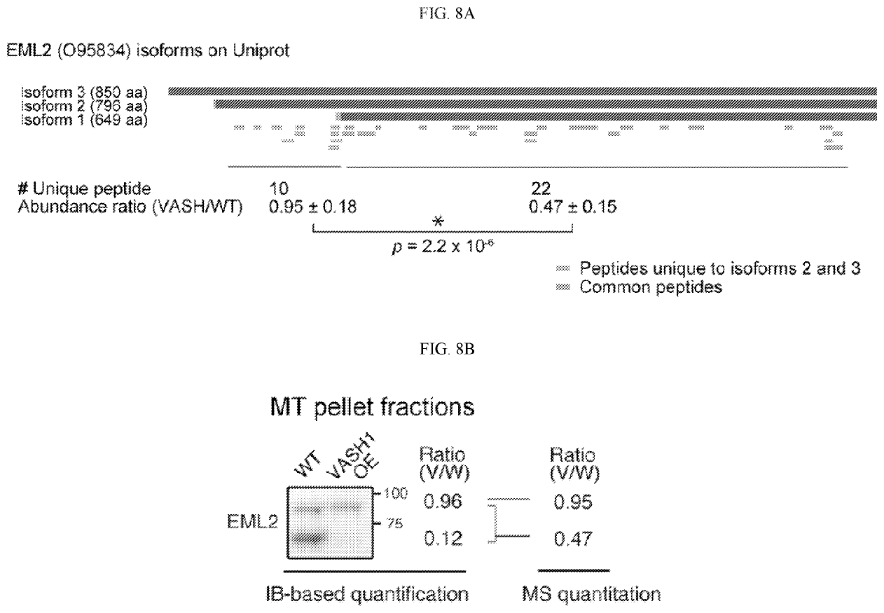

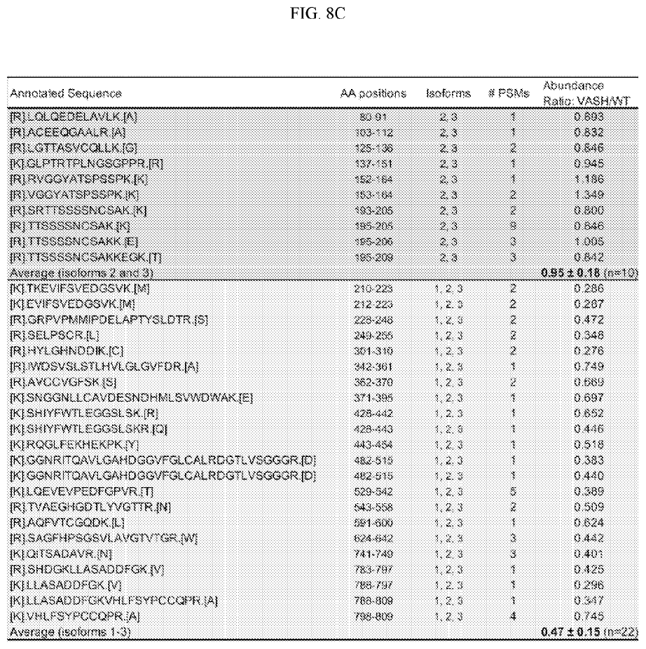

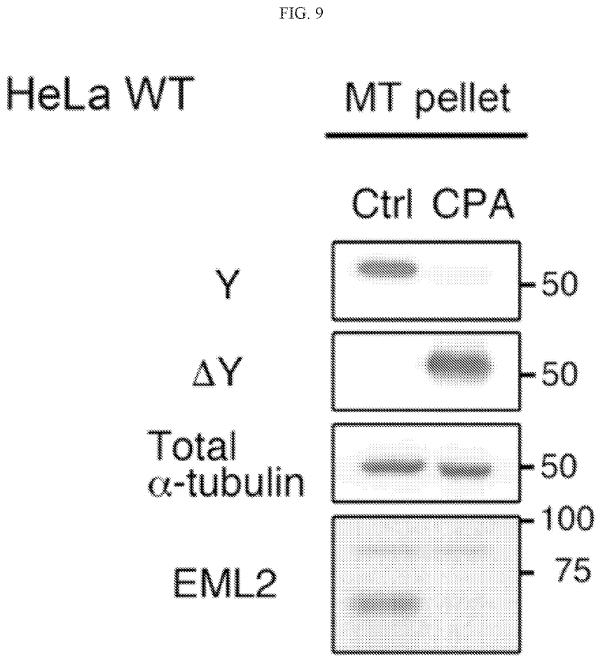

Although we detected EML1, 2, 3 and 4 in our Y- and ΔY-interactomes, only peptides corresponding to EML2 were detected as being Y-microtubule specific (FIG. 2B; FIG. 8, A and C). Further analysis suggested that peptides that correspond to the shortest EML2 isoform are enriched with Y-microtubules. To identify which isoform of EML2 can function as a Y-reader, we immunoblotted the Y/ΔY microtubule pellet fractions with an antibody specific to EML2 TAPE domain. The higher molecular weight (MW) band, which corresponds to full-length EML2 (EML2-L), was found in both Y- and ΔY-pellets whereas the lower MW band, which corresponds to isoform 1 (EML2-S, produced by alternative splicing), showed preferential binding to Y-microtubules (FIG. S4B). Association of EML2-S with Y-microtubules was reproduced when using ΔY-microtubules prepared with carboxypeptidase A (CPA; FIG. 9). EML2-S is expected to be a monomer in solution, as it only contains the TAPE domain of EML2 (FIG. 2A)

### Example 5

We generated epitope-tagged forms of EML1, EML2-L, EML2-S, EML3 and EML4 to validate EML2-S as a Y-microtubule-specific reader, opting to use the PA-tag (14) since GFP- or SNAP-tags negatively impacted the ability of EML2-S to bind microtubules (FIG. 10). In microtubule co-sedimentation assays using lysates from cells expressing doxycycline-induced PA-tagged EML proteins, only EML2-S showed microtubule binding that was sensitive to CPA (FIG. 2C). In HeLa cells, immunofluorescence revealed that EML2-L and EML2-S decorated microtubules similarly during interphase (FIG. 2D and FIG. 11). However, in cells undergoing cytokinesis, EML2-S was largely excluded from the midbody, a structure known to contain high levels of ΔY-microtubules (FIG. 2, E and F), consistent with its preference for binding Y-microtubules

### Example 6

To verify that the Y-microtubule preference of EML2-S is direct, i.e., not mediated by other protein factors, we examined the ability of recombinant EML2-S to bind Y- or ΔY-microtubules in vitro. EML2-S was expressed in and purified from insect cells and assessed for its ability to co-sediment with Y-microtubules or ΔY-microtubules generated with CPA (FIG. 12). EML2-S showed increased binding to Y-microtubules as compared to ΔY-microtubules (FIG. 2, G and H), allowing us to conclude that EML2-S is a bona fide reader of Y-microtubules, similar to CAP-Gly domain containing proteins.

### Example 7

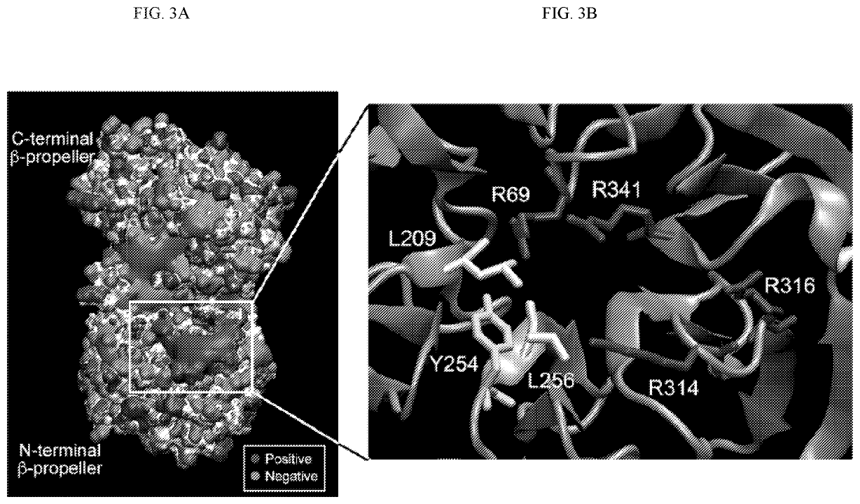

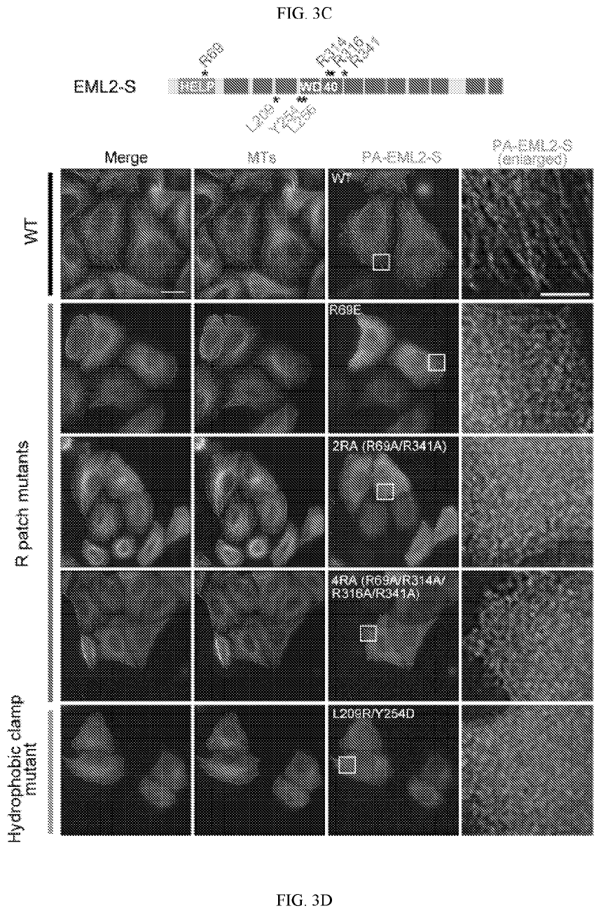

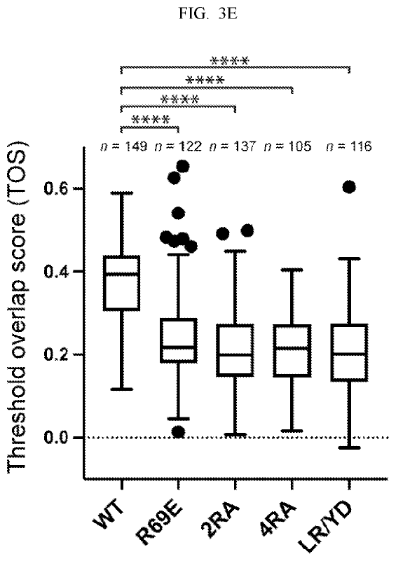

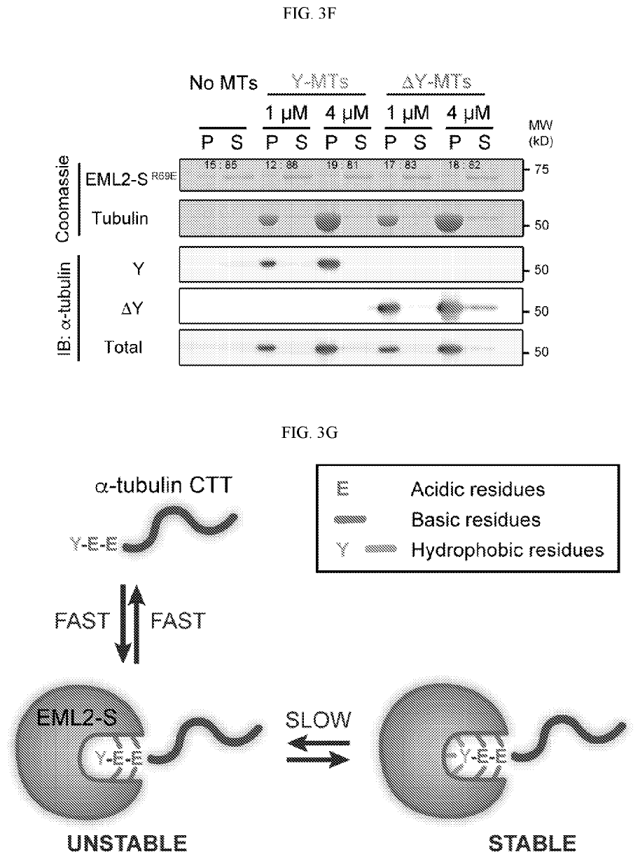

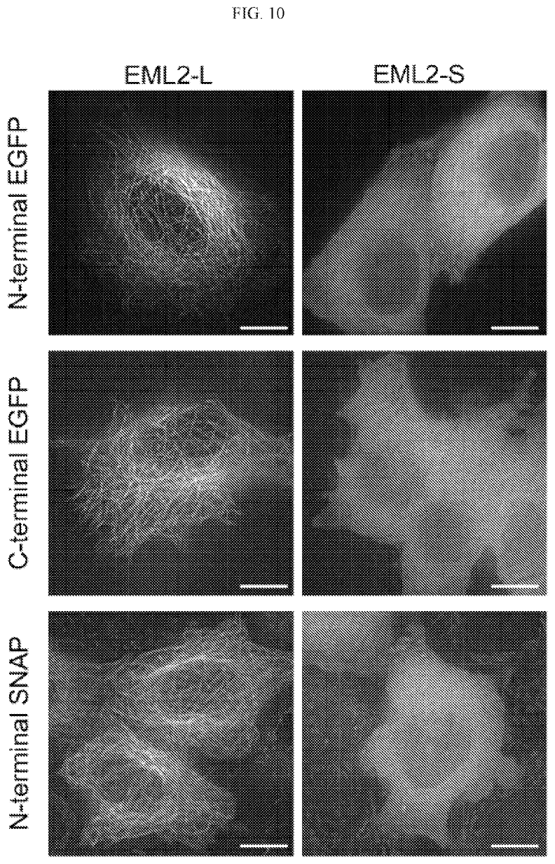

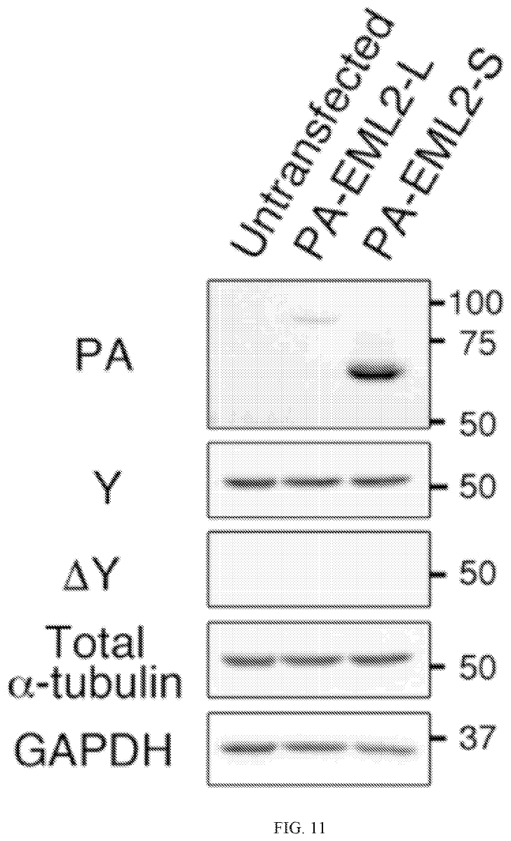

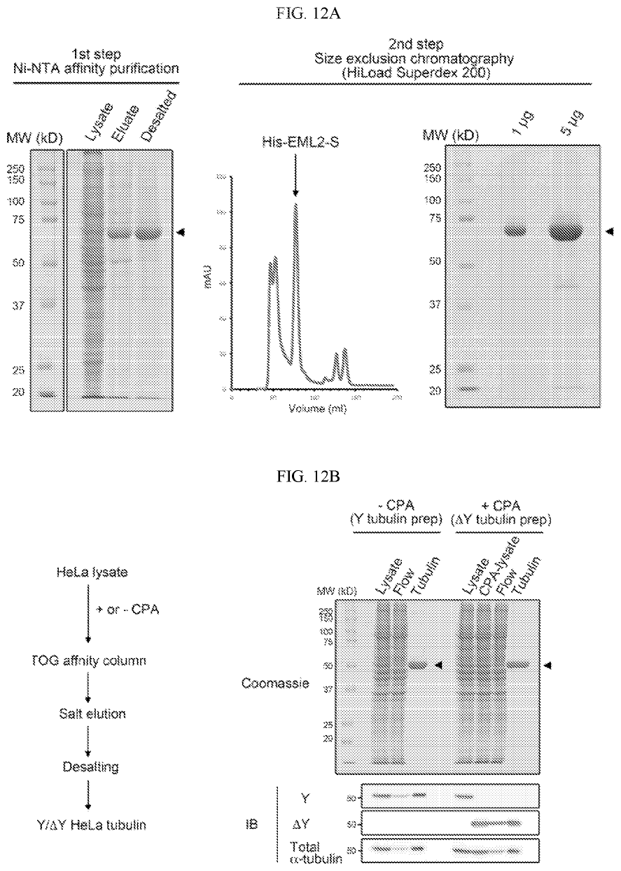

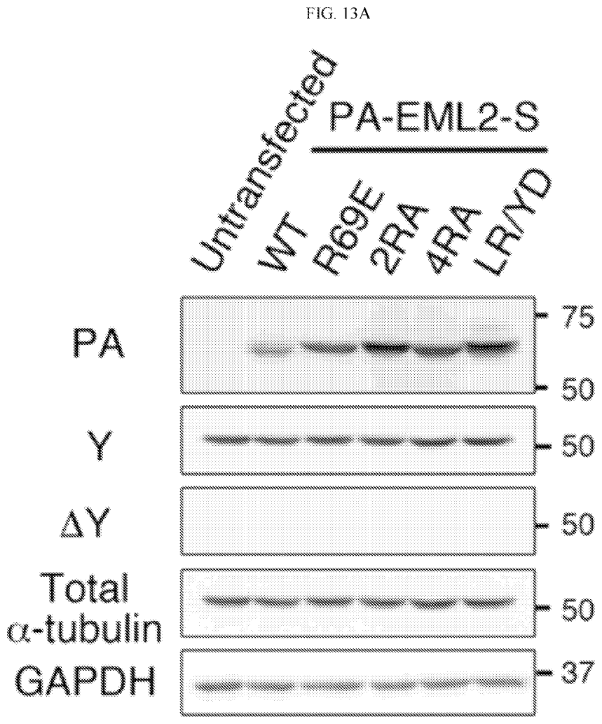

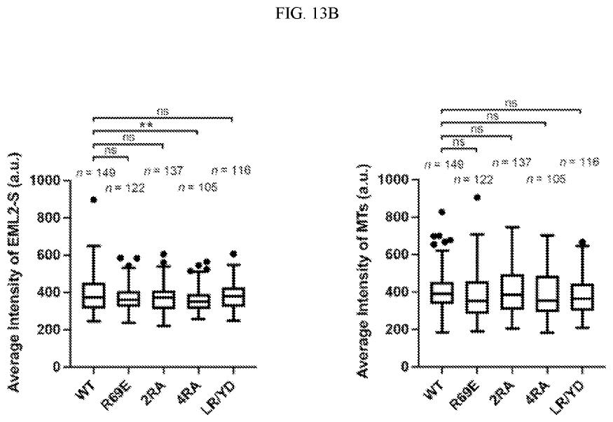

The CTT of tubulin is rich in glutamate residues and its binding with MAPs is typically characterized by strong electrostatic interactions. To identify where and how the CTT could interact with EML2-S, we constructed a homology model for EML2-S and then performed Poisson-Boltzmann calculations to look at the protein electrostatics. When the electrostatic potential is mapped on to the protein surface, we observed a large, highly basic patch in the N-terminal β-propeller domain of EML2 (blue surface in FIG. 3A). The potential in this region largely comes from four basic residues: R69, R314, R316 and R341 (FIG. 3B). At the end of this “R-patch” are a group of aromatic/hydrophobic residues—L209, Y254 and L256, resulting in a putative binding site reminiscent of that found in CAP-Gly domains (7, 15). The R-patch would be predicted to have very strong interactions with the glutamates of the CTT, and the C-terminal tyrosine would have both hydrophobic packing and ring-stacking interactions. To test this, we performed mutagenesis of the residues in EML2-S (FIG. 3C) and analyzed the ability of these mutants to bind interphase microtubules in HeLa cells. The EML2-S charge-reversal mutant R69E failed to interact with microtubules in cells, as did 2RA (R69A/R341A) and 4RA (R69A/R314A/R316A/R341A) mutants. An EML2-S double mutant in which hydrophobic residues L209 and Y254 were changed to R and D residues, respectively, also failed to bind 15 microtubules in cells (FIG. 3, D and E; and FIG. 13). These data support a model in which the N-terminal β-propeller of EML2-S binds the CTT of α-tubulin using a combination of electrostatic and hydrophobic interactions. To further investigate this, we expressed and purified recombinant EML2-SR69E and examined its ability to bind Y- or ΔY-microtubules in vitro using a cosedimentation assay. Similar to its behavior in cells, EML2-SR69E showed only weak interaction with either Y- or ΔY-microtubules (FIG. 3F). Collectively, these data suggest that the interaction of EML2-S with microtubules is initiated by weak electrostatic interactions and is stabilized by interaction of the C-terminal Y with L209 and Y254 (FIG. 3G). With just the electrostatic interaction, we contemplate that the detyrosinated CTT is unable to stay in place (FIG. 2, G and H). A similar mechanism underlies the interaction of the 2nd CAP-Gly domain of CLIP170 with the ΔY-αCTT (15).

### Example 8

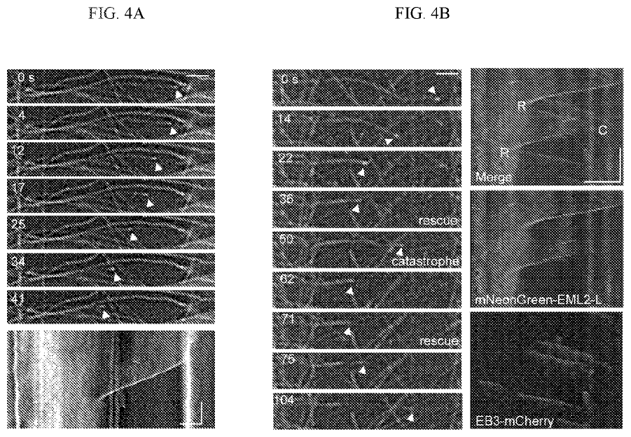

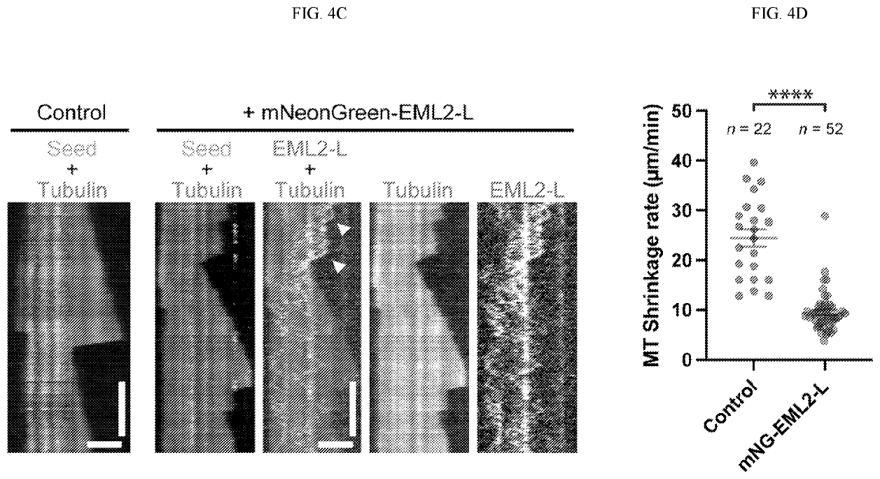

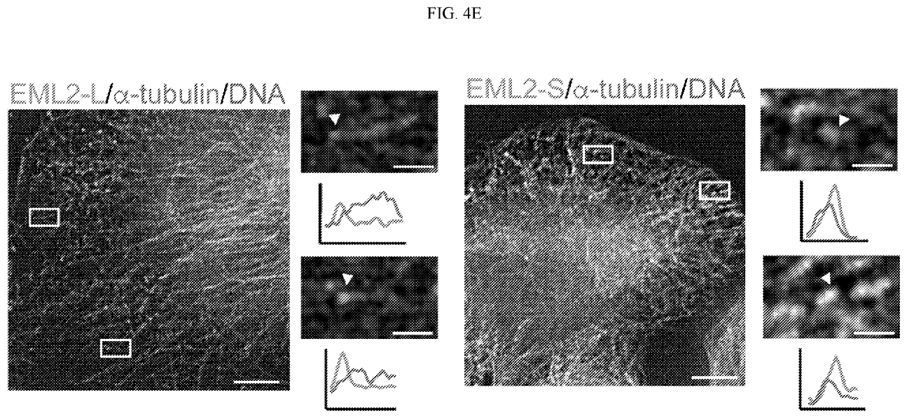

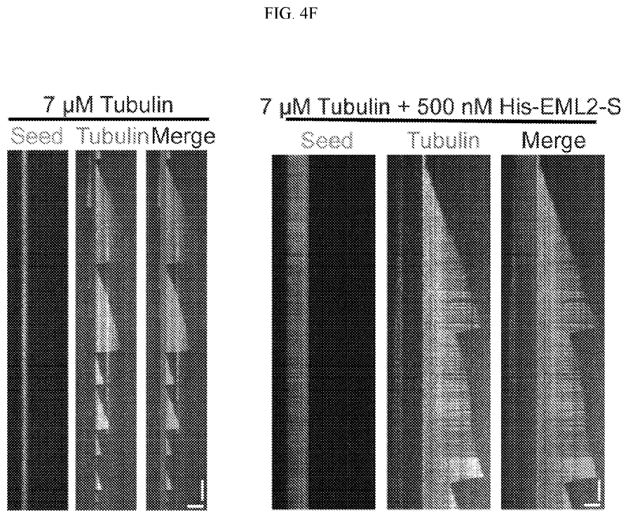

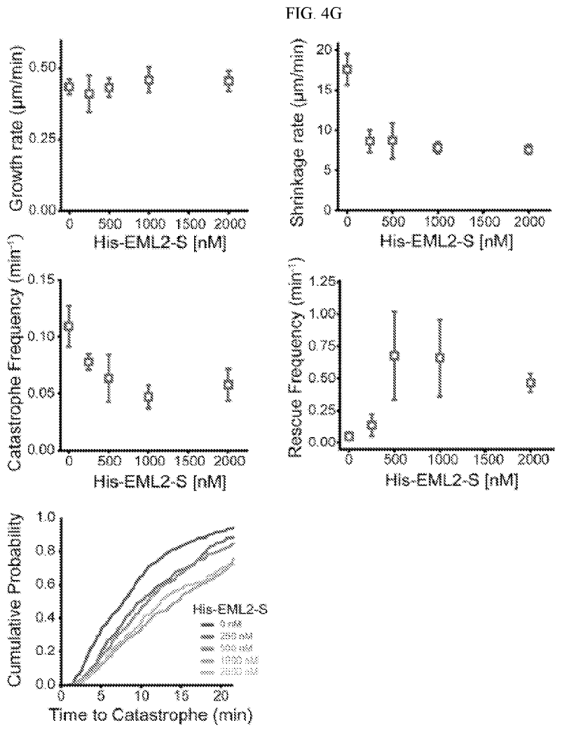

To understand the functional significance of EML2's ability to interact with Y-MTs, we proceeded by analyzing the localization of EML2 in cells. Since EML2-S is not readily fused with a large tag without disrupting its ability to bind microtubules (FIG. 10), we began by imaging mNeonGreen 30 (mNG) fused to the long isoform, EML2-L. mNG-EML2-L localized to the microtubule lattice but also at ends of microtubules that were undergoing growth or shortening (FIG. 4A and movie 5). To further investigate this, we examined the localizations of mNG-EML2-L and EB3-mCherry, a protein that tracks growing microtubule ends, in HeLa cells. Strikingly, mNG-EML2-L localized to microtubule ends that were not decorated with EB3-mCherry, indicating that mNG-EML2-L concentrates at the tips of shortening microtubules (FIG. 4B). As a first step to understand the mechanism by which EML2-L tracks shortening microtubule ends, we reconstituted microtubule dynamics by growing microtubules from stable GMPCPP microtubule “seeds” and visualized their dynamics in the absence or presence of cell lysates containing mNGEML2-L (17). mNG-EML2-L underwent one-dimensional diffusion along the surface of the microtubule and became enriched at shrinking microtubule ends (FIG. 4C). Accumulation of mNGEML2-L at the shrinking plus ends resulted in a significantly lower depolymerization rate (Control, 24.5+/−1.7 μm/min [n=22]; With mNG-EML2-L, 9.5+/−0.54 μm/min [n=52][mean+/−SE]; FIG. 4D), implicating EML2-L as a regulator of microtubule dynamics.

### Example 9

To determine if EML2-S can also localize to shrinking microtubule ends and modify their depolymerization rate, we first examined the localization of PA-tagged EML2-L and EML2-S by indirect immunofluorescence in cells treated with nocodazole, a drug that induces microtubule disassembly. Brief exposure (16 seconds) of cells to nocodazole caused thinning and fragmentation of interphase microtubule arrays (FIG. 4E). Both EML2-L and EML2-S, detected with a PA-tag antibody, localized at microtubule tips suggesting that both isoforms localize to the plus ends of shortening microtubules.

### Example 10

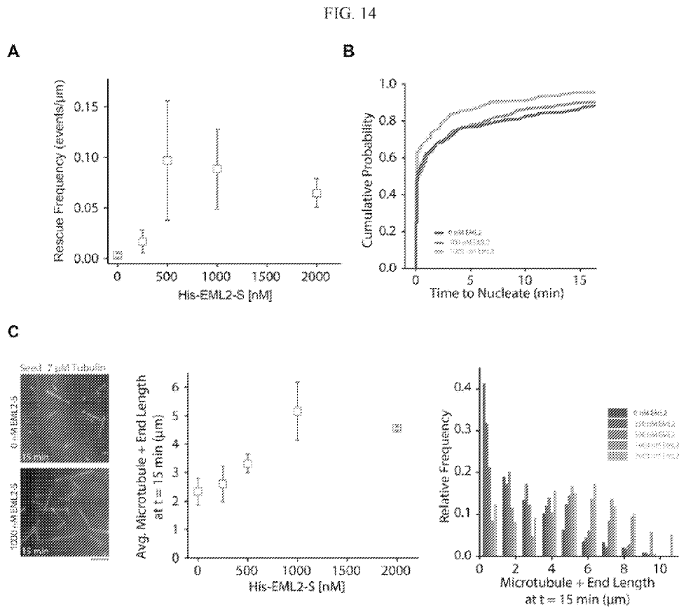

To examine the influence of EML2-S on microtubule dynamics, we measured microtubule dynamics in the absence and presence of purified EML2-S protein In control conditions, microtubules exhibited typical in vitro dynamics with fast shrinkage rates, and low rescue frequencies (FIG. 4, F and G; and FIG. 14A) (18). Addition of purified EML2-S resulted in a concentration-dependent reduction in microtubule shrinkage rate and catastrophe frequency as well as a dramatic increase in rescue frequency (FIG. 4G). Microtubule growth rates were not affected by EML2-S and templated nucleation rates increased only minimally (FIG. 14B). Together, these changes in microtubule dynamics lead to increased average microtubule length overtime (FIG. 14C).

### Example 11

The screening described above identified new readers of Y- versus ΔY-microtubules. The WD-repeats of EML2 form a Y-αCTT-binding module. Humans encode 921 WD repeat proteins that carry out a broad range of biological functions (19). A subset of these are MAPs and motor proteins, e.g., KIF21B (20), WDR47 (21) and LRRK2 (22), but the structural mechanism(s) that enables microtubule association is not known in any of these cases. We show here that the CAP-Gly and TAPE domains share general features that allow them to bind the Y-αCTT. Like EML2-S, some CAP-Gly proteins, e.g., cytoplasmic linker proteins (CLIPs), use a combination of electrostatic and hydrophobic interactions to engage the Y-αCTT. In the case of CAP-Gly domains, synergy between electrostatic and hydrophobic interactions dramatically increase the strength of Y-αCTT interactions, and similar to EML2-S charge-neutralization mutants of residues that coordinate ionic interactions with glutamates in the Y-αCTT significantly reduce CAP-Gly:Y-αCTT interaction strength (15). Intriguingly, CAP-Gly domains use their ability to bind the Y-αCTT to track growing microtubule plus ends, but EML2, instead uses its Y-αCTT recognition motif to track shrinking microtubule ends and to regulate microtubule dynamics.

To examine if EML proteins differ in their functional properties, we fused EML1, 3 and 4 to mNG, and imaged them by live cell microscopy. All proteins localized to microtubules, but only mNG-EML1 showed an enrichment on microtubule ends. Interestingly, mNG-EML3 uniquely caused microtubule curling and fragmentation. Therefore, EML1-4 all engage microtubules, but each isoform has unique activities when bound to microtubules.

TABLE 1
List of oligonucleotides used in the experiments

| | SEQ ID | | |
|---|---|---|---|
| Name | NO: | Sequence (5′-3′) | Used for |
| TH460 | 63 | AGCACCCAGTCCAAGCTGAGCAAAGAC | A207K mutagenesis to |
| | | | generate monomeric EGFP |
| TH511 | 64 | ACCCTCTCCTTCAACCGATGAATTAAGCTT | Insertion of TubA1A CTT to |
| | | GAGCTC | pGEX-KGT |
| TH512 | 65 | gaggaagaaggagaggaatactgactgactgacgatctgc | Insertion of TubA1A CTT(Y) to |
| | | ct | pGEX-KGT |
| TH513 | 66 | gaggaagaaggagaggaatgactgactgacgatctgcct | Insertion of TubA1A CTT(dY) to |
| | | | pGEX-KGT |
| TH607 | 67 | gatctcgagctcaagcttcgATGCTGGAACGAAGG | Cloning EML2 partial cDNA into |
| | | GCGTTGCTATG | pPA-EGFP-C1 |
| TH607 | 68 | gatctcgagctcaagcttcgATGCTGGAACGAAGG | Cloning EML2-L into |
| | | GCGTTGCTATG | pmNeonGreen-C1 |
| TH608 | 69 | cgcggtaccgtcgactgcagaattcTTGCGGCCGCC | Cloning EML2 partial cDNA into |
| | | CTGGTCCAC | pPA-EGFP-C1 |
| TH609 | 70 | GAGGATGGCTCCGTGAAAATGTTCCTG | Correction of L222 to V |
| TH610 | 71 | ggtggaccagggcggccgcAAgGTCAGCCGCCTG | Addition of EML2 C-terminal |
| | | GGC | 155 aa |
| TH611 | 72 | cgcggtaccgtcgactgcagTCAGACCACCCGCCA | Addition of EML2 C-terminal |
| | | CTG | 155 aa |
| TH640 | 73 | gatctcgagctcaagcttcgATGAGTAGCTTTGGAG | Cloning EML2-S into pPA- |
| | | CTGGCAAAACCAAAGAAG TTATC | EGFP-C1 |
| TH641 | 74 | taccgcgggcccgggatccaATGGGCGTTGCCAT | Cloning PA-EMLs into pEM791 |
| | | GCCA | |
| TH642 | 75 | ctgcacctgagtgtttacttTCAGACCACCCGCCAC | Cloning PA-EML2-L/S into |
| | | TG | pEM791 |
| TH647 | 76 | gtgccgaagatgatgtggtgCTGGAACGAAGGGCG | Cloning EML2-L into pPA- |
| | | TTG | EGFP-N1 |
| TH648 | 77 | ccgcctgaaccgcctccaccGACCACCCGCCACT | Cloning EML2-L/S into pPA- |
| | | GTAG | EGFP-N1 |
| TH649 | 78 | gtgccgaagatgatgtggtgAGTAGCTTTGGAGCT | Cloning EML2-S into pPA- |
| | | GGC | EGFP-N1 |
| TH655 | 79 | acgtaggcctttgaattccgTCAGACCACCCGCCAC | Cloning EML2-S into pFastBac- |
| | | TG | HTA |
| TH656 | 80 | tgtattttcagggcgccatgagtagctttggagctggcaaaac | Cloning EML2-S into pFastBac- |
| | | | HTA |
| TH657 | 81 | tgggctgggtcctgcaggcgtgggcgttgccatgccag | Cloning EML2-L/S into pSNAPf |
| TH658 | 82 | taacctcgagtttaaacgcgttagaccacccgccactgtag | Cloning EML2-L/S into pSNAPf |
| TH661 | 83 | CAGCACCACATCATCTTCGGCAC | Removal of EGFP from pPA- |
| | | | EGFP-C1-EML2-L |
| TH662 | 84 | GAACGAAGGGCGTTGCTATGGCA | Removal of EGFP from pPA- |
| | | | EGFP-C1-EML2-L |
| TH664 | 85 | CACCACATCATCTTCGGCACCTG | Removal of EGFP from pPA- |
| | | | EGFP-C1-EML2-S |
| TH665 | 86 | agtagctttggagctGGCAAAACC | Removal of EGFP from pPA- |
| | | | EGFP-C1-EML2-S |
| TH670 | 87 | caccacatcatcttcggcacc | Amplifying pPA vector |
| | | | backbone |
| TH671 | 88 | tccggactcagatctcgagctc | Amplifying pPA vector |
| | | | backbone |
| TH672 | 89 | ggtgccgaagatgatgtggtggaggacggcttctccagctac | Cloning EML1 cDNA into pPA- |
| | | | EGFP-C1 |
| TH674 | 90 | ggtgccgaagatgatgtggtggacggggccgcggggcccg | Cloning EML3 cDNA into pPA- |
| | | gt | EGFP-C1 |
| TH675 | 91 | agctcgagatctgagtccggatcaaacgtcgagggaggag | Cloning EML3 cDNA into pPA- |
| | | gcgggggacaggg | EGFP-C1 |
| TH676 | 92 | ggtgccgaagatgatgtggtggacggtttcgccggcagtctc | Cloning EML4 cDNA into pPA- |
| | | | EGFP-C1 |
| TH677 | 93 | agctcgagatctgagtccggattaggacgagggcgaagggt | Cloning EML4 cDNA into pPA- |
| | | c | EGFP-C1 |
| TH702 | 94 | agctcgagatctgagtccggaCtaaatgacgcgccactgca | Cloning EML1 cDNA into pPA- |
| | | tg | EGFP-C1 |
| TH703 | 95 | ctgcacctgagtgtttacttCTAAATGACGCGCCACT | Cloning PA-EML1 into pEM791 |
| | | GC | |
| TH704 | 96 | ctgcacctgagtgtttacttTCAAACGTCGAGGGAG | Cloning PA-EML3 into pEM791 |
| | | GAGG | |
| TH705 | 97 | ctgcacctgagtgtttacttTTAGGACGAGGGCGAA | Cloning PA-EML4 into pEM791 |
| | | GG | |
| TH745 | 98 | CAAATAAAGGTTGGCCGCGCAGTCTCGG | 2 R to A mutagenesis |
| TH746 | 99 | TTTGGCCCTGTGgcCACCGTGGCAGA | 2 R to A mutagenesis |
| TH747 | 100 | TGGAGGGGGCgcTGATgcGCGGGTGGTCC | Additional 2 R to A |
| | | TCT | mutagenesis |
| TH748 | 101 | CAAATAAAGGTTGGCCTCGCAGTCTCGG | R69E mutagenesis |
| TH750 | 102 | GGTGGCCACCCTTACAGCCTC | LR/YD mutagenesis |
| TH753 | 103 | GAAACCGAAGgATGTGCTGTGTGTG | LR/YD mutagenesis |
| TH780 | 104 | gatctcgagctcaagcttcgATGGAGGACGGCTTC | Cloning EML1 into |
| | | TCC | pmNeonGreen-C1 |
| TH781 | 105 | cgcggtaccgtcgactgcagCTAAATGACGCGCCA | Cloning EML1 into |
| | | CTG | pmNeonGreen-C1 |
| TH782 | 106 | gatctcgagctcaagcttcgATGGACGGGGCCGCG | Cloning EML3 into |
| | | GGG | pmNeonGreen-C1 |
| TH783 | 107 | cgcggtaccgtcgactgcagTCAAACGTCGAGGGA | Cloning EML3 into |
| | | GGAGGCGG | pmNeonGreen-C1 |
| TH784 | 108 | gatctcgagctcaagcttcgATGGACGGTTTCGCC | Cloning EML4 into |
| | | GGC | pmNeonGreen-C1 |
| TH785 | 109 | cgcggtaccgtcgactgcagTTAGGACGAGGGCG | Cloning EML4 into |
| | | AAGGG | pmNeonGreen-C1 |

TABLE 2
List of Plasmids used in the study

| Plasmid name | Used for |
|---|---|
| pPA-EGFP-C1-EML2-partial | Cloning |
| pPA-EGFP-C1-EML2-L with a mutation | Cloning |
| at Val222 | |
| pPA-EGFP-C1-EML2-L | Transient expression |
| pPA-EGFP-C1-EML2-S | Transient expression |
| pPA-EGFP-N1-EML2-L | Transient expression |
| pPA-EGFP-N1-EML2-S | Transient expression |
| pSNAPf-EML2-L | Transient expression |
| pSNAPf-EML2-S | Transient expression |
| pPA-EML2-L | Transient expression |
| pPA-EML2-S | Transient expression |
| pPA-EML2-S R69E | Transient expression |
| pPA-EML2-S R69A/R341A (2RA) | Transient expression |
| pPA-EML2-S R69A/R314A/R316A/ | Transient expression |
| R341A (4RA) | |
| pPA-EML2-S L209R/Y254D (LR/YD) | Transient expression |
| pPA-EML1 | Cloning |
| pPA-EML3 | Cloning |
| pPA-EML4 | Cloning |
| pEM791-PA-EML1 | Establishing knock-in cell line |
| pEM791-PA-EML2-L | Establishing knock-in cell line |
| pEM791-PA-EML2-S | Establishing knock-in cell line |
| pEM791-PA-EML3 | Establishing knock-in cell line |
| pEM791-PA-EML4 | Establishing knock-in cell line |
| pmNeonGreen-C1-EML1 | Transient expression |
| pmNeonGreen-C1-EML2-L | Transient expression |
| pmNeonGreen-C1-EML3 | Transient expression |
| pmNeonGreen-C1-EML4 | Transient expression |
| pFastBac-EML2-S | Baculovirus |
| pFastBac-EML2-S R69E | Baculovirus |
| pGEX-KGT | Bacterial expression |
| pGEX-KGT-aCTT(Y) | Bacterial expression |
| pGEX-KGT-aCTT(DY) | Bacterial expression |

TABLE 3
List of antibodies used in the immunofluorescence and immunoblot analysis

| | | Source and | | | IB | IF |
|---|---|---|---|---|---|---|
| Type | Name | clone | Vendor | Cat # | conc. | conc. |
| Primary | Anti-tyrosinated | Rat | Accurate Chemical | YSRTMCA77G | 1:3,000 | N/A |
| | c- tubulin | Monoclonal, | and Scientific | | | |
| | | clone YL1//2 | | | | |
| | Anti- | Rabbit | RevMAb | 31-1335-00 | 0.1 μg/ml | 1 μg/ml |
| | detyrosinated | monoclonal, | Biosciences | | | |
| | c- tubulin | clone RM444 | | | | |
| | Anti-c-tubulin | Mouse | Millipore Sigma | 05-829 | 1:3,000 | N/A |
| | | monoclonal, | | | | |
| | | clone DM1c | | | | |
| | Anti-c-tubulin | Mouse | Sigma-Aldrich | F2,168 | N/A | 1:500 |
| | conjugated with | monoclonal, | | | | |
| | FITC | clone DM1c | | | | |
| | Anti-CLIP-170 | Rabbit | Novus | NBP1-85568 | 1:250 | N/A |
| | | polyclonal | Biologicals | | | |
| | Anti-CLIP-115 | Rabbit | Boster | A07287 | 1:500 | N/A |
| | Anti-Kif13B | Rabbit | Sigma-Aldrich | HPA025023 | 1:250 | N/A |
| | | polyclonal | | | | |
| | Anti-TBCB | Mouse | Santa Crutz | sc-390783 | 1:500 | N/A |
| | | monoclonal, | | | | |
| | | clone E-1 | | | | |
| | Anti-CEP350 | Rabbit | Novus | NB100-59810 | 1:500 | N/A |
| | | polyclonal | Biologicals | | | |
| | Anti-p150glued | Mouse | BD Transduction | 610474 | 1:400 | N/A |
| | | monoclonal | Laboratories | | | |
| | Anti-TBCE | Rabbit | Sigma-Aldrich | SAB1408635 | 1:500 | N/A |
| | | polyclonal | | | | |
| | Anti-CYLD | Mouse | Thermo Fisher | 43-7700 | 1:500 | N/A |
| | | monoclonal, | | | | |
| | | clone 733 | | | | |
| | Anti-EML2 | Rabbit | Sigma-Aldrich | HPA012757 | 1:200 | N/A |
| | | polyclonal | | | | |
| | Anti-EML2 | Rabbit | Proteintech | 13529-1-AP | 1:1,000 | N/A |
| | | polyclonal | | | | |
| | Anti-Dynamin | Rabbit | Thermo Fisher | PA1-16987 | 1:500 | N/A |
| | 1L | polyclonal | | | | |
| | Anti-CK1 delta | Goat | abcam | ab48031 | 1:500 | N/A |
| | | polyclonal | | | | |
| | Anti-Centrin | Rabbit | (34) | N/A | 1:1,000 | N/A |
| | Anti-TCP1 eta | Rabbit | Novus | NBP1-88144 | 1:250 | N/A |
| | | polyclonal | Biologicals | | | |
| | Anti-EB1 | Mouse | BD Transduction | 610535 | 1:1,000 | N/A |
| | | monoclonal | Laboratories | | | |
| | Anti-Kif18A | Rabbit | (35) | N/A | 1:1,000 | N/A |
| | Anti-TPPP | Goat | Thermo Fisher | PA5-19243 | 1:1,000 | N/A |
| | | polyclonal | | | | |
| | Anti-CKAP2 | Mouse | Santa Crutz | sc-398286 | 1:500 | N/A |
| | | monoclonal, | | | | |
| | | clone B-12 | | | | |
| | Anti-cB- | Rabbit | Thermo Fisher | PA1-16950 | 1:1,000 | N/A |
| | crystallin | polyclonal | | | | |
| | Anti-MAP4 | Rabbit | abcam | ab89650 | 1:800 | N/A |
| | | polyclonal | | | | |
| | Anti-SPECC1L | Rabbit | Proteintech | 25390-1-AP | 1:500 | N/A |
| | | polyclonal | | | | |
| | Anti-Kif15 | Rabbit | (36) | N/A | 1:2,000 | N/A |
| | Anti-CSAP | Rabbit | (37) | N/A | 1:200 | N/A |
| | Anti-CENPF | Rabbit | Novus | NBP2-56124 | 1:1,000 | N/A |
| | | | Biologicals | | | |
| | Anti-GCC185 | Rabbit | Sigma-Aldrich | HPA035849 | 1:500 | N/A |
| | | polyclonal | | | | |
| | Anti-MAP11 | Rabbit | Sigma-Aldrich | HPA019359 | 1:500 | N/A |
| | | polyclonal | | | | |
| | Anti-KifC1 | Mouse | Thermo Fisher | H00003833- | 1:500 | N/A |
| | (HSET) | monoclonal, | | M01 | | |
| | | clone 2B9 | | | | |
| | Anti-PA-tag | Rat | FUJIFILM Wako | 016-25861 | 1:5,000 | 1:500 |
| | | monoclonal, | Pure Chemical | | | |
| | | clone NZ-1 | | | | |
| | Anti-GAPDH | Mouse | Santa Crutz | sc-365062 | 1:2,000 | N/A |
| | | monoclonal, | | | | |
| | | clone G-9 | | | | |
| Secondary | Anti-rat Alexa | Goat | Thermo Fisher | A-11007 | N/A | 1:2,000 |
| | 594 | | | | | |
| | Anti-rabbit | Goat | Thermo Fisher | A-21245 | N/A | 1:2,000 |
| | Alexa 647 | | | | | |
| | Anti-rat Alexa | Goat | Thermo Fisher | A-21096 | 1:10,000 | N/A |
| | 680 | | | | | |
| | Anti-rabbit | Goat | Jackson | 711-625-152 | 1:5,000 | N/A |
| | Alexa 680 | | ImmunoResearch | | | |
| | | | Laboratorie | | | |
| | Anti-mouse | Goat | Thermo Fisher | A-21036 | 1:10,000 | N/A |
| | Alexa 700 | | | | | |
| | Anti-rabbit | Goat | Thermo Fisher | A-21038 | 1:10,000 | N/A |
| | Alexa 700 | | | | | |
| | Anti-goat | Donkey | Thermo Fisher | A-11370 | 1:10,000 | N/A |
| | Alexa 790 | | | | | |
| | Anti-mouse | Goat | Thermo Fisher | SA5-10176 | 1:10,000 | N/A |
| | DyLight 800 | | | | | |
| | Anti-rabbit | Goat | LI-COR | 926-32211 | 1:10,000 | N/A |
| | IRDye 800CW | | | | | |

All-in-one expression vector comprising EML2 and EB3
(SEQ ID NO: 7)
TAGTTATTAATAGTAATCAATTACGGGGTCATTAGTTCATAGCCCATATATGGAGTT
CCGCGTTACATAACTTACGGTAAATGGCCCGCCTGGCTGACCGCCCAACGACCCCCG
CCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGT
GTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGT
ATTAGTCATCGCTATTACCATGGTGATGCGGTTTTGGCAGTACATCAATGGGCGTGG
ATAGCGGTTTGACTCACGGGGATTTCCAAGTCTCCACCCCATTGACGTCAATGGGAG
TTTGTTTTGGCACCAAAATCAACGGGACTTTCCAAAATGTCGTAACAACTCCGCCCC
ATTGACGCAAATGGGCGGTAGGCGTGTACGGTGGGAGGTCTATATAAGCAGAGCTG
GTTTAGTGAACCGTCAGATCCGCTAGCGCTACCGGTCGCCACCATGGCCGTCAATGT
GTACTCCACATCTGTGACCAGTGAAAATCTGAGTCGCCATGATATGCTTGCATGGGT
CAACGACTCCCTGCACCTCAACTATACCAAGATAGAACAGCTTTGTTCAGGGGCAGC
CTACTGCCAGTTCATGGACATGCTCTTCCCCGGCTGTGTGCACTTGAGGAAAGTGAA
GTTCCAGGCCAAACTAGAGCATGAATACATCCACAACTTCAAGGTGCTGCAAGCAG
CTTTCAAGAAGATGGGTGTTGACAAAATCATTCCTGTAGAGAAATTAGTGAAAGGA
AAATTCCAAGATAATTTTGAGTTTATTCAGTGGTTTAAGAAATTCTTTGACGCAAAC
TATGATGGAAAGGATTACAACCCTCTGCTGGCGCGGCAGGGCCAGGACGTAGCGCC
ACCTCCTAACCCAGGTGATCAGATCTTCAACAAATCCAAGAAACTCATTGGCACAGC
AGTTCCACAGAGGACGTCCCCCACAGGCCCAAAAAACATGCAGACCTCTGGCCGGC
TGAGCAATGTGGCCCCCCCCTGCATTCTCCGGAAGAATCCTCCATCAGCCCGAAATG
GCGGCCATGAGACTGATGCCCAAATTCTTGAACTCAACCAACAGCTGGTGGACTTGA
AGCTGACAGTGGATGGGCTGGAGAAGGAACGTGACTTCTACTTCAGCAAACTTCGT
GACATCGAGCTCATCTGCCAGGAGCATGAAAGTGAAAACAGCCCTGTTATCTCAGG
CATCATTGGCATCCTCTATGCCACAGAGGAAGGATTCGCACCCCCTGAGGACGATG
AGATTGAAGAGCATCAACAAGAAGACCAGGACGAGTACCTGGATCCCGCCACCATG
GTGAGCAAGGGCGAGGAGGATAACATGGCCATCATCAAGGAGTTCATGCGCTTCAA
GGTGCACATGGAGGGCTCCGTGAACGGCCACGAGTTCGAGATCGAGGGCGAGGGCG
AGGGCCGCCCCTACGAGGGCACCCAGACCGCCAAGCTGAAGGTGACCAAGGGTGGC
CCCCTGCCCTTCGCCTGGGACATCCTGTCCCCTCAGTTCATGTACGGCTCCAAGGCCT
ACGTGAAGCACCCCGCCGACATCCCCGACTACTTGAAGCTGTCCTTCCCCGAGGGCT
TCAAGTGGGAGCGCGTGATGAACTTCGAGGACGGCGGCGTGGTGACCGTGACCCAG
GACTCCTCCCTGCAGGACGGCGAGTTCATCTACAAGGTGAAGCTGCGCGGCACCAA
CTTCCCCTCCGACGGCCCCGTAATGCAGAAGAAGACCATGGGCTGGGAGGCCTCCT
CCGAGCGGATGTACCCCGAGGACGGCGCCCTGAAGGGCGAGATCAAGCAGAGGCT
GAAGCTGAAGGACGGCGGCCACTACGACGCTGAGGTCAAGACCACCTACAAGGCCA
AGAAGCCCGTGCAGCTGCCCGGCGCCTACAACGTCAACATCAAGTTGGACATCACC
TCCCACAACGAGGACTACACCATCGTGGAACAGTACGAACGCGCCGAGGGCCGCCA
CTCCACCGGCGGCATGGACGAGCTGTACAAGTAACCCCCCCCTAACGTTACTGGCCG
AAGCCGCTTGGAATAAGGCCGGTGTGCGTTTGTCTATATGTTATTTTCCACCATATTG
CCGTCTTTTGGCAATGTGAGGGCCCGGAAACCTGGCCCTGTCTTCTTGACGAGCATT
CCTAGGGGTCTTTCCCCTCTCGCCAAAGGAATGCAAGGTCTGTTGAATGTCGTGAAG
GAAGCAGTTCCTCTGGAAGCTTCTTGAAGACAAACAACGTCTGTAGCGACCCTTTGC
AGGCAGCGGAACCCCCCACCTGGCGACAGGTGCCTCTGCGGCCAAAAGCCACGTGT
ATAAGATACACCTGCAAAGGCGGCACAACCCCAGTGCCACGTTGTGAGTTGGATAG
TTGTGGAAAGAGTCAAATGGCTCTCCTCAAGCGTATTCAACAAGGGGCTGAAGGAT
GCCCAGAAGGTACCCCATTGTATGGGATCTGATCTGGGGCCTCGGTGCACATGCTTT
ACATGTGTTTAGTCGAGGTTAAAAAACGTCTAGGCCCCCCGAACCACGGGGACGTG
GTTTTCCTTTGAAAAACACGATGATAATATGGTGAGCAAGGGCGAGGAGGATAACA
TGGCCTCTCTCCCAGCGACACATGAGTTACACATCTTTGGCTCCATCAACGGTGTGG
ACTTTGACATGGTGGGTCAGGGCACCGGCAATCCAAATGATGGTTATGAGGAGTTA
AACCTGAAGTCCACCAAGGGTGACCTCCAGTTCTCCCCCTGGATTCTGGTCCCTCAT
ATCGGGTATGGCTTCCATCAGTACCTGCCCTACCCTGACGGGATGTCGCCTTTCCAG
GCCGCCATGGTAGATGGCTCCGGCTACCAAGTCCATCGCACAATGCAGTTTGAAGAT
GGTGCCTCCCTTACTGTTAACTACCGCTACACCTACGAGGGAAGCCACATCAAAGGA
GAGGCCCAGGTGAAGGGGACTGGTTTCCCTGCTGACGGTCCTGTGATGACCAACTCG
CTGACCGCTGCGGACTGGTGCAGGTCGAAGAAGACTTACCCCAACGACAAAACCAT
CATCAGTACCTTTAAGTGGAGTTACACCACTGGAAATGGCAAGCGCTACCGGAGCA
CTGCGCGGACCACCTACACCTTTGCCAAGCCAATGGCGGCTAACTATCTGAAGAACC
AGCCGATGTACGTGTTCCGTAAGACGGAGCTCAAGCACTCCAAGACCGAGCTCAAC
TTCAAGGAGTGGCAAAAGGCCTTTACCGATGTGATGGGCATGGACGAGCTGTACAA
GTCCGGACTCAGATCTCGAGCTCAAGCTTCGATGCTGGAACGAAGGGCGTTGCTATG
GCAACGGGAGGCAGGGCCTGGTTGGGGGGACCGGGCCCGGGCTGGGACCGGGGGG
GCCGGCGGTGGCTGTGGTGGGGCGATGGCGGAGCGCGGCCCGGCCTTCTGCGGCCT
GTACGACACGTCCTCGCTGCTGCGATACTGCAACGATGACAATTTGTCGGGCACGAG
CGGTATGGAAGTGGACGACCGCGTGTCGGCGCTGGAGCAGCGGCTGCAGTTACAGG
AAGACGAGCTGGCGGTCCTAAAGGCGGCGCTGGCGGATGCTCTGCGTCGCCTGCGG
GCATGCGAAGAACAGGGAGCGGCGCTACGCGCGCGGGGCACCCCCAAGGGCCGGG
CGCCTCCGCGCTTAGGCACCACTGCCTCGGTGTGTCAGCTCTTGAAAGGCCTTCCCA
CCAGGACGCCCCTTAATGGCTCGGGACCCCCGCGGCGCGTGGGTGGCTATGCCACG
TCCCCATCCTCTCCCAAGAAGGAGGCGACCTCCGGGCGCAGCAGTGTCCGCCGCTAC
TTGTCACCAGAGCGCCTCGCCTCGGTGCGCCGTGAGGACCCCCGCAGCCGGACCAC
ATCCTCCAGCAGCAACTGTAGCGCCAAAAAGGAAGGCAAAACCAAAGAAGTTATCT
TCAGTGTGGAGGATGGCTCCGTGAAAATGTTCCTGAGGGGCCGCCCTGTGCCCATGA
TGATCCCAGACGAGCTGGCACCCACCTACAGCCTGGACACACGCTCGGAGCTGCCTT
CTTGCCGGCTCAAGCTGGAGTGGGTCTATGGCTACCGTGGCCGAGACTGCCGGGCC
AACCTTTATTTGCTGCCCACCGGGGAGATAGTGTACTTTGTGGCCTCCGTAGCCGTG
CTATACAGCGTGGAGGAGCAGAGGCAGCGACACTACCTGGGACACAACGATGACAT
CAAATGCTTGGCCATCCACCCAGATATGGTCACCATCGCCACGGGACAGGTGGCGG
GAACCACTAAGGAAGGGAAGCCGCTGCCGCCCCACGTGCGCATCTGGGACTCAGTT
TCCCTCTCCACCTTACACGTGCTGGGCTTGGGGGTGTTTGACAGAGCCGTGTGCTGT
GTGGGCTTCTCCAAATCTAATGGAGGCAACCTGCTGTGTGCAGTGGATGAATCCAAT
GATCACATGCTCTCGGTGTGGGACTGGGCCAAGGAGACCAAGGTGGTGGATGTCAA
GTGCTCCAATGAGGCTGTATTGGTGGCCACCTTCCACCCCACGGACCCCACTGTGCT
TATCACCTGCGGGAAATCTCACATCTACTTCTGGACCTTGGAGGGGGGCAGCTTGAG
CAAGCGGCAAGGCCTCTTTGAGAAACATGAGAAACCGAAGTATGTGCTGTGTGTGA
CCTTTTTGGAAGGTGGCGACGTGGTCACGGGGGACTCTGGGGGGAACCTCTATGTTT
GGGGCAAAGGTGGGAACCGTATCACACAGGCGGTGCTGGGCGCCCACGACGGCGG
CGTGTTTGGGCTCTGCGCCCTGCGGGACGGGACGCTGGTGTCTGGAGGGGGCCGTG
ATCGGCGGGTGGTCCTCTGGGGTTCTGACTACAGCAAGCTGCAGGAAGTGGAGGTC
CCTGAGGACTTTGGCCCTGTGCGCACCGTGGCAGAGGGCCACGGAGACACACTGTA
CGTGGGGACCACCCGCAATTCCATCCTGCAGGGCTCCGTGCACACAGGCTTCTCACT
GCTGGTCCAGGGCCATGTGGAAGAGCTGTGGGGCCTGGCCACACACCCCAGTCGGG
CCCAGTTTGTGACCTGCGGGCAGGATAAGCTGGTGCATCTATGGAGCTCAGATTCCC
ACCAGCCCCTGTGGAGCAGGATCATCGAGGACCCTGCCCGCTCAGCCGGCTTCCACC
CCAGTGGCTCTGTCCTGGCTGTGGGTACAGTGACTGGCAGATGGCTGCTGCTGGACA
CGGAGACCCATGACCTGGTGGCTATCCACACAGACGGCAATGAACAGATCTCAGTG
GTCAGCTTCTCCCCAGACGGGGCGTACCTGGCCGTGGGCTCCCACGACAACTTGGTG
TACGTGTACACGGTGGACCAGGGCGGCCGCAAGGTCAGCCGCCTGGGCAAGTGCTC
GGGCCATTCCAGTTTTATCACCCACCTGGATTGGGCCCAGGACAGCAGCTGCTTTGT
CACCAACTCCGGGGACTATGAGATTCTGTACTGGGACCCGGCTACCTGTAAGCAGAT
CACCAGTGCGGATGCTGTGAGGAACATGGAATGGGCCACAGCTACTTGTGTCCTAG
GGTTTGGGGTGTTTGGGATCTGGTCTGAGGGGGCGGACGGCACTGATATCAACGCTG
TGGCCCGCTCTCATGATGGGAAGTTGCTGGCTTCAGCTGATGACTTTGGCAAAGTTC
ACCTGTTTAGCTACCCCTGCTGTCAGCCTCGAGCCCTCAGCCACAAGTACGGTGGAC
ACAGCAGCCATGTGACAAATGTGGCCTTCTTGTGGGATGACAGCATGGCCCTGACCA
CAGGGGGCAAGGACACCAGTGTGCTACAGTGGCGGGTGGTCTGACTGCAGTCGACG
GTACCGCGGGCCCGGGATCCACCGGATCTAGATAACTGATCATAATCAGCCATACC
ACATTTGTAGAGGTTTTACTTGCTTTAAAAAACCTCCCACACCTCCCCCTGAACCTGA
AACATAAAATGAATGCAATTGTTGTTGTTAACTTGTTTATTGCAGCTTATAATGGTTA
CAAATAAAGCAATAGCATCACAAATTTCACAAATAAAGCATTTTTTTCACTGCATTC
TAGTTGTGGTTTGTCCAAACTCATCAATGTATCTTAACGCGTAAATTGTAAGCGTTA
ATATTTTGTTAAAATTCGCGTTAAATTTTTGTTAAATCAGCTCATTTTTTAACCAATA
GGCCGAAATCGGCAAAATCCCTTATAAATCAAAAGAATAGACCGAGATAGGGTTGA
GTGTTGTTCCAGTTTGGAACAAGAGTCCACTATTAAAGAACGTGGACTCCAACGTCA
AAGGGCGAAAAACCGTCTATCAGGGCGATGGCCCACTACGTGAACCATCACCCTAA
TCAAGTTTTTTGGGGTCGAGGTGCCGTAAAGCACTAAATCGGAACCCTAAAGGGAG
CCCCCGATTTAGAGCTTGACGGGGAAAGCCGGCGAACGTGGCGAGAAAGGAAGGG
AAGAAAGCGAAAGGAGCGGGCGCTAGGGCGCTGGCAAGTGTAGCGGTCACGCTGC
GCGTAACCACCACACCCGCCGCGCTTAATGCGCCGCTACAGGGCGCGTCAGGTGGC
ACTTTTCGGGGAAATGTGCGCGGAACCCCTATTTGTTTATTTTTCTAAATACATTCAA
ATATGTATCCGCTCATGAGACAATAACCCTGATAAATGCTTCAATAATATTGAAAAA
GGAAGAGTCCTGAGGCGGAAAGAACCAGCTGTGGAATGTGTGTCAGTTAGGGTGTG
GAAAGTCCCCAGGCTCCCCAGCAGGCAGAAGTATGCAAAGCATGCATCTCAATTAG
TCAGCAACCAGGTGTGGAAAGTCCCCAGGCTCCCCAGCAGGCAGAAGTATGCAAAG
CATGCATCTCAATTAGTCAGCAACCATAGTCCCGCCCCTAACTCCGCCCATCCCGCC
CCTAACTCCGCCCAGTTCCGCCCATTCTCCGCCCCATGGCTGACTAATTTTTTTTATT
TATGCAGAGGCCGAGGCCGCCTCGGCCTCTGAGCTATTCCAGAAGTAGTGAGGAGG
CTTTTTTGGAGGCCTAGGCTTTTGCAAAGATCGATCAAGAGACAGGATGAGGATCGT
TTCGCATGATTGAACAAGATGGATTGCACGCAGGTTCTCCGGCCGCTTGGGTGGAGA
GGCTATTCGGCTATGACTGGGCACAACAGACAATCGGCTGCTCTGATGCCGCCGTGT
TCCGGCTGTCAGCGCAGGGGCGCCCGGTTCTTTTTGTCAAGACCGACCTGTCCGGTG
CCCTGAATGAACTGCAAGACGAGGCAGCGCGGCTATCGTGGCTGGCCACGACGGGC
GTTCCTTGCGCAGCTGTGCTCGACGTTGTCACTGAAGCGGGAAGGGACTGGCTGCTA
TTGGGCGAAGTGCCGGGGCAGGATCTCCTGTCATCTCACCTTGCTCCTGCCGAGAAA
GTATCCATCATGGCTGATGCAATGCGGCGGCTGCATACGCTTGATCCGGCTACCTGC
CCATTCGACCACCAAGCGAAACATCGCATCGAGCGAGCACGTACTCGGATGGAAGC
CGGTCTTGTCGATCAGGATGATCTGGACGAAGAGCATCAGGGGCTCGCGCCAGCCG
AACTGTTCGCCAGGCTCAAGGCGAGCATGCCCGACGGCGAGGATCTCGTCGTGACC
CATGGCGATGCCTGCTTGCCGAATATCATGGTGGAAAATGGCCGCTTTTCTGGATTC
ATCGACTGTGGCCGGCTGGGTGTGGCGGACCGCTATCAGGACATAGCGTTGGCTACC
CGTGATATTGCTGAAGAGCTTGGCGGCGAATGGGCTGACCGCTTCCTCGTGCTTTAC
GGTATCGCCGCTCCCGATTCGCAGCGCATCGCCTTCTATCGCCTTCTTGACGAGTTCT
TCTGAGCGGGACTCTGGGGTTCGAAATGACCGACCAAGCGACGCCCAACCTGCCAT
CACGAGATTTCGATTCCACCGCCGCCTTCTATGAAAGGTTGGGCTTCGGAATCGTTT
TCCGGGACGCCGGCTGGATGATCCTCCAGCGCGGGGATCTCATGCTGGAGTTCTTCG
CCCACCCTAGGGGGAGGCTAACTGAAACACGGAAGGAGACAATACCGGAAGGAAC
CCGCGCTATGACGGCAATAAAAAGACAGAATAAAACGCACGGTGTTGGGTCGTTTG
TTCATAAACGCGGGGTTCGGTCCCAGGGCTGGCACTCTGTCGATACCCCACCGAGAC
CCCATTGGGGCCAATACGCCCGCGTTTCTTCCTTTTCCCCACCCCACCCCCCAAGTTC
GGGTGAAGGCCCAGGGCTCGCAGCCAACGTCGGGGCGGCAGGCCCTGCCATAGCCT
CAGGTTACTCATATATACTTTAGATTGATTTAAAACTTCATTTTTAATTTAAAAGGAT
CTAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGA
GCGCAGATACCAAATACTGTCCTTCTAGTGTAGCCGTAGTTAGGCCACCACTTCAAG
AACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGAT
AAGGCGCAGCGGTCGGGCTGAACGGGGGGTTCGTGCACACAGCCCAGCTTGGAGCG
AACGACCTACACCGAACTGAGATACCTACAGCGTGAGCTATGAGAAAGCGCCACGC
TTCCCGAAGGGAGAAAGGCGGACAGGTATCCGGTAAGCGGCAGGGTCGGAACAGG
AGAGCGCACGAGGGAGCTTCCAGGGGGAAACGCCTGGTATCTTTATAGTCCTGTCG
GGTTTCGCCACCTCTGACTTGAGCGTCGATTTTTGTGATGCTCGTCAGGGGGGCGGA
GCCTATGGAAAAACGCCAGCAACGCGGCCTTTTTACGGTTCCTGGCCTTTTGCTGGC
CTTTTGCTCACATGTTCTTTCCTGCGTTATCCCCTGATTCTGTGGATAACCGTATTACC
GCCATGCAT

We claim:

1. A method for evaluating microtubule length, comprising: contacting a sample comprising microtubules with an EML protein, and detecting a shortening of microtubule length.

2. The method of claim 1, wherein said microtubules are in a cell in vitro.

3. The method of claim 1, wherein said microtubules are in a cell in vivo.

4. The method of claim 1, wherein said EML protein is an EML2 protein or variant or fragment thereof.

5. The method of claim 1, wherein said EML protein is labeled.

6. The method of claim 4, wherein said EML protein is labeled with a fluorescent moiety.

7. The method of claim 1, wherein said microtubules are further contacted with an agent that identifies lengthening of microtubules.

8. The method of claim 7, wherein said agent that identifies lengthening of microtubules comprises a protein selected from the group consisting of EB1, EB3, and CLIP-170.

9. The method of claim 7, wherein said EML protein is labeled with a first label and said agent that identifies lengthening is labeled with a second, different label.

10. A method for analyzing an agent, comprising: exposing a sample comprising microtubules to an agent and an EML protein, and determining an ability of the agent to alter microtubule length.

11. The method of claim 10, wherein said agent is a drug.

12. A kit comprising: a labeled EML protein and a labeled protein that detects microtubule lengthening.

13. The kit of claim 12, wherein said protein that detects microtubule lengthening is selected from the group consisting of EB1, EB3, and CLIP-170.

14. A reaction mixture comprising: a) a cell having microtubules; and b) a labeled EML protein.

15. The reaction mixture of claim 14, further comprising a labeled protein that detects microtubule lengthening.

16. The reaction mixture of claim 15, wherein said protein that detects microtubule lengthening is selected from the group consisting of EB1, EB3, and CLIP-170.

17. A composition comprising an expression vector that encodes EML2 and a protein that detects microtubule lengthening.

18. The composition of claim 17, wherein said protein that detects microtubule lengthening is selected from the group consisting of EB1, EB3, and CLIP-170

19. A composition comprising a cell containing an expression vector that encodes EML2 and a protein that detects microtubule lengthening.

20. The composition of claim 19, wherein said protein that detects microtubule lengthening is selected from the group consisting of EB1, EB3, and CLIP-170


SEQUENCE LISTING (109 sequences)

SEQ ID NO: 1 - protein, 649 aa, Homo sapiens
MSSFGAGKTKEVIFSVEDGSVKMFLRGRPVPMMIPDELAPTYSLDTRSELPSCRLKLEWVYGYRGRDCRANLYLLPTGEIVYFVASVAVLYSVEEQRQRHYLGHNDDIKCLAIHPDMVTIATGQVAGTTKEGKPLPPHVRIWDSVSLSTLHVLGLGVFDRAVCCVGFSKSNGGNLLCAVDESNDHMLSVWDWAKETKVVDVKCSNEAVLVATFHPTDPTVLITCGKSHIYFWTLEGGSLSKRQGLFEKHEKPKYVLCVTFLEGGDVVTGDSGGNLYVWGKGGNRITQAVLGAHDGGVFGLCALRDGTLVSGGGRDRRVVLWGSDYSKLQEVEVPEDFGPVRTVAEGHGDTLYVGTTRNSILQGSVHTGFSLLVQGHVEELWGLATHPSRAQFVTCGQDKLVHLWSSDSHQPLWSRIIEDPARSAGFHPSGSVLAVGTVTGRWLLLDTETHDLVAIHTDGNEQISVVSFSPDGAYLAVGSHDNLVYVYTVDQGGRKVSRLGKCSGHSSFITHLDWAQDSSCFVTNSGDYEILYWDPATCKQITSADAVRNMEWATATCVLGFGVFGIWSEGADGTDINAVARSHDGKLLASADDFGKVHLFSYPCCQPRALSHKYGGHSSHVTNVAFLWDDSMALTTGGKDTSVLQWRVV

SEQ ID NO: 2 - protein, 773 aa, Canis sp.
MSESGGDFLPLGALFLAHLPASQAAADDNLSGTSGMEVDDRVSALEQRLQLQEDELAVLKAALADALRRLRACEEQGAALRARGTPKGRAPPRLGTTASVCQLLKGLPTRTPLNGAGPPRRVGGYATSPSSPKKEASSGRSARRYLSPERLASVRREDPRSRTTSSSSNCSAKKEGKTKEVIFSLEEGSVKMFLRGRPVPMLIPDEMAPTYSLDTRSELPPSRLKLDWVYGYRGRDCRANLYLLPTGEIVYFVASVAVLYSVEEQRQRHYLGHNDDIKCLAVHPDMVTIATGQVAGTTKEGKPLPPHVRIWDSVSLSTLHVLGLGVFDRAVCCVGFSKSNGGNLLCAVDESNDHMLSVWDWAKEAKVVDGKKHEKPKYVLCVTFLEGGDVVTGDSGGNLYVWGKGGNRITQAVLGAHDGGVFGLCALRDGTLVSGGGRDRRVVLWGSDYSKLQEVEVPEDFGPVRTVAEGRGDTLYVGTTRNSILQGSVHTGFSLLVQGHVEELWGLATHPSRAQFVTCGQDKLVHLWSAESHQPLWSRIIEDPARSAGFHPSGSVLAVGTVTGRWLLLDTETHDLVAIHTDGNEQISVVSFSPDGAYLTVGSHDNLVYVYTVDQGGRKVSRLGKCSGHSSFITHLDWAQDSSCFVTNSGDYEILYWDPATCKQITSADAVRNVEWATATCVLGFGVFGIWSEGADGTDINAVARSHDGKLLASADDFGKVHLFSYPCCQPRALSHKYGGHSSHVTNVAFLWDDSMALTTGGKDTSVLQWRVV

SEQ ID NO: 3 - protein, 649 aa, Mus musculus
MSSFGIGKTKEVIFSMEEGSVKMFLRGRPVPMLIPDELAPTYSLDTRSELPSSRLKLDWVYGYRGRDCRANLYLLPTGEVVYFVASVAVLYSVEEQRQRHYLGHNDDIKCLAVHPDMVTIATGQVAGTTKEGKPLPPHVRVWDSVSLSTLHVLGLGVFDRAVCCVAFSKSNGGNLLCAVDESNDHVLSVWDWAKESKVVDSKCSNEAVLVATFHPTDPSLLITCGKSHIYFWSLEGGSLSKRQGLFEKHEKPKYVLCVTFLEGGDVVTGDSGGNLYVWGKGGNRITQEVQGAHDGGVFALCALRDGTLVSGGGRDRRVVLWGSDYSKVQEVEVPEDFGPVRTVAEGRGDTLYVGTTRNSILLGSVHTGFSLLVQGHVEELWGLATHPSRAQFVTCGQDKLVHLWSSETHQPVWSRSIEDPARSAGFHPSGSVLAVGTVTGRWLLLDTETHDLVAIHTDGNEQISVVSFSPDGAYLAVGSHDNLVYVYTVDQGGRKVSRLGKCSGHSSFITHLDWAQDSTCFVTNSGDYEILYWDPVTCKQITSADTVRNVEWATATCVLGFGVFGIWPEGADGTDINAVARSHDGKLLVSADDFGKVHLFSYPCCQPRALSHKYGGHSSHVTNVAFLWDDSMALTTGGKDTSVLQWRVA

SEQ ID NO: 4 - protein, 649 aa, Rattus norvegicus
MSSFGTGKTKEVIFSMEEGSVKMFLRGRPVPMLIPDELAPTYSLDTRSELPSSRLKLDWVYGYRGRDCRANLYLLPTGEVVYFVASVAVLYSVEEQRQRHYLGHNDDIKCLAVHPDMVTIATGQVAGTTKEGKPLPPHVRVWDSVSLSTLHVLGLGVFDRAVCCVAFSKSNGGNLLCAVDESNDHVLSVWDWAKESKVVDSKCSNEAVLVATFHPTDPNLLITCGKSHIYFWSLEGGNLSKRQGLFEKHEKPKYVLCVTFLEGGDVVTGDSGGNLYVWGKGGNRITQEVLGAHDGGVFALCALRDGTLVSGGGRDRRVVLWGSDYSKVQEVEVPEDFGPVRTVAEGRGDTLYVGTTRNSILLGSVHTGFSLLVQGHVEELWGLATHPSRAQFVSCGQDKLVHLWSSETHQPVWSRSIEDPARSAGFHPSGSVLAVGTVTGRWLLLDTDTRDLVAIHTDGNEQISVVSFSPDGAYLAVGSHDNLVYVYTVDQGGRKVSRLGKCSGHSSFITHLDWAQDSTCFVTNSGDYEILYWDAATCKQITSADTVRNVQWATATCVLGFGVFGIWPEGADGTDINAVARSHDGNLLVSADDFGKVHLFSYPCCQPRALSHKYGGHSSHVTNVAFLWDDSMVLTTGGKDTSVLQWRVA

SEQ ID NO: 5 - DNA, 7271 bp, synthetic construct
  source 1..7271: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtccggactcagatctcgagctcaagcttcgatgctggaacgaagggcgttgctatggcaacgggaggcagggcctggttggggggaccgggcccgggctgggaccgggggggccggcggtggctgtggtggggcgatggcggagcgcggcccggccttctgcggcctgtacgacacgtcctcgctgctgcgatactgcaacgatgacaatttgtcgggcacgagcggtatggaagtggacgaccgcgtgtcggcgctggagcagcggctgcagttacaggaagacgagctggcggtcctaaaggcggcgctggcggatgctctgcgtcgcctgcgggcatgcgaagaacagggagcggcgctacgcgcgcggggcacccccaagggccgggcgcctccgcgcttaggcaccactgcctcggtgtgtcagctcttgaaaggccttcccaccaggacgccccttaatggctcgggacccccgcggcgcgtgggtggctatgccacgtccccatcctctcccaagaaggaggcgacctccgggcgcagcagtgtccgccgctacttgtcaccagagcgcctcgcctcggtgcgccgtgaggacccccgcagccggaccacatcctccagcagcaactgtagcgccaaaaaggaaggcaaaaccaaagaagttatcttcagtgtggaggatggctccgtgaaaatgttcctgaggggccgccctgtgcccatgatgatcccagacgagctggcacccacctacagcctggacacacgctcggagctgccttcttgccggctcaagctggagtgggtctatggctaccgtggccgagactgccgggccaacctttatttgctgcccaccggggagatagtgtactttgtggcctccgtagccgtgctatacagcgtggaggagcagaggcagcgacactacctgggacacaacgatgacatcaaatgcttggccatccacccagatatggtcaccatcgccacgggacaggtggcgggaaccactaaggaagggaagccgctgccgccccacgtgcgcatctgggactcagtttccctctccaccttacacgtgctgggcttgggggtgtttgacagagccgtgtgctgtgtgggcttctccaaatctaatggaggcaacctgctgtgtgcagtggatgaatccaatgatcacatgctctcggtgtgggactgggccaaggagaccaaggtggtggatgtcaagtgctccaatgaggctgtattggtggccaccttccaccccacggaccccactgtgcttatcacctgcgggaaatctcacatctacttctggaccttggaggggggcagcttgagcaagcggcaaggcctctttgagaaacatgagaaaccgaagtatgtgctgtgtgtgacctttttggaaggtggcgacgtggtcacgggggactctggggggaacctctatgtttggggcaaaggtgggaaccgtatcacacaggcggtgctgggcgcccacgacggcggcgtgtttgggctctgcgccctgcgggacgggacgctggtgtctggagggggccgtgatcggcgggtggtcctctggggttctgactacagcaagctgcaggaagtggaggtccctgaggactttggccctgtgcgcaccgtggcagagggccacggagacacactgtacgtggggaccacccgcaattccatcctgcagggctccgtgcacacaggcttctcactgctggtccagggccatgtggaagagctgtggggcctggccacacaccccagtcgggcccagtttgtgacctgcgggcaggataagctggtgcatctatggagctcagattcccaccagcccctgtggagcaggatcatcgaggaccctgcccgctcagccggcttccaccccagtggctctgtcctggctgtgggtacagtgactggcagatggctgctgctggacacggagacccatgacctggtggctatccacacagacggcaatgaacagatctcagtggtcagcttctccccagacggggcgtacctggccgtgggctcccacgacaacttggtgtacgtgtacacggtggaccagggcggccgcaaggtcagccgcctgggcaagtgctcgggccattccagttttatcacccacctggattgggcccaggacagcagctgctttgtcaccaactccggggactatgagattctgtactgggacccggctacctgtaagcagatcaccagtgcggatgctgtgaggaacatggaatgggccacagctacttgtgtcctagggtttggggtgtttgggatctggtctgagggggcggacggcactgatatcaacgctgtggcccgctctcatgatgggaagttgctggcttcagctgatgactttggcaaagttcacctgtttagctacccctgctgtcagcctcgagccctcagccacaagtacggtggacacagcagccatgtgacaaatgtggccttcttgtgggatgacagcatggccctgaccacagggggcaaggacaccagtgtgctacagtggcgggtggtctgactgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 6 - DNA, 7271 bp, synthetic construct
  source 1..7271: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtccggactcagatctcgagctcaagcttcgatgctggaacgaagggcgttgctatggcaacgggaggcagggcctggttggggggaccgggcccgggctgggaccgggggggccggcggtggctgtggtggggcgatggcggagcgcggcccggccttctgcggcctgtacgacacgtcctcgctgctgcgatactgcaacgatgacaatttgtcgggcacgagcggtatggaagtggacgaccgcgtgtcggcgctggagcagcggctgcagttacaggaagacgagctggcggtcctaaaggcggcggcggcggctgctctgcgtcgcctgcgggcatgcgaagaacagggagcggcgctacgcgcgcggggcacccccaagggccgggcgcctccgcgcttaggcaccactgcctcggtgtgtcagctcttgaaaggccttcccaccaggacgccccttaatggctcgggacccccgcggcgcgtgggtggctatgccacgtccccatcctctcccaagaaggaggcgacctccgggcgcagcagtgtccgccgctacttgtcaccagagcgcctcgcctcggtgcgccgtgaggacccccgcagccggaccacatcctccagcagcaactgtagcgccaaaaaggaaggcaaaaccaaagaagttatcttcagtgtggaggatggctccgtgaaaatgttcctgaggggccgccctgtgcccatgatgatcccagacgagctggcacccacctacagcctggacacacgctcggagctgccttcttgccggctcaagctggagtgggtctatggctaccgtggccgagactgccgggccaacctttatttgctgcccaccggggagatagtgtactttgtggcctccgtagccgtgctatacagcgtggaggagcagaggcagcgacactacctgggacacaacgatgacatcaaatgcttggccatccacccagatatggtcaccatcgccacgggacaggtggcgggaaccactaaggaagggaagccgctgccgccccacgtgcgcatctgggactcagtttccctctccaccttacacgtgctgggcttgggggtgtttgacagagccgtgtgctgtgtgggcttctccaaatctaatggaggcaacctgctgtgtgcagtggatgaatccaatgatcacatgctctcggtgtgggactgggccaaggagaccaaggtggtggatgtcaagtgctccaatgaggctgtattggtggccaccttccaccccacggaccccactgtgcttatcacctgcgggaaatctcacatctacttctggaccttggaggggggcagcttgagcaagcggcaaggcctctttgagaaacatgagaaaccgaagtatgtgctgtgtgtgacctttttggaaggtggcgacgtggtcacgggggactctggggggaacctctatgtttggggcaaaggtgggaaccgtatcacacaggcggtgctgggcgcccacgacggcggcgtgtttgggctctgcgccctgcgggacgggacgctggtgtctggagggggccgtgatcggcgggtggtcctctggggttctgactacagcaagctgcaggaagtggaggtccctgaggactttggccctgtgcgcaccgtggcagagggccacggagacacactgtacgtggggaccacccgcaattccatcctgcagggctccgtgcacacaggcttctcactgctggtccagggccatgtggaagagctgtggggcctggccacacaccccagtcgggcccagtttgtgacctgcgggcaggataagctggtgcatctatggagctcagattcccaccagcccctgtggagcaggatcatcgaggaccctgcccgctcagccggcttccaccccagtggctctgtcctggctgtgggtacagtgactggcagatggctgctgctggacacggagacccatgacctggtggctatccacacagacggcaatgaacagatctcagtggtcagcttctccccagacggggcgtacctggccgtgggctcccacgacaacttggtgtacgtgtacacggtggaccagggcggccgcaaggtcagccgcctgggcaagtgctcgggccattccagttttatcacccacctggattgggcccaggacagcagctgctttgtcaccaactccggggactatgagattctgtactgggacccggctacctgtaagcagatcaccagtgcggatgctgtgaggaacatggaatgggccacagctacttgtgtcctagggtttggggtgtttgggatctggtctgagggggcggacggcactgatatcaacgctgtggcccgctctcatgatgggaagttgctggcttcagctgatgactttggcaaagttcacctgtttagctacccctgctgtcagcctcgagccctcagccacaagtacggtggacacagcagccatgtgacaaatgtggccttcttgtgggatgacagcatggccctgaccacagggggcaaggacaccagtgtgctacagtggcgggtggtctgactgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 7 - DNA, 9401 bp, synthetic construct
  source 1..9401: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggccgtcaatgtgtactccacatctgtgaccagtgaaaatctgagtcgccatgatatgcttgcatgggtcaacgactccctgcacctcaactataccaagatagaacagctttgttcaggggcagcctactgccagttcatggacatgctcttccccggctgtgtgcacttgaggaaagtgaagttccaggccaaactagagcatgaatacatccacaacttcaaggtgctgcaagcagctttcaagaagatgggtgttgacaaaatcattcctgtagagaaattagtgaaaggaaaattccaagataattttgagtttattcagtggtttaagaaattctttgacgcaaactatgatggaaaggattacaaccctctgctggcgcggcagggccaggacgtagcgccacctcctaacccaggtgatcagatcttcaacaaatccaagaaactcattggcacagcagttccacagaggacgtcccccacaggcccaaaaaacatgcagacctctggccggctgagcaatgtggcccccccctgcattctccggaagaatcctccatcagcccgaaatggcggccatgagactgatgcccaaattcttgaactcaaccaacagctggtggacttgaagctgacagtggatgggctggagaaggaacgtgacttctacttcagcaaacttcgtgacatcgagctcatctgccaggagcatgaaagtgaaaacagccctgttatctcaggcatcattggcatcctctatgccacagaggaaggattcgcaccccctgaggacgatgagattgaagagcatcaacaagaagaccaggacgagtacctggatcccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaacccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtccggactcagatctcgagctcaagcttcgatgctggaacgaagggcgttgctatggcaacgggaggcagggcctggttggggggaccgggcccgggctgggaccgggggggccggcggtggctgtggtggggcgatggcggagcgcggcccggccttctgcggcctgtacgacacgtcctcgctgctgcgatactgcaacgatgacaatttgtcgggcacgagcggtatggaagtggacgaccgcgtgtcggcgctggagcagcggctgcagttacaggaagacgagctggcggtcctaaaggcggcgctggcggatgctctgcgtcgcctgcgggcatgcgaagaacagggagcggcgctacgcgcgcggggcacccccaagggccgggcgcctccgcgcttaggcaccactgcctcggtgtgtcagctcttgaaaggccttcccaccaggacgccccttaatggctcgggacccccgcggcgcgtgggtggctatgccacgtccccatcctctcccaagaaggaggcgacctccgggcgcagcagtgtccgccgctacttgtcaccagagcgcctcgcctcggtgcgccgtgaggacccccgcagccggaccacatcctccagcagcaactgtagcgccaaaaaggaaggcaaaaccaaagaagttatcttcagtgtggaggatggctccgtgaaaatgttcctgaggggccgccctgtgcccatgatgatcccagacgagctggcacccacctacagcctggacacacgctcggagctgccttcttgccggctcaagctggagtgggtctatggctaccgtggccgagactgccgggccaacctttatttgctgcccaccggggagatagtgtactttgtggcctccgtagccgtgctatacagcgtggaggagcagaggcagcgacactacctgggacacaacgatgacatcaaatgcttggccatccacccagatatggtcaccatcgccacgggacaggtggcgggaaccactaaggaagggaagccgctgccgccccacgtgcgcatctgggactcagtttccctctccaccttacacgtgctgggcttgggggtgtttgacagagccgtgtgctgtgtgggcttctccaaatctaatggaggcaacctgctgtgtgcagtggatgaatccaatgatcacatgctctcggtgtgggactgggccaaggagaccaaggtggtggatgtcaagtgctccaatgaggctgtattggtggccaccttccaccccacggaccccactgtgcttatcacctgcgggaaatctcacatctacttctggaccttggaggggggcagcttgagcaagcggcaaggcctctttgagaaacatgagaaaccgaagtatgtgctgtgtgtgacctttttggaaggtggcgacgtggtcacgggggactctggggggaacctctatgtttggggcaaaggtgggaaccgtatcacacaggcggtgctgggcgcccacgacggcggcgtgtttgggctctgcgccctgcgggacgggacgctggtgtctggagggggccgtgatcggcgggtggtcctctggggttctgactacagcaagctgcaggaagtggaggtccctgaggactttggccctgtgcgcaccgtggcagagggccacggagacacactgtacgtggggaccacccgcaattccatcctgcagggctccgtgcacacaggcttctcactgctggtccagggccatgtggaagagctgtggggcctggccacacaccccagtcgggcccagtttgtgacctgcgggcaggataagctggtgcatctatggagctcagattcccaccagcccctgtggagcaggatcatcgaggaccctgcccgctcagccggcttccaccccagtggctctgtcctggctgtgggtacagtgactggcagatggctgctgctggacacggagacccatgacctggtggctatccacacagacggcaatgaacagatctcagtggtcagcttctccccagacggggcgtacctggccgtgggctcccacgacaacttggtgtacgtgtacacggtggaccagggcggccgcaaggtcagccgcctgggcaagtgctcgggccattccagttttatcacccacctggattgggcccaggacagcagctgctttgtcaccaactccggggactatgagattctgtactgggacccggctacctgtaagcagatcaccagtgcggatgctgtgaggaacatggaatgggccacagctacttgtgtcctagggtttggggtgtttgggatctggtctgagggggcggacggcactgatatcaacgctgtggcccgctctcatgatgggaagttgctggcttcagctgatgactttggcaaagttcacctgtttagctacccctgctgtcagcctcgagccctcagccacaagtacggtggacacagcagccatgtgacaaatgtggccttcttgtgggatgacagcatggccctgaccacagggggcaaggacaccagtgtgctacagtggcgggtggtctgactgcagtcgacggtaccgcgggcccgggatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 8 - protein, 34 aa, synthetic construct
ENLYFQGGGSPGISGGGGGILDSMGRLELKLNSS

SEQ ID NO: 9 - protein, 46 aa, synthetic construct
ENLYFQGGGSPGISGGGGGILDSMGRLELKLNSSVEGEGEEEGEEY

SEQ ID NO: 10 - protein, 46 aa, synthetic construct
ENLYFQGGGSPGISGGGGGILDSMGRLELKLNSSSVEGEGEEEGEE

SEQ ID NO: 11 - protein, 12 aa, synthetic construct
LQLQEDELAVLK

SEQ ID NO: 12 - protein, 10 aa, synthetic construct
ACEEQGAALR

SEQ ID NO: 13 - protein, 12 aa, synthetic construct
LGTTASVCQLLK

SEQ ID NO: 14 - protein, 15 aa, synthetic construct
GLPTRTPLNGSGPPR

SEQ ID NO: 15 - protein, 13 aa, synthetic construct
RVGGYATSPSSPK

SEQ ID NO: 16 - protein, 12 aa, synthetic construct
VGGYATSPSSPK

SEQ ID NO: 17 - protein, 13 aa, synthetic construct
SRTTSSSSNCSAK

SEQ ID NO: 18 - protein, 11 aa, synthetic construct
TTSSSSNCSAK

SEQ ID NO: 19 - protein, 12 aa, synthetic construct
TTSSSSNCSAKK

SEQ ID NO: 20 - protein, 15 aa, synthetic construct
TTSSSSNCSAKKEGK

SEQ ID NO: 21 - protein, 14 aa, synthetic construct
TKEVIFSVEDGSVK

SEQ ID NO: 22 - protein, 12 aa, synthetic construct
EVIFSVEDGSVK

SEQ ID NO: 23 - protein, 21 aa, synthetic construct
GRPVPMMIPDELAPTYSLDTR

SEQ ID NO: 24 - protein, 7 aa, synthetic construct
SELPSCR

SEQ ID NO: 25 - protein, 10 aa, synthetic construct
HYLGHNDDIK

SEQ ID NO: 26 - protein, 20 aa, synthetic construct
IWDSVSLSTLHVLGLGVFDR

SEQ ID NO: 27 - protein, 9 aa, synthetic construct
AVCCVGFSK

SEQ ID NO: 28 - protein, 25 aa, synthetic construct
SNGGNLLCAVDESNDHMLSVWDWAK

SEQ ID NO: 29 - protein, 15 aa, synthetic construct
SHIYFWTLEGGSLSK

SEQ ID NO: 30 - protein, 16 aa, synthetic construct
SHIYFWTLEGGSLSKR

SEQ ID NO: 31 - protein, 12 aa, synthetic construct
RQGLFEKHEKPK

SEQ ID NO: 32 - protein, 34 aa, synthetic construct
GGNRITQAVLGAHQGGVFGLCALRQGILVSGGGR

SEQ ID NO: 33 - protein, 34 aa, synthetic construct
GGNRITQAVLGAHDGGVFGLCALRDGTLVSGGGR

SEQ ID NO: 34 - protein, 14 aa, synthetic construct
LQEVEVPEDFGPVR

SEQ ID NO: 35 - protein, 16 aa, synthetic construct
TVAEGHGDTLYVGTTR

SEQ ID NO: 36 - protein, 10 aa, synthetic construct
AQFVTCGQDK

SEQ ID NO: 37 - protein, 19 aa, synthetic construct
SAGFHPSGSVLAVGTVTGR

SEQ ID NO: 38 - protein, 9 aa, synthetic construct
QITSADAVR

SEQ ID NO: 39 - protein, 15 aa, synthetic construct
SHDGKLLASADDFGK

SEQ ID NO: 40 - protein, 10 aa, synthetic construct
LLASADDFGK

SEQ ID NO: 41 - protein, 22 aa, synthetic construct
LLASADDFGKVHLFSYPCCQPR

SEQ ID NO: 42 - protein, 12 aa, synthetic construct
VHLFSYPCCQPR

SEQ ID NO: 43 - protein, 21 aa, synthetic construct
WVYGYRGRDCRANLYLLPTGE

SEQ ID NO: 44 - protein, 21 aa, synthetic construct
WVYGYRGRDCRANLYLLPTGE

SEQ ID NO: 45 - protein, 21 aa, synthetic construct
WVYGYRGRDCRNNLYLLPTGE

SEQ ID NO: 46 - protein, 21 aa, synthetic construct
WVYGYRGRDSRSNLPVLRSGE

SEQ ID NO: 47 - protein, 21 aa, synthetic construct
WAYGYRGKDCRANVYLLPTGK

SEQ ID NO: 48 - protein, 48 aa, synthetic construct
RDGTLVSGGGRDRRVVLNGSDYSKLQEVEVPEDFGPVRTVAEGHGDTL

SEQ ID NO: 49 - protein, 48 aa, synthetic construct
RDGTLVSGGGRDRRVVLWGSDYSKLQEVEVPEDFGPVRTVAEGHGDTL

SEQ ID NO: 50 - protein, 48 aa, synthetic construct
RDGTLVSGGGKDRKLISWSGNYQKLRKTEIPEQFGPIRTVAEGKGDVI

SEQ ID NO: 51 - protein, 48 aa, synthetic construct
RDGTVLSGGGRDRRLVQWGPGLVALQEAEIPEHFGAVRAIAEGLGSEL

SEQ ID NO: 52 - protein, 48 aa, synthetic construct
RNGMLLTGGGKDRKIILWDHDLNPEREIEVPDQYGTIRAVAEGKADQF

SEQ ID NO: 53 - protein, 66 aa, synthetic construct
VDVKCSNEAVLVATFHPTDPTVLITCGKSHIYFWTLEGGSLSKRQGLFEKHEKPKYVLCVTFLEGG

SEQ ID NO: 54 - protein, 66 aa, synthetic construct; identical to SEQ ID NO: 53

SEQ ID NO: 55 - protein, 66 aa, synthetic construct
ADVKCSNEAVFAADFHPTDTNIIVTCGKSHLYFWTLEGSSLNKKQGLFEKQEKPKFVLCVTFSENG

SEQ ID NO: 56 - protein, 73 aa, synthetic construct
AEIKSTNDSVLAVGFNPRDSSCIVTSGKSHVHFWNWSGGVGVPGNGTLTRKQGVFGKYKKPKFIPCFVFLPDG

SEQ ID NO: 57 - protein, 66 aa, synthetic construct
AEIKTTNEVVLAVEFHPTDANTIITCGKSHIFFWTWSGNSLTRKQGIFGKYEKPKFVQCLAFLGNG

SEQ ID NO: 58 - protein, 46 aa, synthetic construct
MEVDDRVSALEQRLQLQEDELAVLKAALADALRRLRACEEQGAALR

SEQ ID NO: 59 - protein, 51 aa, synthetic construct
AASSMEVTDRIALSLEQRVQMQEDDIQLLKSALADVVRRLNITEEQQAVLN

SEQ ID NO: 60 - protein, 50 aa, synthetic construct
GTSGMEVDDRVSALEQRLQLQEDELAVLKAALADALRRLRACEEQGAALR

SEQ ID NO: 61 - protein, 49 aa, synthetic construct
GPGDGPAREALQSLSQRLRVQEQEMELVKAALAEALRLLRLQVPPSSLQ

SEQ ID NO: 62 - protein, 50 aa, synthetic construct
AASTSDVQDRLSALESRVQQQEDEITVLKAALADVLRRLAISEDHVASVK

SEQ ID NO: 63 - DNA, 27 bp, synthetic construct
  source 1..27: mol_type = other DNA
agcacccagtccaagctgagcaaagac

SEQ ID NO: 64 - DNA, 36 bp, synthetic construct
  source 1..36: mol_type = other DNA
accctctccttcaaccgatgaattaagcttgagctc

SEQ ID NO: 65 - DNA, 42 bp, synthetic construct
  source 1..42: mol_type = other DNA
gaggaagaaggagaggaatactgactgactgacgatctgcct

SEQ ID NO: 66 - DNA, 39 bp, synthetic construct
  source 1..39: mol_type = other DNA
gaggaagaaggagaggaatgactgactgacgatctgcct

SEQ ID NO: 67 - DNA, 46 bp, synthetic construct
  source 1..46: mol_type = other DNA
gatctcgagctcaagcttcgatgctggaacgaagggcgttgctatg

SEQ ID NO: 68 - DNA, 46 bp, synthetic construct; identical to SEQ ID NO: 67
  source 1..46: mol_type = other DNA

SEQ ID NO: 69 - DNA, 45 bp, synthetic construct
  source 1..45: mol_type = other DNA
cgcggtaccgtcgactgcagaattcttgcggccgccctggtccac

SEQ ID NO: 70 - DNA, 27 bp, synthetic construct
  source 1..27: mol_type = other DNA
gaggatggctccgtgaaaatgttcctg

SEQ ID NO: 71 - DNA, 37 bp, synthetic construct
  source 1..37: mol_type = other DNA
ggtggaccagggcggccgcaaggtcagccgcctgggc

SEQ ID NO: 72 - DNA, 38 bp, synthetic construct
  source 1..38: mol_type = other DNA
cgcggtaccgtcgactgcagtcagaccacccgccactg

SEQ ID NO: 73 - DNA, 59 bp, synthetic construct
  source 1..59: mol_type = other DNA
gatctcgagctcaagcttcgatgagtagctttggagctggcaaaaccaaagaagttatc

SEQ ID NO: 74 - DNA, 38 bp, synthetic construct
  source 1..38: mol_type = other DNA
taccgcgggcccgggatccaatgggcgttgccatgcca

SEQ ID NO: 75 - DNA, 38 bp, synthetic construct
  source 1..38: mol_type = other DNA
ctgcacctgagtgtttactttcagaccacccgccactg

SEQ ID NO: 76 - DNA, 38 bp, synthetic construct
  source 1..38: mol_type = other DNA
gtgccgaagatgatgtggtgctggaacgaagggcgttg

SEQ ID NO: 77 - DNA, 38 bp, synthetic construct
  source 1..38: mol_type = other DNA
ccgcctgaaccgcctccaccgaccacccgccactgtag

SEQ ID NO: 78 - DNA, 38 bp, synthetic construct
  source 1..38: mol_type = other DNA
gtgccgaagatgatgtggtgagtagctttggagctggc

SEQ ID NO: 79 - DNA, 38 bp, synthetic construct
  source 1..38: mol_type = other DNA
acgtaggcctttgaattccgtcagaccacccgccactg

SEQ ID NO: 80 - DNA, 43 bp, synthetic construct
  source 1..43: mol_type = other DNA
tgtattttcagggcgccatgagtagctttggagctggcaaaac

SEQ ID NO: 81 - DNA, 38 bp, synthetic construct
  source 1..38: mol_type = other DNA
tgggctgggtcctgcaggcgtgggcgttgccatgccag

SEQ ID NO: 82 - DNA, 41 bp, synthetic construct
  source 1..41: mol_type = other DNA
taacctcgagtttaaacgcgttagaccacccgccactgtag

SEQ ID NO: 83 - DNA, 23 bp, synthetic construct
  source 1..23: mol_type = other DNA
cagcaccacatcatcttcggcac

SEQ ID NO: 84 - DNA, 23 bp, synthetic construct
  source 1..23: mol_type = other DNA
gaacgaagggcgttgctatggca

SEQ ID NO: 85 - DNA, 23 bp, synthetic construct
  source 1..23: mol_type = other DNA
caccacatcatcttcggcacctg

SEQ ID NO: 86 - DNA, 24 bp, synthetic construct
  source 1..24: mol_type = other DNA
agtagctttggagctggcaaaacc

SEQ ID NO: 87 - DNA, 21 bp, synthetic construct
  source 1..21: mol_type = other DNA
caccacatcatcttcggcacc

SEQ ID NO: 88 - DNA, 22 bp, synthetic construct
  source 1..22: mol_type = other DNA
tccggactcagatctcgagctc

SEQ ID NO: 89 - DNA, 42 bp, synthetic construct
  source 1..42: mol_type = other DNA
ggtgccgaagatgatgtggtggaggacggcttctccagctac

SEQ ID NO: 90 - DNA, 42 bp, synthetic construct
  source 1..42: mol_type = other DNA
ggtgccgaagatgatgtggtggacggggccgcggggcccggt

SEQ ID NO: 91 - DNA, 53 bp, synthetic construct
  source 1..53: mol_type = other DNA
agctcgagatctgagtccggatcaaacgtcgagggaggaggcgggggacaggg

SEQ ID NO: 92 - DNA, 42 bp, synthetic construct
  source 1..42: mol_type = other DNA
ggtgccgaagatgatgtggtggacggtttcgccggcagtctc

SEQ ID NO: 93 - DNA, 42 bp, synthetic construct
  source 1..42: mol_type = other DNA
agctcgagatctgagtccggattaggacgagggcgaagggtc

SEQ ID NO: 94 - DNA, 43 bp, synthetic construct
  source 1..43: mol_type = other DNA
agctcgagatctgagtccggactaaatgacgcgccactgcatg

SEQ ID NO: 95 - DNA, 39 bp, synthetic construct
  source 1..39: mol_type = other DNA
ctgcacctgagtgtttacttctaaatgacgcgccactgc

SEQ ID NO: 96 - DNA, 40 bp, synthetic construct
  source 1..40: mol_type = other DNA
ctgcacctgagtgtttactttcaaacgtcgagggaggagg

SEQ ID NO: 97 - DNA, 38 bp, synthetic construct
  source 1..38: mol_type = other DNA
ctgcacctgagtgtttacttttaggacgagggcgaagg

SEQ ID NO: 98 - DNA, 28 bp, synthetic construct
  source 1..28: mol_type = other DNA
caaataaaggttggccgcgcagtctcgg

SEQ ID NO: 99 - DNA, 26 bp, synthetic construct
  source 1..26: mol_type = other DNA
tttggccctgtggccaccgtggcaga

SEQ ID NO: 100 - DNA, 32 bp, synthetic construct
  source 1..32: mol_type = other DNA
tggagggggcgctgatgcgcgggtggtcctct

SEQ ID NO: 101 - DNA, 28 bp, synthetic construct
  source 1..28: mol_type = other DNA
caaataaaggttggcctcgcagtctcgg

SEQ ID NO: 102 - DNA, 21 bp, synthetic construct
  source 1..21: mol_type = other DNA
ggtggccacccttacagcctc

SEQ ID NO: 103 - DNA, 25 bp, synthetic construct
  source 1..25: mol_type = other DNA
gaaaccgaaggatgtgctgtgtgtg

SEQ ID NO: 104 - DNA, 38 bp, synthetic construct
  source 1..38: mol_type = other DNA
gatctcgagctcaagcttcgatggaggacggcttctcc

SEQ ID NO: 105 - DNA, 38 bp, synthetic construct
  source 1..38: mol_type = other DNA
cgcggtaccgtcgactgcagctaaatgacgcgccactg

SEQ ID NO: 106 - DNA, 38 bp, synthetic construct
  source 1..38: mol_type = other DNA
gatctcgagctcaagcttcgatggacggggccgcgggg

SEQ ID NO: 107 - DNA, 43 bp, synthetic construct
  source 1..43: mol_type = other DNA
cgcggtaccgtcgactgcagtcaaacgtcgagggaggaggcgg

SEQ ID NO: 108 - DNA, 38 bp, synthetic construct
  source 1..38: mol_type = other DNA
gatctcgagctcaagcttcgatggacggtttcgccggc

SEQ ID NO: 109 - DNA, 39 bp, synthetic construct
  source 1..39: mol_type = other DNA
cgcggtaccgtcgactgcagttaggacgagggcgaaggg